(19) United States
(12) Patent Application Publication
(10) Pub. No.: US 2020/0308165 A1
(43) Pub. Date: Oct. 1, 2020

(54) Compounds as Ras Inhibitors and Use Thereof

(71) Applicant: D.E. Shaw Research, LLC, New York, NY (US)

(72) Inventors: Yibing SHAN, Millburn, NJ (US); Venkat MYSORE, South Orange, NJ (US); Fabrizio GIORDANETTO, New York, NY (US); Qi WANG, Waldwick, NJ (US)

(21) Appl. No.: 16/645,379

(22) PCT Filed: Sep. 12, 2018
(86) PCT No.: PCT/US2018/050717

§ 371 Date: Mar. 6, 2020

Related U.S. Application Data
(60) Provisional application No. 62/557,914, filed on Sep. 13, 2017.

(51) Int. Cl. C07D 417/14; C07D 401/14; C07D 401/04; C07D 403/04; C07D 413/14; C07D 403/14

(52) U.S. Cl. CPC C07D 417/14; C07D 401/14; C07D 403/14; C07D 403/04; C07D 413/14; C07D 401/04

(57) ABSTRACT

A compound of Formula (Ia) or (Ib), or a pharmaceutically acceptable salt thereof is described, wherein the substituents are as defined herein. Pharmaceutical compositions comprising the same and method of using the same are also described.

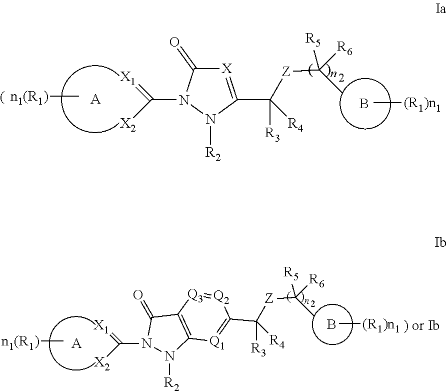

This application claims the benefit of and priority to U.S. Provisional Patent Application No. 62/557,914, filed Sep. 13, 2017, the entire contents of which are hereby incorporated by reference in its entirety.

This patent disclosure contains material that is subject to copyright protection. The copyright owner has no objection to the facsimile reproduction obey anyone of the patent document or the patent disclosure as it appears in the U.S. Patent and Trademark Office patent file or records, but otherwise reserves any and all copyright rights.

### INCORPORATION BY REFERENCE

All documents cited herein are incorporated herein by reference in their entirety.

### FIELD OF THE INVENTION

The invention relates generally to the field of pharmaceutical science. More particularly, the invention relates to compositions useful as pharmaceuticals as anticancer agents.

### BACKGROUND

The Ras proteins belong to a large super-family of proteins known as “low-molecular weight G-proteins.” The Ras proteins control signaling pathways that are key regulators of several aspects of normal cell growth and malignant transformation. In most human tumors, the Ras proteins are abnormal due to activating mutations in the Ras genes themselves or to alterations in upstream or downstream signaling components. Inhibitors of Ras proteins have been developed which might inhibit tumor growth and spread. These inhibitors, however, are either very weak in potency (tens of micromolars in IC₅₀) with unviable properties including large molecular weight and high insolubility, or only applicable to a particular G12C Ras mutant by way forming covalent bond with the cysteine residue unique to the mutant. Some other small molecule inhibitors exhibit moderately improved drug-like properties, but are exceedingly weak in IC₅₀ with potency of hundreds of micromolars. Other than directly targeting Ras proteins, strategies are pursued to target upstream regulators of Ras proteins, such as farnesyl transferase that controls Ras lipidation and membrane localization, or downstream effectors such as B-Raf and PI3K kinases. The therapeutic effect of the inhibitors targeting these Ras-related proteins are typically complicated by compensating pathways in tumor cells.

Thus, there remains a need for novel Ras inhibitors as anticancer agents.

### SUMMARY OF THE INVENTION

In one aspect, compounds useful as Ras protein inhibitors and/or anticancer agents having a structure of Formula Ia or Ib,

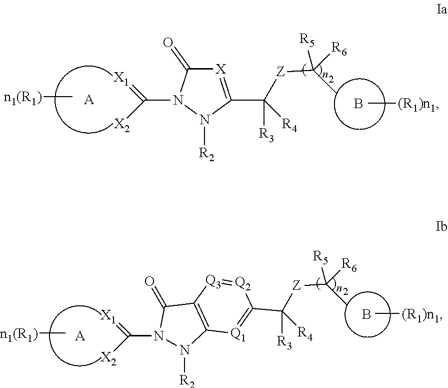

are described, where the various substituents are defined herein. The compounds of Formula Ia and Formula Ib described herein can inhibit Ras protein and thereby act as anticancer agents. Methods for synthesizing these compounds are also described herein. Pharmaceutical compositions and methods of using these compositions described herein are useful for treating cancer in vitro and in vivo. Such compounds, pharmaceutical compositions and methods of treatment have a number of clinical applications, including as pharmaceutically active agents and methods for treating cancers.

In one aspect, a compound of Formula Ia or Ib or a pharmaceutically acceptable salt thereof is described,

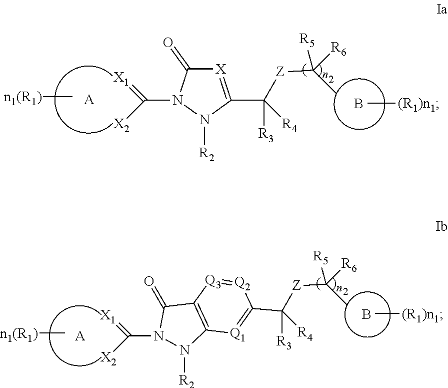

wherein

A is a monocyclic, bicyclic, or tricyclic heterocyclic group optionally substituted by one or more R₁;

each occurrence of n₁ is independently 0, 1, 2, 3, 4, 5, 6, 7, or 8;

X₁ and X₂ are each independently CR₁, O, S, N or NR₂ where valence permits; wherein at least one of X₁ and X₂ is O, N or NR₂;

each occurrence of R₁ is independently hydrogen, halogen, cyano, nitro, —N₃⁻, CF₃, OCF₃, ORₐ, optionally substituted alkyl, optionally substituted cycloalkyl, optionally substituted alkenyl, optionally substituted alkynyl, optionally substituted aryl, optionally substituted heterocycle, —CR_{b}R_{c}-(optionally substituted aryl), —CR_{b}R_{c}-(optionally substituted heteroaryl), —CR_{b}R_{c}—N₃⁻—, SRₐ, S(═O)Rₐ, S(═O)₂Rₐ, —(CR_{b}R_{c})₁₋₄—NR_{b}R_{c}, NR_{b}R_{c}, S(═O)₂NR_{b}R_{c}, oxo, C(═O)ORₐ, C(═O)Rₐ, C(═O)NR_{b}R_{c}, OC(═O)Rₐ, OC(═O)NR_{b}R_{c}, NR_{b}C(═O)ORₐ, or NR_{b}C(═O)Rₐ; or alternatively two R₁ groups substituted on the same ring taken together form an additional 3-7 membered carbocycle or heterocycle optionally substituted by one or more R_{1′};

each occurrence of R_{1′} is independently hydrogen, halogen, cyano, nitro, CF₃, OCF₃, ORₐ, optionally substituted alkyl, optionally substituted cycloalkyl, optionally substituted alkenyl, optionally substituted aryl, optionally substituted heterocycle, SRₐ, oxo, S(═O)Rₐ, S(═O)₂Rₐ, NR_{b}R_{c}, S(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₐ, or NR_{b}C(═O)Rₐ;

each occurrence of R₂ is independently hydrogen, optionally substituted alkyl, optionally substituted cycloalkyl, optionally substituted alkenyl, optionally substituted cycloalkenyl, optionally substituted alkynyl, optionally substituted heterocycle, or optionally substituted aryl;

X is CR₁ or N;

Q₁, Q₂ and Q₃ are each independently CR₁ or N;

each occurrence of R₃ and R₄ is independently hydrogen, alkyl, cycloalkyl, alkenyl, cycloalkenyl, or alkynyl; or alternatively R₃ and R₄ together with the carbon atom that they are connected to form a 3-7 membered optionally substituted carbocycle or heterocycle;

Z is CR₃R₄, NR₂, O, or S;

n₂ is 0 or 1;

each occurrence of R₅ and R₆ is independently hydrogen, alkyl, cycloalkyl, alkenyl, cycloalkenyl, or alkynyl; or alternatively R₅ and R₆ together with the carbon atom that they are connected to form a 3-7 membered optionally substituted carbocycle or heterocycle;

B is absent, or cycloalkyl group or saturated heterocyclic group optionally substituted by one or more R₁, or monocyclic, bicyclic, or tricyclic aryl or heteroaryl group optionally substituted by one or more R₁; and

Rₐ, R_{b}, and R_{c} are each independently hydrogen, alkyl, cycloalkyl, alkenyl, cycloalkenyl, alkynyl, optionally substituted heterocycle, or optionally substituted aryl; or alternatively R_{b}, and R_{c} together with the nitrogen atom that they are connected to form an optionally substituted heterocycle comprising the nitrogen atom and 0-3 additional heteroatoms each selected from the group consisting of N, O, and S.

In some embodiments described herein, the compound has the structure of Formula Ia,

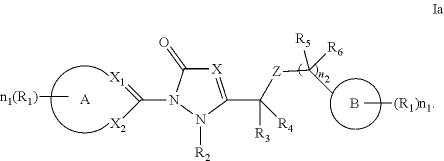

In some embodiments described herein, the compound has the structure of Formula Ib,

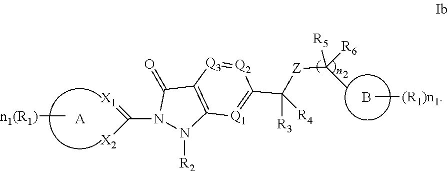

In some embodiments described herein, X₁ is CR₁ and X₂ is N or NR₂ wherein valence permits.

In some embodiments described herein, X₁ is N and X₂ is CR₁, NR₂, O or S.

In some embodiments described herein, X₁ and X₂ are each independently N or NR₂ where valence permits.

In some embodiments described herein, X₁ is N and X₂ is NR₂.

In some embodiments described herein, A is an optionally substituted heterocycle selected from the group consisting of pyrrolyl, pyrazolyl, imidazolyl, isoxazolyl, thiazolyl, thiadiazolyl, isothiazolyl, pyridinyl, pyrazinyl, pyrimidinyl, pyridazinyl, benzothiazolyl and benzoimidazolyl.

In some embodiments described herein, the substituent

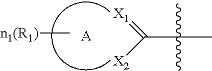

has the structure of

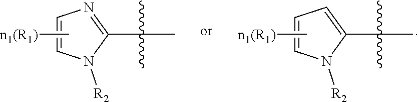

In some embodiments described herein, the substituent

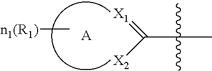

has the structure of

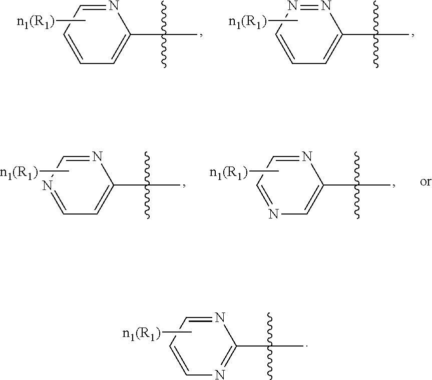

In some embodiments described herein, the substituent

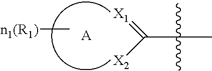

has the structure of

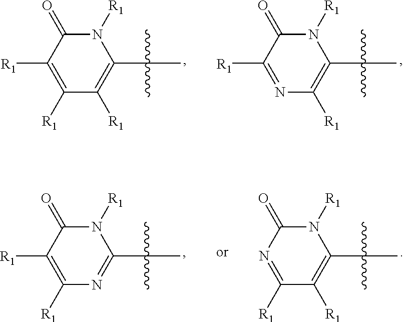

In some embodiments described herein, the substituent

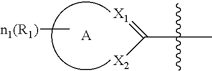

has the structure of

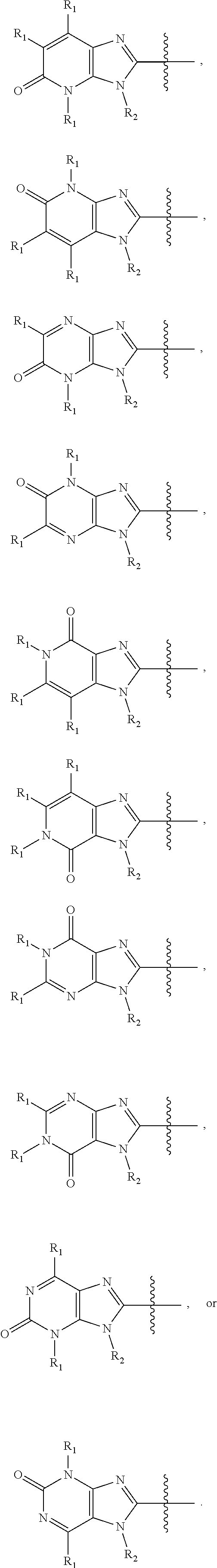

In some embodiments described herein, the substituent

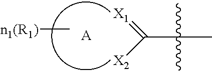

has the structure of

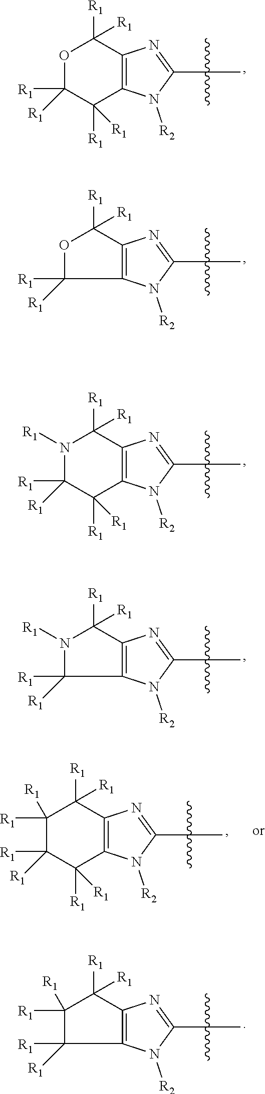

In some embodiments described herein, the substituent

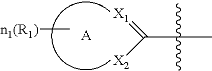

has the structure of

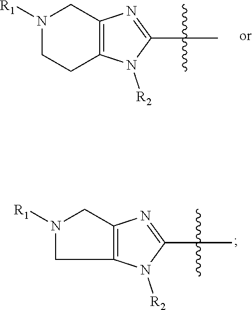

and wherein R₁ is H, NH₂, alkyl, cycloalkyl, aryl, heterocycle, or C(═O)Rₐ.

In some embodiments described herein, the substituent

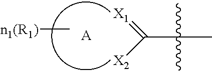

has the structure of

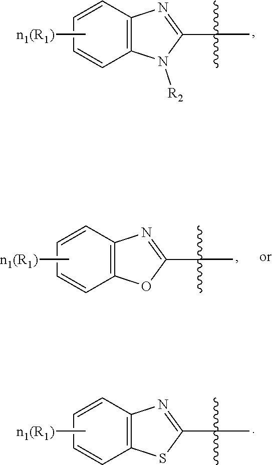

In some embodiments described herein, the substituent

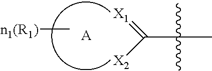

has the structure of

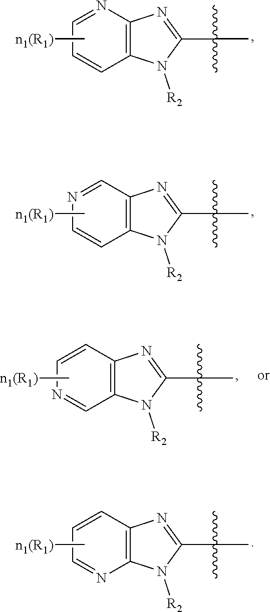

In some embodiments described herein, the substituent

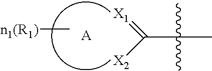

has the structure of

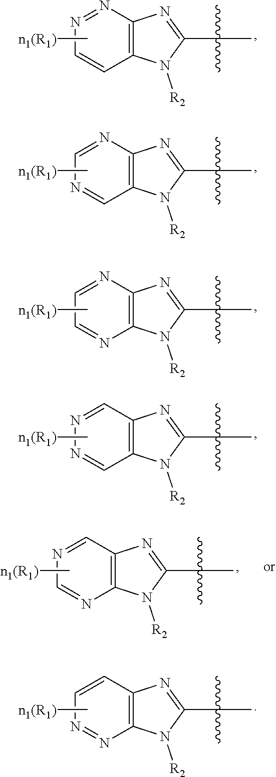

In some embodiments described herein, X is CR₁.

In some embodiments described herein, X is N.

In some embodiments described herein, Q₁, Q₂ and Q₃ are each CR₁.

In some embodiments described herein, Q₁, Q₂ and Q₃ are each N.

In some embodiments described herein, Q₁, Q₂ and Q₃ are, respectively, N, CR₁, and CR₁; or Q₁, Q₂ and Q₃ are, respectively, CR₁, N, and CR₁; or Q₁, Q₂ and Q₃ are, respectively, CR₁, CR₁, and N; or Q₁, Q₂ and Q₃ are, respectively, N, N, and CR₁; or Q₁, Q₂ and Q₃ are, respectively, N, CR₁, and N; or Q₁, Q₂ and Q₃ are, respectively, CR₁, N, and N.

In some embodiments described herein, R₃ and R₄ are each H, methyl, or ethyl.

In some embodiments described herein, R₃ and R₄ are each H.

In some embodiments described herein, Z is S.

In some embodiments described herein, Z is O.

In some embodiments described herein, Z is CR₃R₄.

In some embodiments described herein, Z is CH₂, CHMe, or CMe₂.

In some embodiments described herein, Z is NR₂.

In some embodiments described herein, Z is NH or NMe.

In some embodiments described herein, n₂ is 0.

In some embodiments described herein, n₂ is 1.

In some embodiments described herein, R₅ and R₆ are, respectively, H and H; or R₅ and R₆ are, respectively, H and Me; or R₅ and R₆ are, respectively, Me and Me.

In some embodiments described herein, B is cycloalkyl, saturated heterocycle, 5-membered heteroaryl, 6-membered aryl, 6-membered heteroaryl, fused bicyclic aryl or heteroaryl, fused tricyclic aryl or heteroaryl, aryl-aryl, heteroaryl-aryl, heteroaryl-heteroaryl, or aryl-heteroaryl, each of which is optionally substituted by one or more R₁.

In some embodiments described herein, B is optionally substituted cyclohexyl, 4-morpholinyl, N-methylpyperizinyl, thiazolyl, thiadiazolyl, oxyzolyl, pyrrolyl, pyrozolyl, phenyl, pyridyl, phenyl-thiazolyl, phenyl-thiadiazolyl, pyridinyl-thiazolyl, pyridinyl-thiadiazolyl, benzothiazolyl, pyrimidinyl, phenyl-oxadiazolyl, or thiazolopyridinyl.

In some embodiments described herein, the compound has the structure of Formula IIa or IIb,

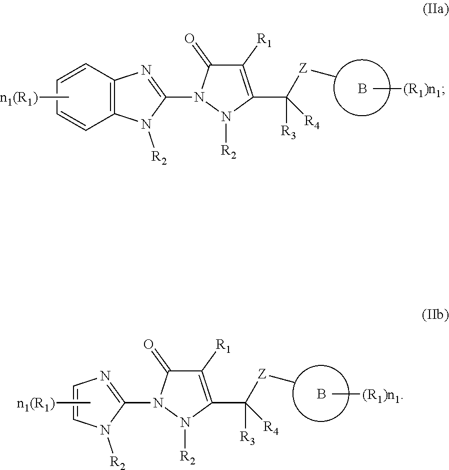

In some embodiments described herein, the compound has the structure of Formula IIIa or IIIb,

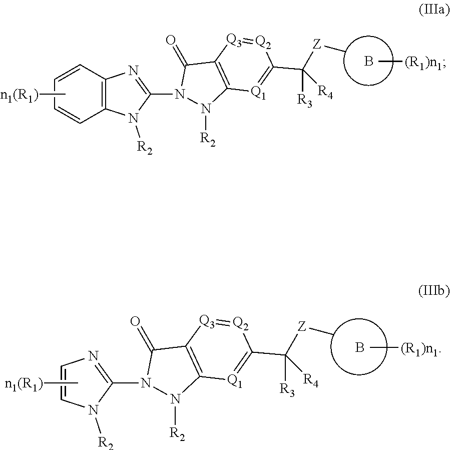

In some embodiments described herein, one or more occurrences of R₁ are independently H, Me, Et, F, Cl, Br, CF₃, OH, NH₂, Ph, or pyridinyl.

In some embodiments described herein, one or more occurrences of R₁ are independently H, F, Cl or methyl.

In some embodiments described herein, R₁ is H.

In some embodiments described herein, each occurrence of R₂ is independently H, alkyl, aryl or heteroaryl.

In some embodiments described herein, each occurrence of R₂ is independently H, Me, Ph, or pyridinyl.

In some embodiments described herein, each occurrence of R₂ is independently H, Me, or Et.

In some embodiments described herein, R₂ is H.

In some embodiments described herein, Z is NR₂; n₂ is 0; B is absent; and a R₁ substituent is connected to Z and the R₁ substituent is S(═O)Rₐ, S(═O)₂Rₐ, S(═O)₂NR_{b}R_{c}, C(═O)ORₐ, C(═O)Rₐ, C(═O)NR_{b}R_{c}, OC(═O)Rₐ, or OC(═O)NR_{b}R_{c}.

In some embodiments described herein, NR_{b}R_{c} is a heterocycle selected from the group consisting of

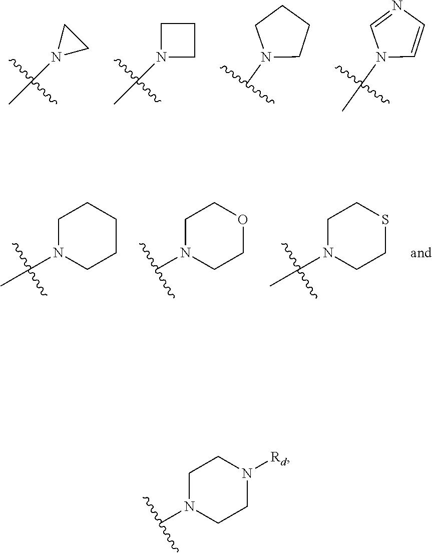

wherein R_{d} is H, Me, Et, n-Pr, i-Pr, n-Bu, i-Bu, t-Bu, CH₂CMe₃, Ph, CH₂Ph, or C(═O)(C₁-C₆ alkyl).

In some embodiments described herein, NR_{b}R_{c} is NH₂, NHMe, NMe₂, NEt₂, or NH(propyl).

In some embodiments described herein,

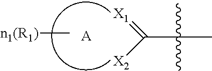

has a structure selected from the group consisting of

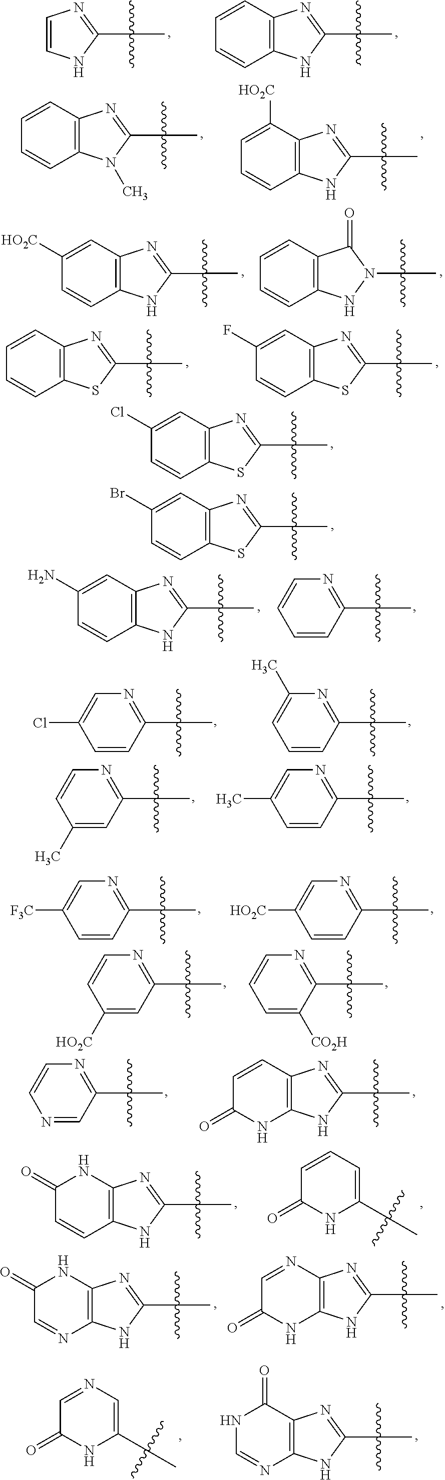

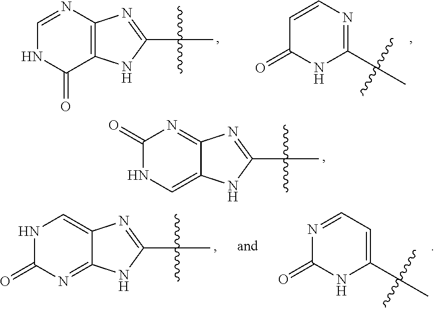 [SMILES: *c1nc2nc[nH]c(=O)c2[nH]1 ; *c1nccc(=O)[nH]1 ; *c1nc2nc(=O)[nH]cc2[nH]1 ; *c1nc2c[nH]c(=O)nc2[nH]1 ; *c1ccnc(=O)[nH]1]

In some embodiments described herein,

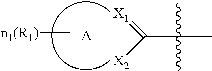

has a structure selected from the group consisting of

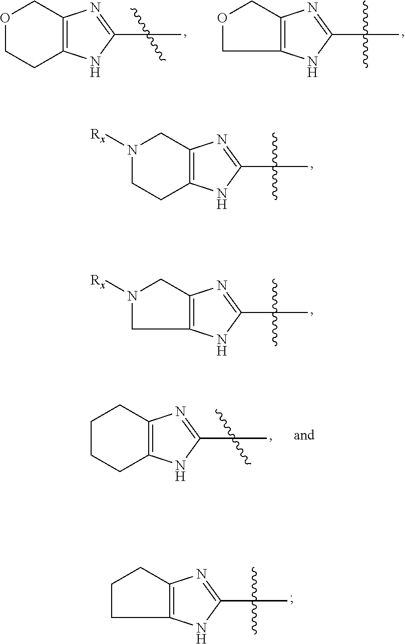

and wherein Rₓ is aryl, heteroaryl, cycloalkyl, saturated heterocycle, or C(═O)Rₐ.

In some embodiments described herein, B has a structure selected from the group consisting of

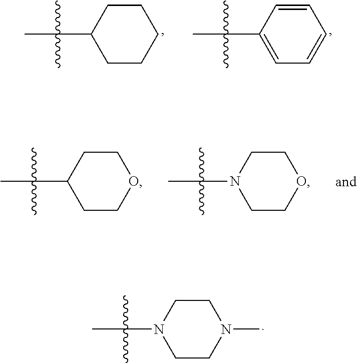

In some embodiments described herein, B has a structure selected from the group consisting of

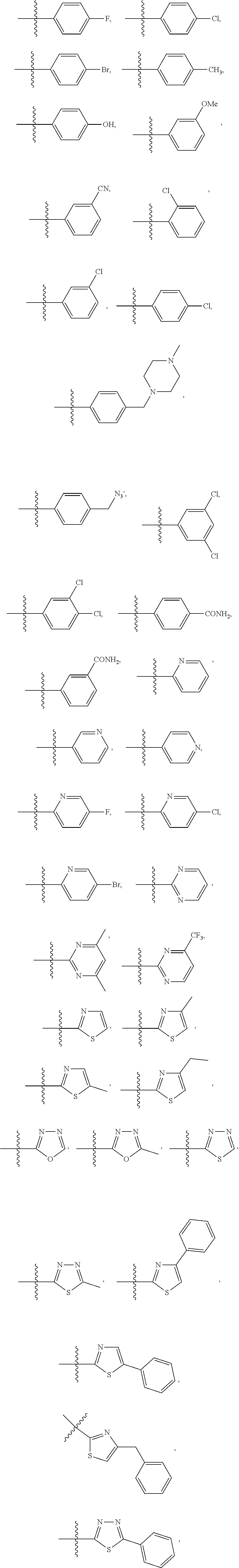

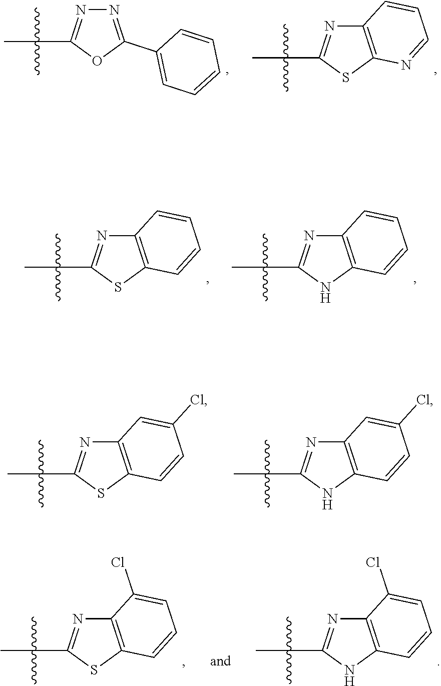

In some embodiments described herein, substituent

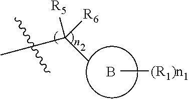

has the structure of

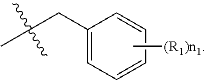

In some embodiments described herein, the compound is selected from a group consisting of:

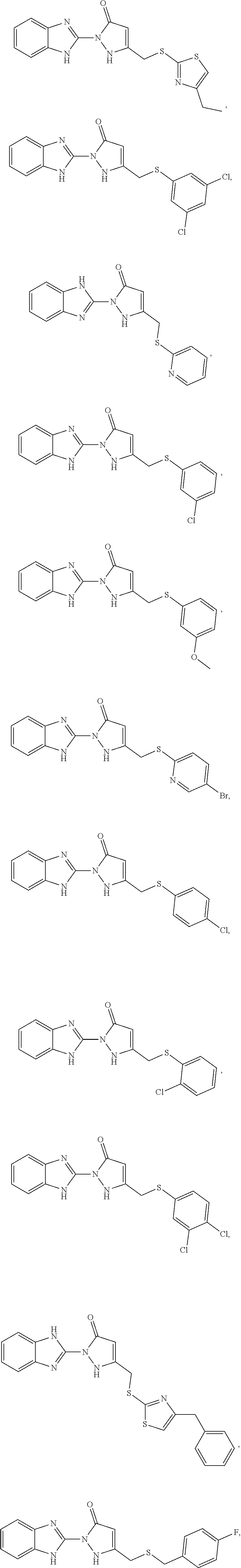

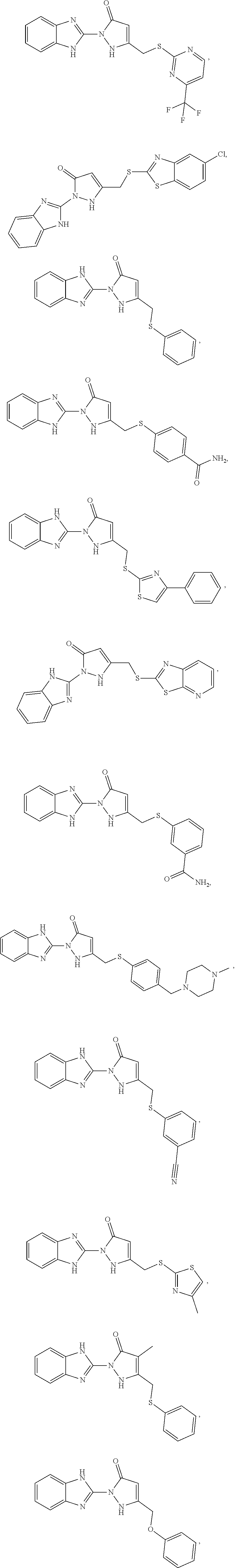

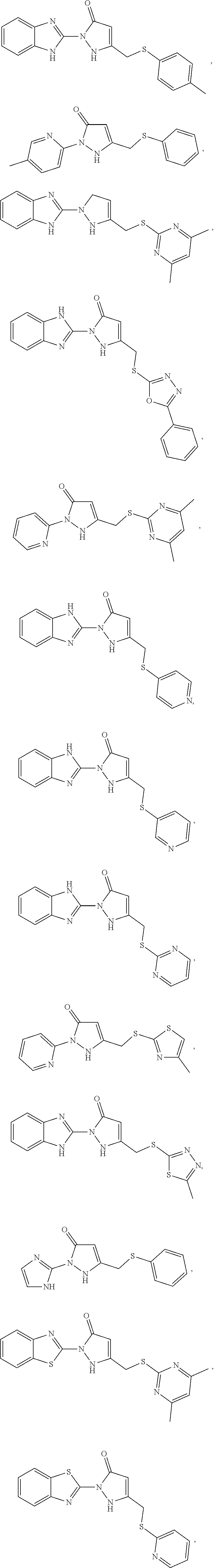

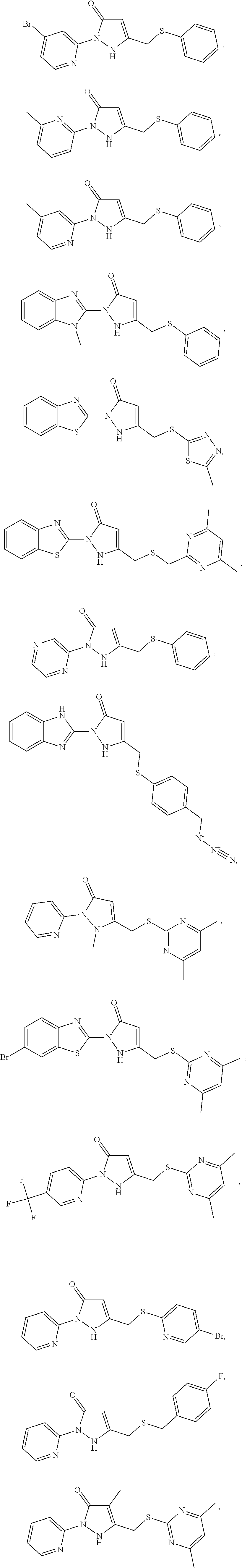

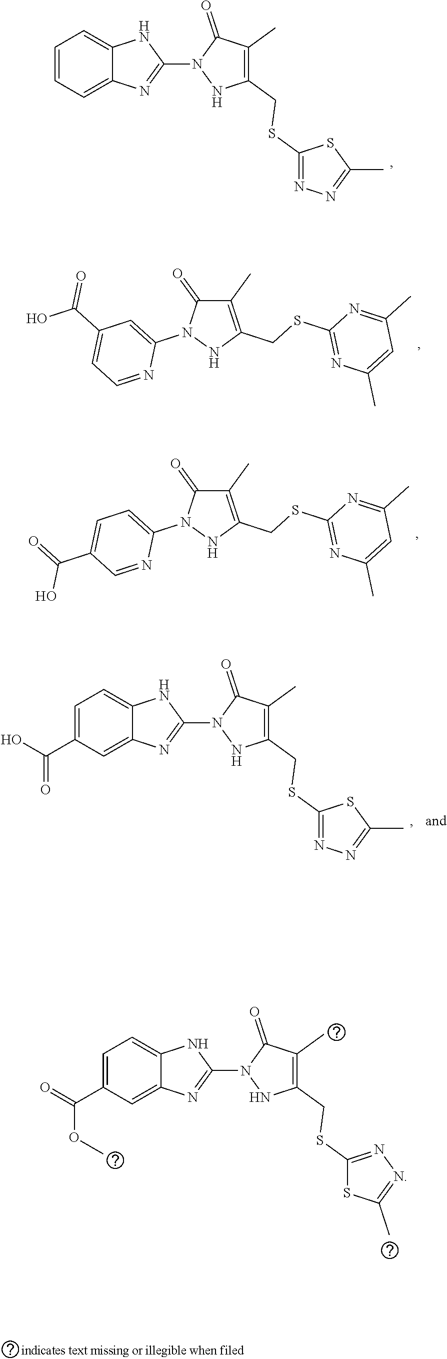 [SMILES: Cc1nnc(SCc2[nH]n(-c3nc4ccccc4[nH]3)c(=O)c2C)s1 ; Cc1cc(C)nc(SCc2[nH]n(-c3cc(C(=O)O)ccn3)c(=O)c2C)n1 ; Cc1cc(C)nc(SCc2[nH]n(-c3ccc(C(=O)O)cn3)c(=O)c2C)n1 ; Cc1nnc(SCc2[nH]n(-c3nc4cc(C(=O)O)ccc4[nH]3)c(=O)c2C)s1 ; COC(=O)c1ccc2[nH]c(-n3[nH]c(CSc4nnc(C)s4)c(C)c3=O)nc2c1]

In some embodiments described herein, the compound is selected from the group consisting of Compounds 1-136 in Tables 1-4.

In another aspect, a pharmaceutical composition is described, including at least one compound according to any one of the embodiments disclosed herein and a pharmaceutically acceptable carrier or diluent.

In yet another aspect, a method of treating cancer in a mammalian species in need thereof is described, including administering to the mammalian species a therapeutically effective amount of at least one compound according to any one of the embodiments disclosed herein.

In some embodiments described herein, the mammalian species is human.

In some embodiments described herein, the cancer is selected from the group consisting of biliary tract cancer, brain cancer, breast cancer, cervical cancer, choriocarcinoma, colon cancer, endometrial cancer, esophageal cancer, gastric (stomach) cancer, intraepithelial neoplasms, leukemias, lymphomas, liver cancer, lung cancer, melanoma, neuroblastomas, oral cancer, ovarian cancer, pancreatic cancer, prostate cancer, rectal cancer, renal (kidney) cancer, sarcomas, skin cancer, testicular cancer, and thyroid cancer.

In yet another aspect, a method of inhibiting Ras protein in a mammalian species in need thereof is described, including administering to the mammalian species a therapeutically effective amount of at least one compound according to any one of the embodiments disclosed herein.

In some embodiments described herein, the mammalian species is human.

Any one of the embodiments disclosed herein may be properly combined with any other embodiment disclosed herein. The combination of any one of the embodiments disclosed herein with any other embodiments disclosed herein is expressly contemplated. Specifically, the selection of one or more embodiments for one substituent group can be properly combined with the selection of one or more particular embodiments for any other substituent group. Such combination can be made in any one or more embodiments of the application described herein or any formula described herein.

### BRIEF DESCRIPTION OF THE FIGURES

FIG. 1 shows ERK kinase phosphorylation assay in non-Raf active cells for Compound 189. CCRF-CEM are human acute lymphoblastic leukemia cells. HEK293 are normal human embryonic kidney cells. A375 are human skin melanoma cells.

FIG. 2 shows thermophoresis binding assay for GDP-loaded K-Ras for Compound 189.

FIG. 3 shows K-Ras thermostability assay for Compound 189.

FIG. 4 shows nucleotide exchange rate data for Compound 189. Comparison to nucleotide exchange rate reduction with 66.7 μM of Compound 137 is also shown.

FIG. 5 shows ERK kinase phosphorylation assay in non-Raf active cells for Compound 188. CCRF-CEM are human acute lymphoblastic leukemia cells. HEK293 are normal human embryonic kidney cells. A375 are human skin melanoma cells.

FIG. 6 shows thermophoresis binding assay for GDP-loaded K-Ras for Compound 188.

FIG. 7 shows K-Ras thermostability assay for Compound 188.

FIG. 8 shows nucleotide exchange rate data for Compound 188. Comparison to nucleotide exchange rate reduction with 66.7 μM of Compound 137 is also shown.

### DETAILED DESCRIPTION OF THE INVENTION

### Definitions

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

The terms “alkyl” and “alk” refer to a straight or branched chain alkane (hydrocarbon) radical containing from 1 to 12 carbon atoms, preferably 1 to 6 carbon atoms. Exemplary “alkyl” groups include methyl, ethyl, propyl, isopropyl, n-butyl, t-butyl, isobutyl pentyl, hexyl, isohexyl, heptyl, 4,4-dimethylpentyl, octyl, 2,2,4-trimethylpentyl, nonyl, decyl, undecyl, dodecyl, and the like. The term “(C₁-C₄)alkyl” refers to a straight or branched chain alkane (hydrocarbon) radical containing from 1 to 4 carbon atoms, such as methyl, ethyl, propyl, isopropyl, n-butyl, t-butyl, and isobutyl. “Substituted alkyl” refers to an alkyl group substituted with one or more substituents, preferably 1 to 4 substituents, at any available point of attachment. Exemplary substituents include, but are not limited, to one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. In some embodiments, groups such as alkyl, cycloalkyl, alkenyl, alkynyl, cycloalkenyl, heterocycle and aryl can themselves be optionally substituted.

The term “alkenyl” refers to a straight or branched chain hydrocarbon radical containing from 2 to 12 carbon atoms and at least one carbon-carbon double bond. Exemplary such groups include ethenyl or allyl. The term “C₂-C₆ alkenyl” refers to a straight or branched chain hydrocarbon radical containing from 2 to 6 carbon atoms and at least one carbon-carbon double bond, such as ethylenyl, propenyl, 2-propenyl, (E)-but-2-enyl, (Z)-but-2-enyl, 2-methy(E)-but-2-enyl, 2-methy(Z)-but-2-enyl, 2,3-dimethy-but-2-enyl, (Z)-pent-2-enyl, (E)-pent-1-enyl, (Z)-hex-1-enyl, (E)-pent-2-enyl, (Z)-hex-2-enyl, (E)-hex-2-enyl, (Z)-hex-1-enyl, (E)-hex-1-enyl, (Z)-hex-3-enyl, (E)-hex-3-enyl, and (E)-hex-1,3-dienyl. “Substituted alkenyl” refers to an alkenyl group substituted with one or more substituents, preferably 1 to 4 substituents, at any available point of attachment. Exemplary substituents include, but are not limited, to one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and Rₑ together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted.

The term “alkynyl” refers to a straight or branched chain hydrocarbon radical containing from 2 to 12 carbon atoms and at least one carbon to carbon triple bond. Exemplary such groups include ethynyl. The term “C₂-C₆ alkynyl” refers to a straight or branched chain hydrocarbon radical containing from 2 to 6 carbon atoms and at least one carbon-carbon triple bond, such as ethynyl, prop-1-ynyl, prop-2-ynyl, but-1-ynyl, but-2-ynyl, pent-1-ynyl, pent-2-ynyl, hex-1-ynyl, hex-2-ynyl, hex-3-ynyl. “Substituted alkynyl” refers to an alkynyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted.

The term “cycloalkyl” refers to a fully saturated cyclic hydrocarbon group containing from 1 to 4 rings and 3 to 8 carbons per ring. “C₃-C₇ cycloalkyl” refers to cyclopropyl, cyclobutyl, cyclopentyl, cyclohexyl, or cycloheptyl. “Substituted cycloalkyl” refers to a cycloalkyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “cycloalkenyl” refers to a partially unsaturated cyclic hydrocarbon group containing 1 to 4 rings and 3 to 8 carbons per ring. Exemplary such groups include cyclobutenyl, cyclopentenyl, cyclohexenyl, etc. “Substituted cycloalkenyl” refers to a cycloalkenyl group substituted with one more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “aryl” refers to cyclic, aromatic hydrocarbon groups that have 1 to 5 aromatic rings, especially monocyclic or bicyclic groups such as phenyl, biphenyl or naphthyl. Where containing two or more aromatic rings (bicyclic, etc.), the aromatic rings of the aryl group may be joined at a single point (e.g., biphenyl), or fused (e.g., naphthyl, phenanthrenyl and the like). The term “fused aromatic ring” refers to a molecular structure having two or more aromatic rings wherein two adjacent aromatic rings have two carbon atoms in common. “Substituted aryl” refers to an aryl group substituted by one or more substituents, preferably 1 to 3 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, Rₑ and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include fused cyclic groups, especially fused cycloalkyl, fused cycloalkenyl, fused heterocycle, or fused aryl, where the aforementioned cycloalkyl, cycloalkenyl, heterocycle and aryl substituents can themselves be optionally substituted.

The term “biaryl” refers to two aryl groups linked by a single bond. The term “biheteroaryl” refers to two heteroaryl groups linked by a single bond. Similarly, the term “heteroaryl-aryl” refers to a heteroaryl group and an aryl group linked by a single bond and the term “aryl-heteroaryl” refers to an aryl group and a heteroaryl group linked by a single bond. In certain embodiments, the numbers of the ring atoms in the heteroaryl and/or aryl rings are used to specify the sizes of the aryl or heteroaryl ring in the substituents. For example, 5,6-heteroaryl-aryl refers to a substituent in which a 5-membered heteroaryl is linked to a 6-membered aryl group. Other combinations and ring sizes can be similarly specified.

The term “carbocycle” or “carbon cycle” refers to a fully saturated or partially saturated cyclic hydrocarbon group containing from 1 to 4 rings and 3 to 8 carbons per ring, or cyclic, aromatic hydrocarbon groups that have 1 to 5 aromatic rings, especially monocyclic or bicyclic groups such as phenyl, biphenyl or naphthyl. The term “carbocycle” encompasses cycloalkyl, cycloalkenyl, cycloalkynyl and aryl as defined hereinabove. The term “substituted carbocycle” refers to carbocycle or carbocyclic groups substituted with one or more substituents, preferably 1 to 4 substituents, at any available point of attachment. Exemplary substituents include, but are not limited to, those described above for substituted cycloalkyl, substituted cycloalkenyl, substituted cycloalkynyl and substituted aryl. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The terms “heterocycle” and “heterocyclic” refer to fully saturated, or partially or fully unsaturated, including aromatic (i.e., “heteroaryl”) cyclic groups (for example, 3 to 7 membered monocyclic, 7 to 11 membered bicyclic, or 8 to 16 membered tricyclic ring systems) which have at least one heteroatom in at least one carbon atom-containing ring. Each ring of the heterocyclic group may independently be saturated, or partially or fully unsaturated. Each ring of the heterocyclic group containing a heteroatom may have 1, 2, 3, or 4 heteroatoms selected from the group consisting of nitrogen atoms, oxygen atoms and sulfur atoms, where the nitrogen and sulfur heteroatoms may optionally be oxidized and the nitrogen heteroatoms may optionally be quaternized. (The term “heteroarylium” refers to a heteroaryl group bearing a quaternary nitrogen atom and thus a positive charge.) The heterocyclic group may be attached to the remainder of the molecule at any heteroatom or carbon atom of the ring or ring system. Exemplary monocyclic heterocyclic groups include azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like. Exemplary bicyclic heterocyclic groups include indolyl, isoindolyl, benzothiazolyl, benzoxazolyl, benzoxadiazolyl, benzothienyl, benzo[d][1,3]dioxolyl, 2,3-dihydrobenzo[b][1,4]dioxinyl, quinuclidinyl, quinolinyl, tetrahydroisoquinolinyl, isoquinolinyl, benzimidazolyl, benzopyranyl, indolizinyl, benzofuryl, benzofurazanyl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), triazinylazepinyl, tetrahydroquinolinyl and the like. Exemplary tricyclic heterocyclic groups include carbazolyl, benzidolyl, phenanthrolinyl, acridinyl, phenanthridinyl, xanthenyl and the like.

“Substituted heterocycle” and “substituted heterocyclic” (such as “substituted heteroaryl”) refer to heterocycle or heterocyclic groups substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “oxo” refers to —C(═O)— substituent group. When an oxo substituent group is attached to an aromatic group, e.g., aryl or heteroaryl, the bonds on the aromatic ring are re-arranged to satisfy the valence requirement. For instance, a pyridine with a 2-oxo substituent group may have the structure of

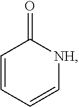

which also includes its tautomeric form of

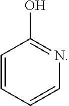

The term “alkylamino” refers to a group having the structure —NHR′, wherein R′ is hydrogen, alkyl or substituted alkyl, cycloalkyl or substituted cycloalkyl, as defined herein. Examples of alkylamino groups include, but are not limited to, methylamino, ethylamino, n-propylamino, iso-propylamino, cyclopropylamino, n-butylamino, tert-butylamino, neopentylamino, n-pentylamino, hexylamino, cyclohexylamino, and the like.

The term “dialkylamino” refers to a group having the structure —NRR′, wherein R and R′ are each independently alkyl or substituted alkyl, cycloalkyl or substituted cycloalkyl, cycloalkenyl or substituted cyclolalkenyl, aryl or substituted aryl, heterocycle or substituted heterocycle, as defined herein. R and R′ may be the same or different in a dialkyamino moiety. Examples of dialkylamino groups include, but are not limited to, dimethylamino, methyl ethylamino, diethylamino, methylpropylamino, di(n-propyl)amino, di(iso-propyl)amino, di(cyclopropyl)amino, di(n-butyl)amino, di(tert-butyl)amino, di(neopentyl)amino, di(n-pentyl)amino, di(hexyl)amino, di(cyclohexyl)amino, and the like. In certain embodiments, R and R′ are linked to form a cyclic structure. The resulting cyclic structure may be aromatic or non-aromatic. Examples of cyclic diaminoalkyl groups include, but are not limited to, aziridinyl, pyrrolidinyl, piperidinyl, morpholinyl, pyrrolyl, imidazolyl, 1,3,4-trianolyl, and tetrazolyl.

The terms “halogen” or “halo” refer to chlorine, bromine, fluorine or iodine.

The term “substituted” refers to the embodiments in which a molecule, molecular moiety or substituent group (e.g., alkyl, cycloalkyl, alkenyl, cycloalkenyl, alkynyl, heterocycle, or aryl group or any other group disclosed herein) is substituted with one or more substituents, where valence permits, preferably 1 to 6 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, alkyl, halogen-substituted alkyl, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. In the aforementioned exemplary substituents, groups such as alkyl, cycloalkyl, alkenyl, alkynyl, cycloalkenyl, heterocycle and aryl can themselves be optionally substituted. The term “optionally substituted” refers to the embodiments in which a molecule, molecular moiety or substituent group (e.g., alkyl, cycloalkyl, alkenyl, cycloalkenyl, alkynyl, heterocycle, or aryl group or any other group disclosed herein) may or may not be substituted with aforementioned one or more substituents.

Unless otherwise indicated, any heteroatom with unsatisfied valences is assumed to have hydrogen atoms sufficient to satisfy the valences.

The compounds of the present invention may form salts which are also within the scope of this invention. Reference to a compound of the present invention is understood to include reference to salts thereof, unless otherwise indicated. The term “salt(s)”, as employed herein, denotes acidic and/or basic salts formed with inorganic and/or organic acids and bases. In addition, when a compound of the present invention contains both a basic moiety, such as but not limited to a pyridine or imidazole, and an acidic moiety such as but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful, e.g., in isolation or purification steps which may be employed during preparation. Salts of the compounds of the present invention may be formed, for example, by reacting a compound described herein with an amount of acid or base, such as an equivalent amount, in a medium such as one in which the salt precipitates or in an aqueous medium followed by lyophilization.

The compounds of the present invention which contain a basic moiety, such as but not limited to an amine or a pyridine or imidazole ring, may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfonates (e.g., 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates, tartrates, thiocyanates, toluenesulfonates such as tosylates, undecanoates, and the like.

The compounds of the present invention which contain an acidic moiety, such but not limited to a carboxylic acid, may form salts with a variety of organic and inorganic bases. Exemplary basic salts include ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl) ethylenediamine), N-methyl-D-glucamines, N-methyl-D-glycamides, t-butyl amines, and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

Prodrugs and solvates of the compounds of the invention are also contemplated herein. The term “prodrug” as employed herein denotes a compound that, upon administration to a subject, undergoes chemical conversion by metabolic or chemical processes to yield a compound of the present invention, or a salt and/or solvate thereof. Solvates of the compounds of the present invention include, for example, hydrates.

Compounds of the present invention, and salts or solvates thereof, may exist in their tautomeric form (for example, as an amide or imino ether). All such tautomeric forms are contemplated herein as part of the present invention.

All stereoisomers of the present compounds (for example, those which may exist due to asymmetric carbons on various substituents), including enantiomeric forms and diastereomeric forms, are contemplated within the scope of this invention. Individual stereoisomers of the compounds of the invention may, for example, be substantially free of other isomers (e.g., as a pure or substantially pure optical isomer having a specified activity), or may be admixed, for example, as racemates or with all other, or other selected, stereoisomers. The chiral centers of the present invention may have the S or R configuration as defined by the International Union of Pure and Applied Chemistry (IUPAC) 1974 Recommendations. The racemic forms can be resolved by physical methods, such as, for example, fractional crystallization, separation or crystallization of diastereomeric derivatives or separation by chiral column chromatography. The individual optical isomers can be obtained from the racemates by any suitable method, including without limitation, conventional methods, such as, for example, salt formation with an optically active acid followed by crystallization.

Compounds of the present invention are, subsequent to their preparation, preferably isolated and purified to obtain a composition containing an amount by weight equal to or greater than 90%, for example, equal to greater than 95%, equal to or greater than 99% of the compounds (“substantially pure” compounds), which is then used or formulated as described herein. Such “substantially pure” compounds of the present invention are also contemplated herein as part of the present invention.

All configurational isomers of the compounds of the present invention are contemplated, either in admixture or in pure or substantially pure form. The definition of compounds of the present invention embraces both cis (Z) and trans (E) alkene isomers, as well as cis and trans isomers of cyclic hydrocarbon or heterocyclic rings.

Throughout the specification, groups and substituents thereof may be chosen to provide stable moieties and compounds.

Definitions of specific functional groups and chemical terms are described in more detail herein. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito (1999), the entire contents of which are incorporated herein by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

The present invention also includes isotopically labeled compounds, which are identical to the compounds disclosed herein, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention, or an enantiomer, diastereomer, tautomer, or pharmaceutically acceptable salt or solvate thereof, which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds can generally be prepared by carrying out the procedures disclosed in the Schemes and/or in the Examples below, by substituting a readily available isotopically labeled reagent for a non-isotopically labeled reagent.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valences of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of proliferative disorders. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

As used herein, the terms “cancer” and, equivalently, “tumor” refer to a condition in which abnormally replicating cells of host origin are present in a detectable amount in a subject. The cancer can be a malignant or non-malignant cancer. Cancers or tumors include, but are not limited to, biliary tract cancer; brain cancer; breast cancer; cervical cancer; choriocarcinoma; colon cancer; endometrial cancer; esophageal cancer; gastric (stomach) cancer; intraepithelial neoplasms; leukemias; lymphomas; liver cancer; lung cancer (e.g., small cell and non-small cell); melanoma; neuroblastomas; oral cancer; ovarian cancer; pancreatic cancer; prostate cancer; rectal cancer; renal (kidney) cancer; sarcomas; skin cancer; testicular cancer; thyroid cancer; as well as other carcinomas and sarcomas. Cancers can be primary or metastatic. Diseases other than cancers may be associated with mutational alternation of component of Ras signaling pathways and the compound disclosed herein may be used to treat these non-cancer diseases. Such non-cancer diseases may include: neurofibromatosis; Leopard syndrome; Noonan syndrome; Legius syndrome; Costello syndrome; Cardio-facio-cutaneous syndrome; Hereditary gingival fibromatosis type 1; Autoimmune lymphoproliferative syndrome; and capillary malformation-arterovenous malformation.

As used herein, “effective amount” refers to any amount that is necessary or sufficient for achieving or promoting a desired outcome. In some instances an effective amount is a therapeutically effective amount. A therapeutically effective amount is any amount that is necessary or sufficient for promoting or achieving a desired biological response in a subject. The effective amount for any particular application can vary depending on such factors as the disease or condition being treated, the particular agent being administered, the size of the subject, or the severity of the disease or condition. One of ordinary skill in the art can empirically determine the effective amount of a particular agent without necessitating undue experimentation.

As used herein, the term “subject” refers to a vertebrate animal. In one embodiment, the subject is a mammal or a mammalian species. In one embodiment, the subject is a human. In other embodiments, the subject is a non-human vertebrate animal, including, without limitation, non-human primates, laboratory animals, livestock, racehorses, domesticated animals, and non-domesticated animals.

### Compounds

Novel compounds as Ras protein inhibitors are described. Applicants have surprisingly discovered that the compounds disclosed herein exhibit potent Ras-inhibiting properties.

In one aspect, a compound of Formula Ia or Ib or a pharmaceutically acceptable salt thereof is described,

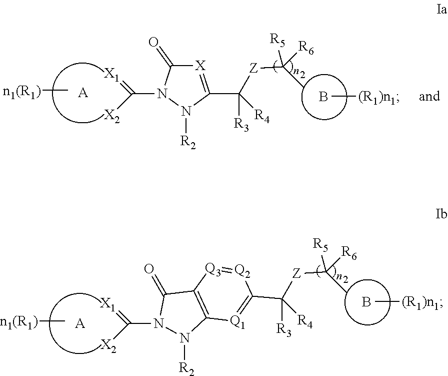

wherein

A is a monocyclic, bicyclic, or tricyclic heterocyclic group optionally substituted by one or more R₁;

each occurrence of n₁ is independently 0, 1, 2, 3, 4, 5, 6, 7, or 8;

X₁ and X₂ are each independently CR₁, O, S, N or NR₂ where valence permits; wherein at least one of X₁ and X₂ is O, N or NR₂;

each occurrence of R₁ is independently hydrogen, halogen, cyano, nitro, —N₃⁻, CF₃, OCF₃, ORₐ, optionally substituted alkyl, optionally substituted cycloalkyl, optionally substituted alkenyl, optionally substituted alkynyl, optionally substituted aryl, optionally substituted heterocycle, —CR_{b}R_{c}-(optionally substituted aryl), —CR_{b}R_{c}-(optionally substituted heteroaryl), —CR_{b}R_{c}—N₃⁻—, SRₐ, S(═O)Rₐ, S(═O)₂Rₐ, —(CR_{b}R_{c})₁₋₄—NR_{b}R_{c}, NR_{b}R_{c}, S(═O)₂NR_{b}R_{c}, oxo, C(═O)ORₐ, C(═O)Rₐ, C(═O)NR_{b}R_{c}, OC(═O)Rₐ, OC(═O)NR_{b}R_{c}, NR_{b}C(═O)ORₐ, or NR_{b}C(═O)Rₐ; or alternatively two R₁ groups substituted on the same ring taken together form an additional 3-7 membered carbocycle or heterocycle optionally substituted by one or more R_{1′};

each occurrence of R_{1′} is independently hydrogen, halogen, cyano, nitro, CF₃, OCF₃, ORₐ, optionally substituted alkyl, optionally substituted cycloalkyl, optionally substituted alkenyl, optionally substituted aryl, optionally substituted heterocycle, SRₐ, oxo, S(═O)Rₐ, S(═O)₂Rₐ, NR_{b}R_{c}, S(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₐ, or NR_{b}C(═O)Rₐ;

each occurrence of R₂ is independently hydrogen, optionally substituted alkyl, optionally substituted cycloalkyl, optionally substituted alkenyl, optionally substituted cycloalkenyl, optionally substituted alkynyl, optionally substituted heterocycle, or optionally substituted aryl;

X is CR₁ or N;

Q₁, Q₂ and Q₃ are each independently CR₁ or N;

each occurrence of R₃ and R₄ is independently hydrogen, alkyl, cycloalkyl, alkenyl, cycloalkenyl, or alkynyl; or alternatively R₃ and R₄ together with the carbon atom that they are connected to form a 3-7 membered optionally substituted carbocycle or heterocycle;

Z is CR₃R₄, NR₂, O, or S;

n₂ is 0 or 1;

each occurrence of R₅ and R₆ is independently hydrogen, alkyl, cycloalkyl, alkenyl, cycloalkenyl, or alkynyl; or alternatively R₅ and R₆ together with the carbon atom that they are connected to form a 3-7 membered optionally substituted carbocycle or heterocycle;

B is absent, or B is cycloalkyl group or saturated heterocyclic group optionally substituted by one or more R₁, or B is monocyclic, bicyclic, or tricyclic aryl or heteroaryl group optionally substituted by one or more R₁; and

Rₐ, R_{b}, and R_{c} are each independently hydrogen, alkyl, cycloalkyl, alkenyl, cycloalkenyl, alkynyl, optionally substituted heterocycle, or optionally substituted aryl; or alternatively R_{b}, and R_{R} together with the nitrogen atom that they are connected to form an optionally substituted heterocycle comprising the nitrogen atom and 0-3 additional heteroatoms each selected from the group consisting of N, O, and S.

In some embodiments, a compound of Formula Ia

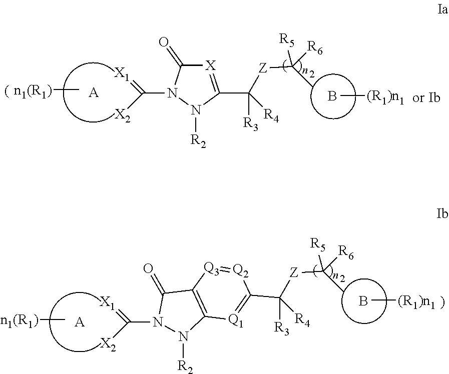

or a pharmaceutically acceptable salt thereof is described, wherein the various substituents are as defined herein, with the proviso that the compound is not

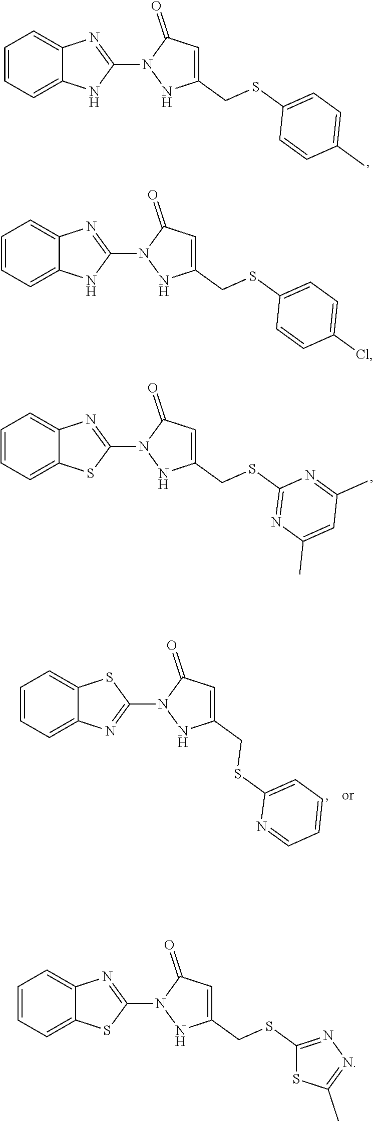 [SMILES: Cc1ccc(SCc2cc(=O)n(-c3nc4ccccc4[nH]3)[nH]2)cc1 ; O=c1cc(CSc2ccc(Cl)cc2)[nH]n1-c1nc2ccccc2[nH]1 ; Cc1cc(C)nc(SCc2cc(=O)n(-c3nc4ccccc4s3)[nH]2)n1 ; O=c1cc(CSc2ccccn2)[nH]n1-c1nc2ccccc2s1 ; Cc1nnc(SCc2cc(=O)n(-c3nc4ccccc4s3)[nH]2)s1]

In some embodiments, a compound of Formula Ia

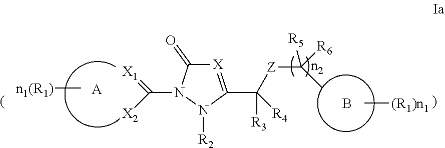

or a pharmaceutically acceptable salt thereof is described, wherein the various substituents are as defined herein, with the proviso that the compound is not

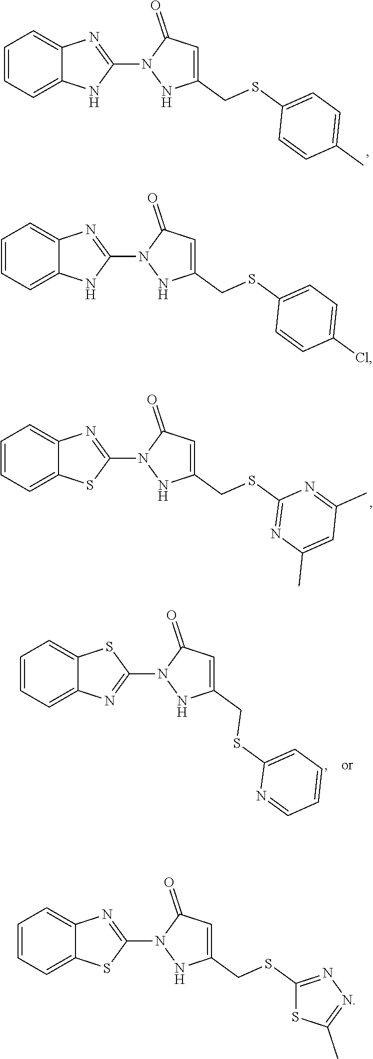 [SMILES: Cc1ccc(SCc2cc(=O)n(-c3nc4ccccc4[nH]3)[nH]2)cc1 ; O=c1cc(CSc2ccc(Cl)cc2)[nH]n1-c1nc2ccccc2[nH]1 ; Cc1cc(C)nc(SCc2cc(=O)n(-c3nc4ccccc4s3)[nH]2)n1 ; O=c1cc(CSc2ccccn2)[nH]n1-c1nc2ccccc2s1 ; Cc1nnc(SCc2cc(=O)n(-c3nc4ccccc4s3)[nH]2)s1]

In some embodiments, the compound has the structure of Formula Ia,

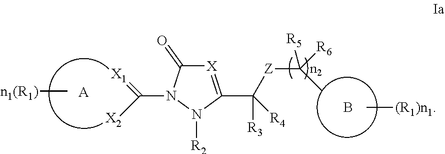

In other embodiments, the compound has the structure of Formula Ib,

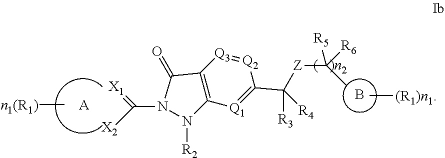

In some embodiments, X₁ and X₂ are each independently CR₁, O, S, N or NR₂ where valence permits; wherein at least one of X₁ and X₂ is O, N or NR₂. In some embodiments, X₁ is CR₁ and X₂ is N or NR₂ wherein valence permits. In some embodiments, X₁ is N and X₂ is CR₁, NR₂, O or S. In some embodiments, X₁ and X₂ are each independently N or NR₂ where valence permits. In some embodiments, X₁ is N and X₂ is NR₂. In some embodiments, X₁ is N and X₂ is S. In some embodiments, X₁ is N and X₂ is O.

A can be a monocyclic, bicyclic, or tricyclic heterocyclic group optionally substituted by one or more R₁. In some embodiments, A is an optionally substituted heterocycle selected from the group consisting of pyrrolyl, pyrazolyl, imidazolyl, isoxazolyl, thiazolyl, thiadiazolyl, isothiazolyl, pyridinyl, pyrazinyl, pyrimidinyl, pyridazinyl, benzothiazolyl and benzoimidazolyl.

In some embodiments, the substituent

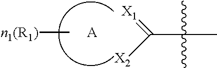

has the structure of

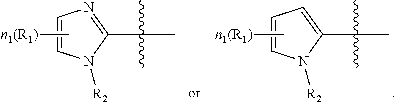

In other embodiments, the substituent

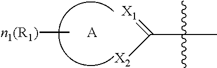

has the structure of

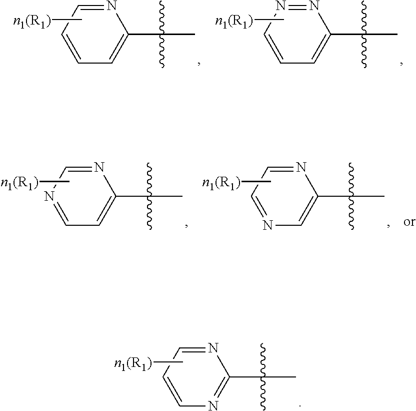

In still other embodiments, the substituent

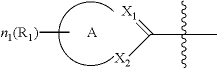

has the structure of

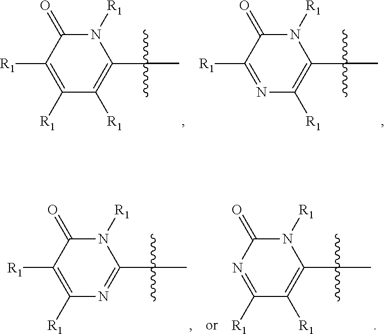

In still other embodiments, the substituent

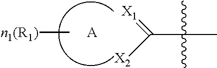

has the structure of

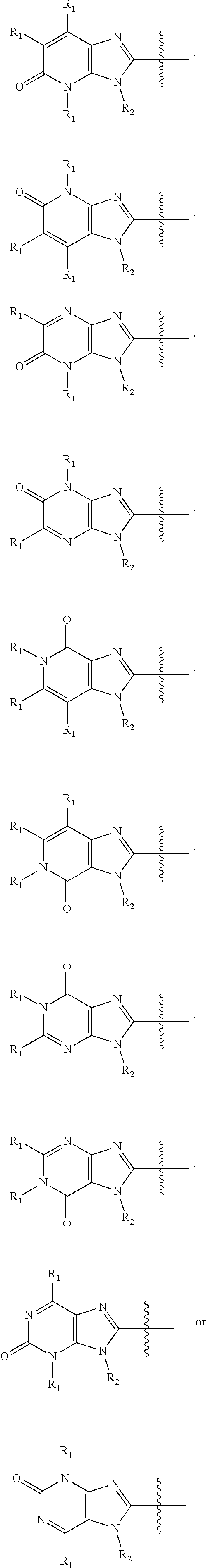

In still other embodiments, the substituent

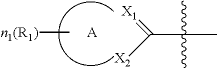

has the structure of

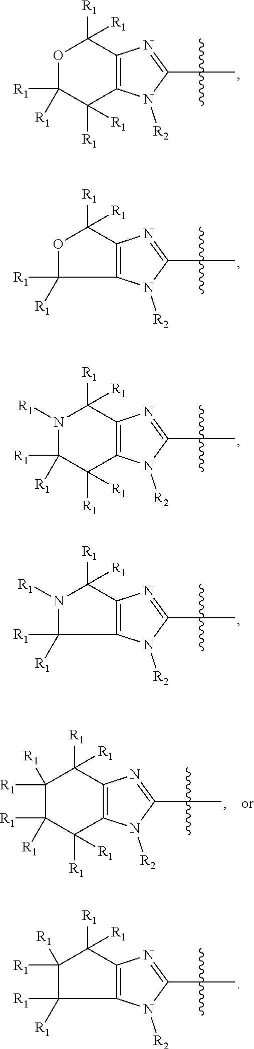

In still other embodiments, the substituent

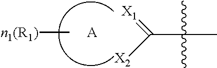

has the structure of

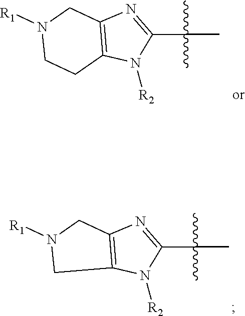

and wherein R₁ is H, NH₂, alkyl, cycloalkyl, aryl, heterocycle, or C(═O)Rₐ. In still other embodiments, the substituent

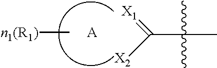

has the structure of

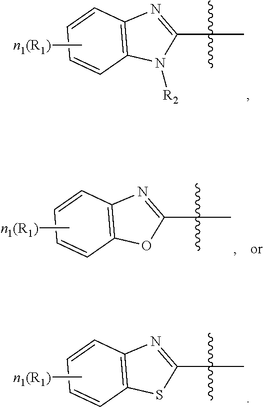

In still other embodiments, the substituent

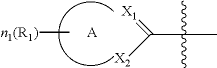

has the structure of

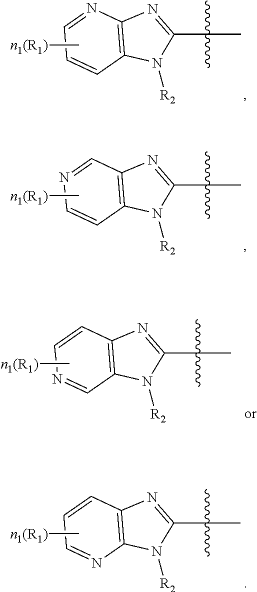

In still other embodiments, the substituent

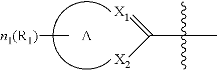

has the structure of

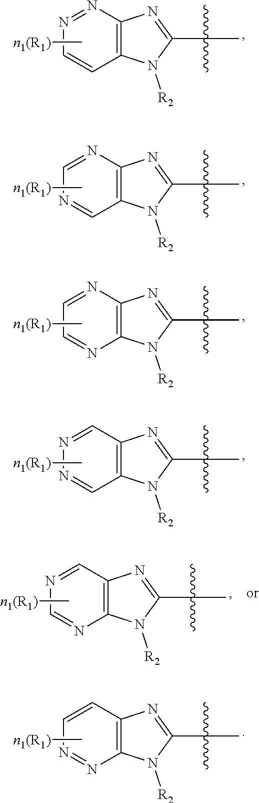

In some embodiments,

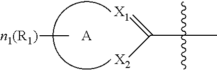

has a structure selected from the group consisting of

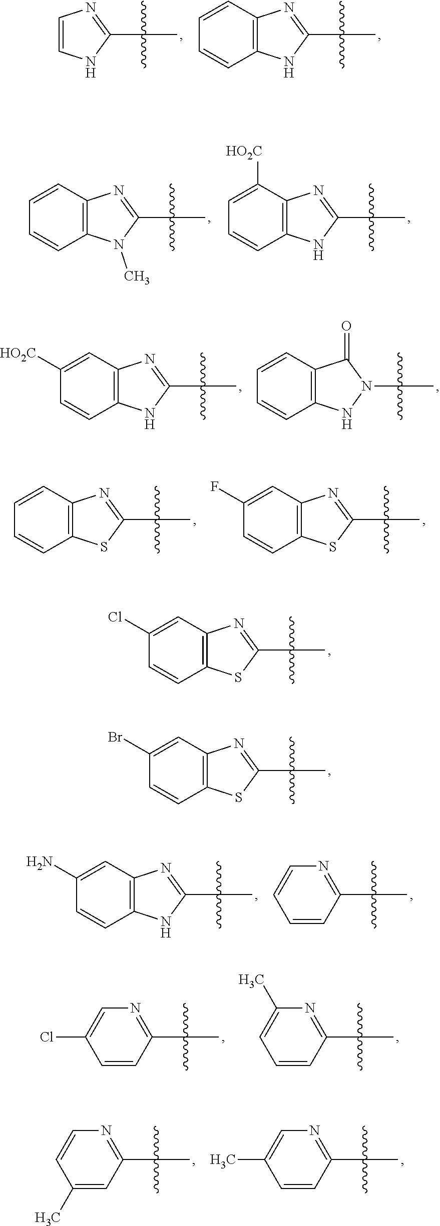

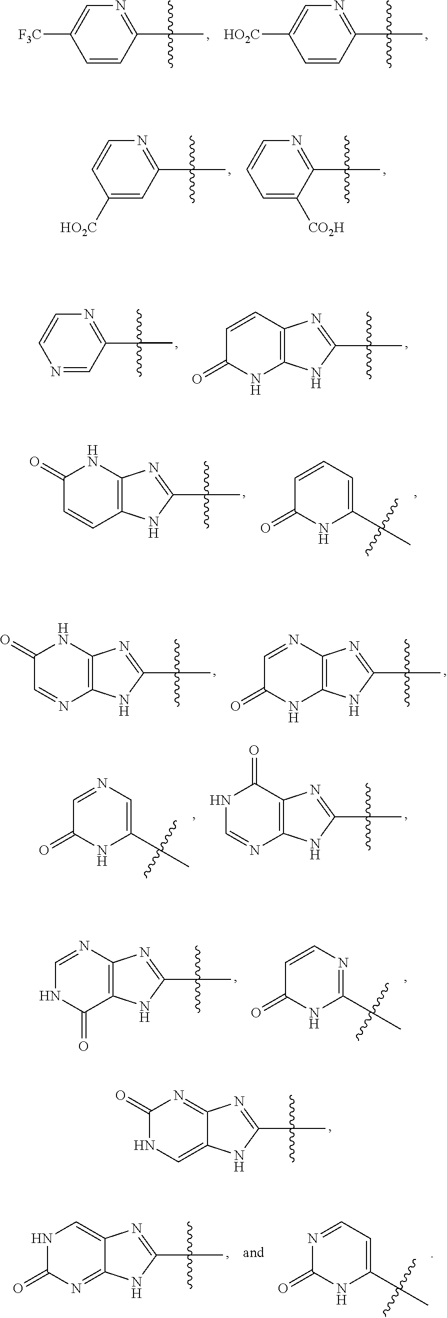

In some embodiments,

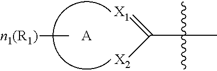

has a structure selected from the group consisting of

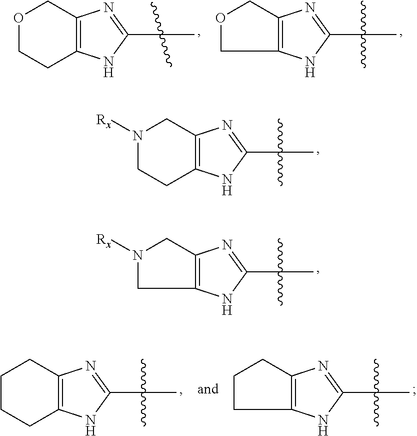

and wherein Rₓ is aryl, heteroaryl, cycloalkyl, saturated heterocycle, of C(═O)Rₐ.

In some embodiments, X is N. In other embodiments, X is CR₁. In some embodiments, X is CH, CMe, CEt, or COH. In some embodiments, X is CF, CCl, CBr, or CNH₂. In some embodiments, Q₁, Q₂ and Q₃ are each independently N or CR₁. Any combination of the embodiments for each of Q, Q₂ and Q₃ are contemplated. Thus, in some embodiments, Q₁, Q₂ and Q₃ are each CR₁. In some embodiments, Q₁, Q₂ and Q₃ are each N. In some embodiments, Q₁, Q₂ and Q₃ are, respectively, N, CR₁, and CR₁. In other embodiments, Q₁, Q₂ and Q₃ are, respectively, CR₁, N, and CR₁. In still other embodiments, Q₁, Q₂ and Q₃ are, respectively, CR₁, CR₁, and N. In still other embodiments, Q₁, Q₂ and Q₃ are, respectively, N, N, and CR₁. In still other embodiments, Q₁, Q₂ and Q₃ are, respectively, N, CR₁, and N. In still other embodiments, Q₁, Q₂ and Q₃ are, respectively, CR₁, N, and N. In certain embodiments, R₁ is H, Me, Et, F, Cl, Br, or OH.

In any of the embodiments described herein, each occurrence of R₁ is independently hydrogen, halogen, cyano, nitro, —N₃⁻—, CF₃, OCF₃, ORₐ, optionally substituted alkyl, optionally substituted cycloalkyl, optionally substituted alkenyl, optionally substituted alkynyl, optionally substituted aryl, optionally substituted heterocycle, —CR_{b}R_{c}-(optionally substituted aryl), —CR_{b}R_{c}-(optionally substituted heteroaryl), —CR_{b}R_{c}—N₃⁻—, SRₐ, S(═O)Rₐ, S(═O)₂Rₐ, —(CR_{b}R_{c})₁₋₄—NR_{b}R_{c}, NR_{b}R_{c}, S(═O)₂NR_{b}R_{c}, oxo, C(═O)ORₐ, C(═O)Rₐ, C(═O)NR_{b}R_{c}, OC(═O)Rₐ, OC(═O)NR_{b}R_{c}, NR_{b}C(═O)ORₐ, or NR_{b}C(═O)Rₐ; or alternatively two R₁ groups substituted on the same ring taken together form an additional 3-7 membered carbocycle or heterocycle optionally substituted by one or more R_{1′}. In some embodiments, each occurrence of R_{1′} is independently hydrogen, halogen, cyano, nitro, CF₃, OCF₃, ORₐ, optionally substituted alkyl, optionally substituted cycloalkyl, optionally substituted alkenyl, optionally substituted aryl, optionally substituted heterocycle, SRₐ, oxo, S(═O)Rₐ, S(═O)₂Rₐ, NR_{b}R_{c}, S(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₐ, or NR_{b}C(═O)Rₐ. In some embodiments, R_{1′} is H, Me, OH or halogen.

In any of the embodiments described herein, each occurrence of R₁ may be independently H, Me, Et, F, Cl, Br, CF₃, OH, NH₂, Ph, or pyridinyl. In some embodiments, each occurrence of R₁ is independently H, F, C₁ or methyl. In some embodiments, R₁ is H.

In other embodiments, two R₁ groups substituted on the same ring taken together form an additional, optionally substituted carbocycle or heterocycle.

In any of the embodiments described herein, each occurrence of R₂ is independently hydrogen, optionally substituted alkyl, optionally substituted cycloalkyl, optionally substituted alkenyl, optionally substituted cycloalkenyl, optionally substituted alkynyl, optionally substituted heterocycle, or optionally substituted aryl.

In any of the embodiments described herein, each occurrence of R₂ is independently H, alkyl, aryl or heteroaryl. In some embodiments, each occurrence of R₂ is independently H, Me, Ph, or 2-, 3-, or 4-pyridinyl. In other embodiments, each occurrence of R₂ is independently H, Me, or Et. In still other embodiments, R₂ is H.

In any of the embodiments described herein, each occurrence of R₃ and R₄ may be independently hydrogen, alkyl, cycloalkyl, alkenyl, cycloalkenyl, or alkynyl; or alternatively R₃ and R₄ together with the carbon atom that they are connected to form a 3-7 membered optionally substituted carbocycle or heterocycle. In some embodiments, R₃ and R₄ are each H, methyl, or ethyl. In other embodiments, R₃ and R₄ are each H. In other embodiments, R₃ and R₄ are each Me. In other embodiments, R₃ and R₄ are H and Me, respectively.

In any of the embodiments described herein, each occurrence of n₁ is independently 0, 1, 2, 3, 4, 5, 6, 7, or 8. In some embodiments, each occurrence of n₁ is independently 0, 1, 2, 3, or 4. In some embodiments, each occurrence of n₁ is independently 0, 1 or 2. In some embodiments, each occurrence of n₁ is independently 0 or 1. In some embodiments, each occurrence of n₁ is 0.

In any of the embodiments described herein, Z is CR₃R₄, NR₂, O, or S. In some embodiments, Z is S. In other embodiments, Z is O. In still other embodiments, Z is CR₃R₄. In some specific embodiments, Z is CH₂, CHMe, or CMe₂. In still other embodiments, Z is NR₂. In some specific embodiments, Z is NH or NMe.

In any of the embodiments described herein, n₂ may be 0. In some embodiments, the compound as described herein has the structure of Formula IIa or IIb,

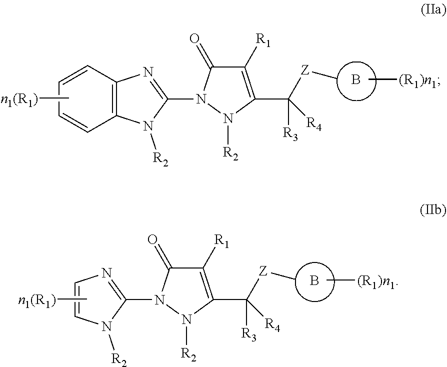

In some embodiments, the compound as described herein has structure of Formula IIIa or IIIb,

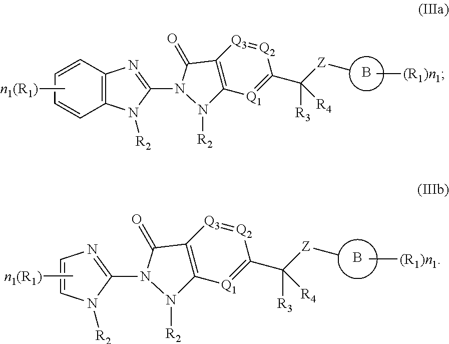

In other embodiments, n₂ is 1. In these embodiments, a compound of Formula Ia has the structure of Formula IVa:

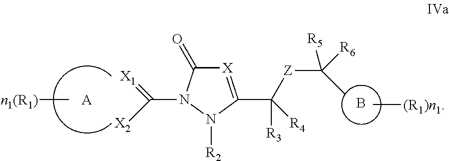

In these embodiments, a compound of Formula Ib has the structure of Formula IVb:

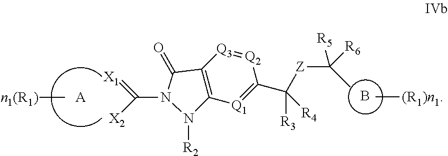

In any of the embodiments described herein, each occurrence of R₅ and R₆ may be independently hydrogen, alkyl, cycloalkyl, alkenyl, cycloalkenyl, or alkynyl; or alternatively R₅ and R₆ together with the carbon atom that they are connected to form a 3-7 membered optionally substituted carbocycle or heterocycle. In some embodiments, each occurrence of R₅ and R₆ may be H or Me. In some embodiments, R₅ and R₆ are, respectively, H and H. In some embodiments, R₅ and R₆ are, respectively, H and Me. In some embodiments, R₅ and R₆ are, respectively, Me and Me. In some embodiments, n₂ is 1 and R₅ and R₆ are each H. In other embodiments, n₂ is 1 and R₅ and R₆ are each Me. In some specific embodiments, substituent

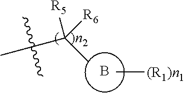

has the structure of

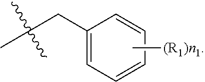

In some embodiments, B is absent. In these embodiments, a R₁ substituent may be connected to Z directly (n₂=0) or through —(CR₅R₆)— (n₂=1). That is, in the embodiments where B is absent, substituent

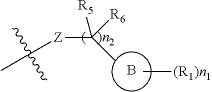

may be

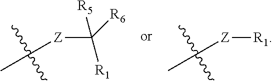

In some embodiments, B is cycloalkyl group or saturated heterocyclic group optionally substituted by one or more R₁. In some embodiments, B is monocyclic, bicyclic, or tricyclic aryl or heteroaryl group optionally substituted by one or more R₁. In other embodiments, B is cycloalkyl group optionally substituted by one or more R₁. In still other embodiments, B is saturated heterocyclic group optionally substituted by one or more R₁.

In some embodiments, B is cycloalkyl, saturated heterocycle, 5-membered heteroaryl, 6-membered aryl, 6-membered heteroaryl, fused bicyclic aryl or heteroaryl, fused tricyclic aryl or heteroaryl, aryl-aryl, heteroaryl-aryl, heteroaryl-heteroaryl, or aryl-heteroaryl, each of which is optionally substituted by one or more R₁. In some specific embodiments, B is optionally substituted cyclohexyl, 4-morpholinyl, N-methylpyperizinyl, thiazolyl, thiadiazolyl, oxyzolyl, pyrrolyl, pyrozolyl, phenyl, pyridyl, phenyl-thiazolyl, phenyl-thiadiazolyl, pyridinyl-thiazolyl, pyridinyl-thiadiazolyl, benzothiazolyl, pyrimidinyl, phenyl-oxadiazolyl, or thiazolopyridinyl.

In some embodiments, B has a structure selected from the group consisting of

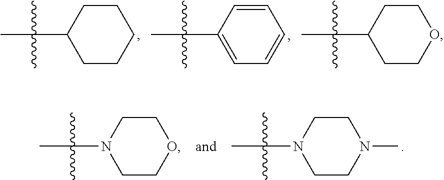

In some embodiments, B has a structure selected from the group consisting of

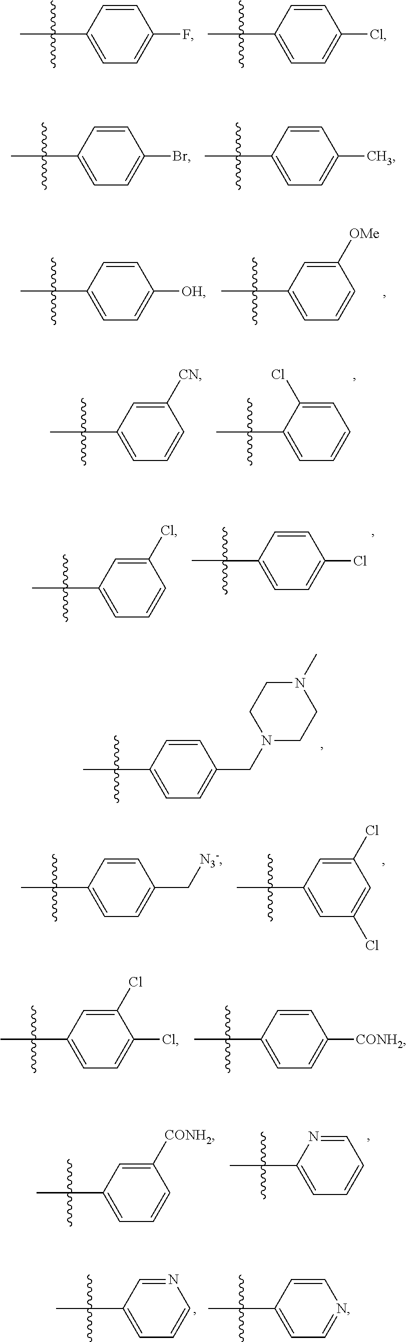

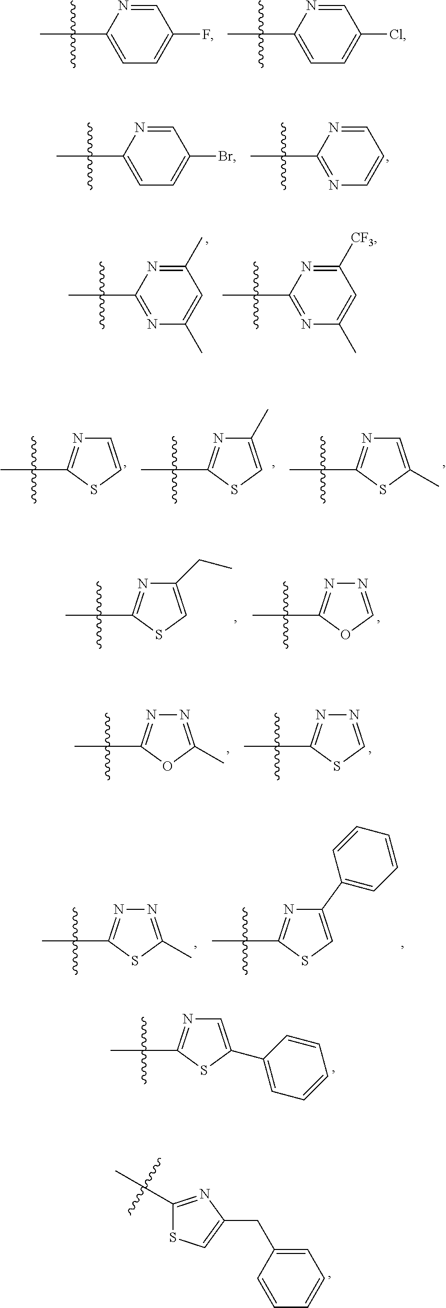

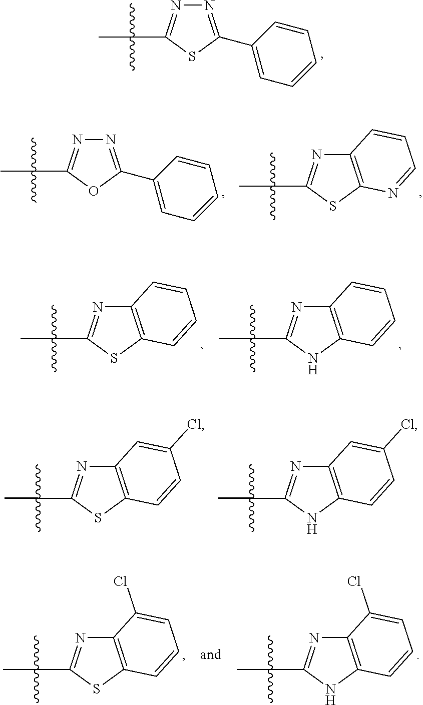

In some specific embodiments, Z is NR₂; n₂ is 0; B is absent; and the R₁ group attached to B is S(═O)Rₐ, S(═O)₂Rₐ, S(═O)₂NR_{b}R_{c}, C(═O)ORₐ, C(═O)Rₐ, C(═O)NR_{b}R_{c}, OC(═O)Rₐ, or OC(═O)NR_{b}R_{c}.

In any of the embodiments described herein, Rₐ, R_{b}, and R_{c} may each independently be hydrogen, alkyl, cycloalkyl, alkenyl, cycloalkenyl, alkynyl, optionally substituted heterocycle, or optionally substituted aryl; or alternatively R_{b}, and R_{c} together with the nitrogen atom that they are connected to form an optionally substituted heterocycle comprising the nitrogen atom and 0-3 additional heteroatoms each selected from the group consisting of N, O, and S.

In other embodiments, R_{b}, and R_{c} together with the nitrogen atom that they are connected to form an optionally substituted heterocycle comprising the nitrogen atom and 0-3 additional heteroatoms each selected from the group consisting of N, O, and S. In some embodiments, NR_{b}R_{c} is a heterocycle selected from the group consisting of

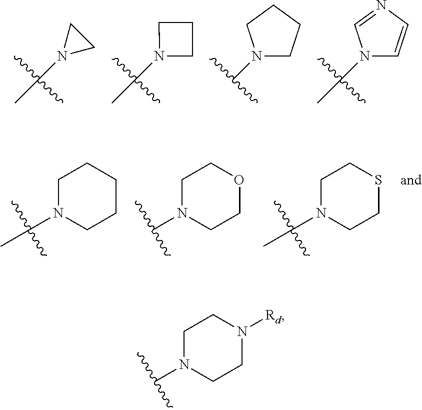

wherein R_{d} is H, Me, Et, n-Pr, i-Pr, n-Bu, i-Bu, t-Bu, CH₂CMe₃, Ph, CH₂Ph, or C(═O)(C₁-C₆ alkyl). In some embodiments, NR_{b}R_{c} is

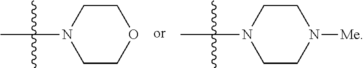 [SMILES: *N1CCOCC1 ; "or" ; *N1CCN(C)CC1]

In some embodiments, Rₐ, R_{b}, and R_{c} are each independently hydrogen, alkyl, cycloalkyl, alkenyl, cycloalkenyl, alkynyl, optionally substituted heterocycle, or optionally substituted aryl. In some embodiments, Rₐ, R_{b}, and R_{c} are each independently hydrogen, Me, Et, or propyl. In some embodiments, NR_{b}R_{c} is NH₂, NHMe, NMe₂, NEt₂, or NH(propyl).

In some embodiments, the compound is selected from a group consisting of:

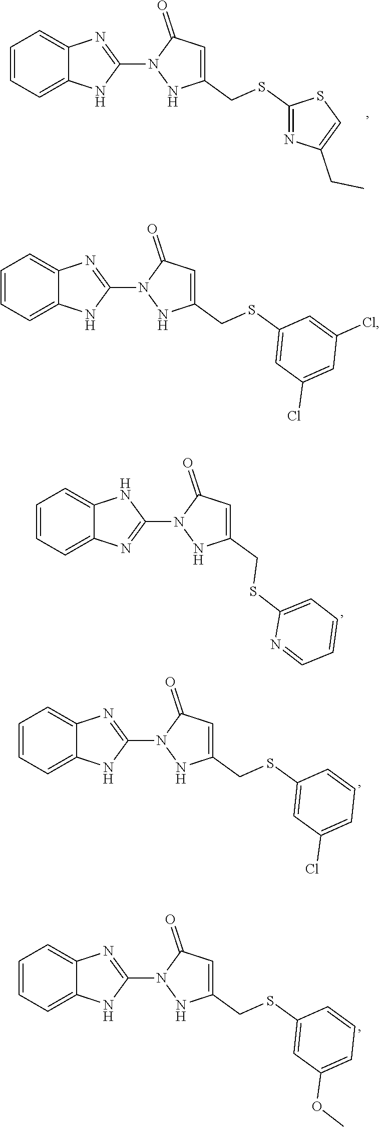 [SMILES: CCc1csc(SCc2cc(=O)n(-c3nc4ccccc4[nH]3)[nH]2)n1 ; O=c1cc(CSc2cc(Cl)cc(Cl)c2)[nH]n1-c1nc2ccccc2[nH]1 ; O=c1cc(CSc2ccccn2)[nH]n1-c1nc2ccccc2[nH]1 ; O=c1cc(CSc2cccc(Cl)c2)[nH]n1-c1nc2ccccc2[nH]1 ; COc1cccc(SCc2cc(=O)n(-c3nc4ccccc4[nH]3)[nH]2)c1]

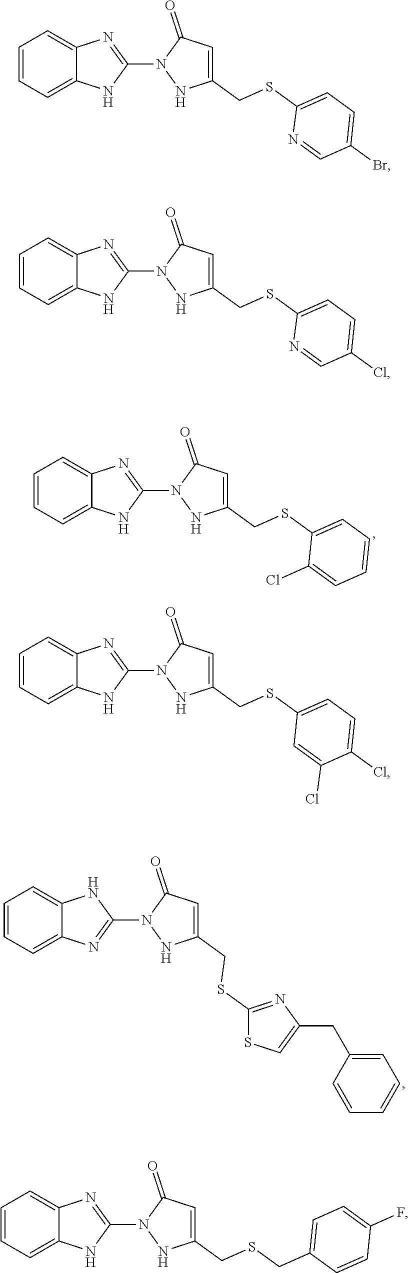 [SMILES: O=c1cc(CSc2ccc(Br)cn2)[nH]n1-c1nc2ccccc2[nH]1 ; O=c1cc(CSc2ccc(Cl)cn2)[nH]n1-c1nc2ccccc2[nH]1 ; O=c1cc(CSc2ccccc2Cl)[nH]n1-c1nc2ccccc2[nH]1 ; O=c1cc(CSc2ccc(Cl)c(Cl)c2)[nH]n1-c1nc2ccccc2[nH]1 ; O=c1cc(CSc2nc(Cc3ccccc3)cs2)[nH]n1-c1nc2ccccc2[nH]1 ; O=c1cc(CSCc2ccc(F)cc2)[nH]n1-c1nc2ccccc2[nH]1]

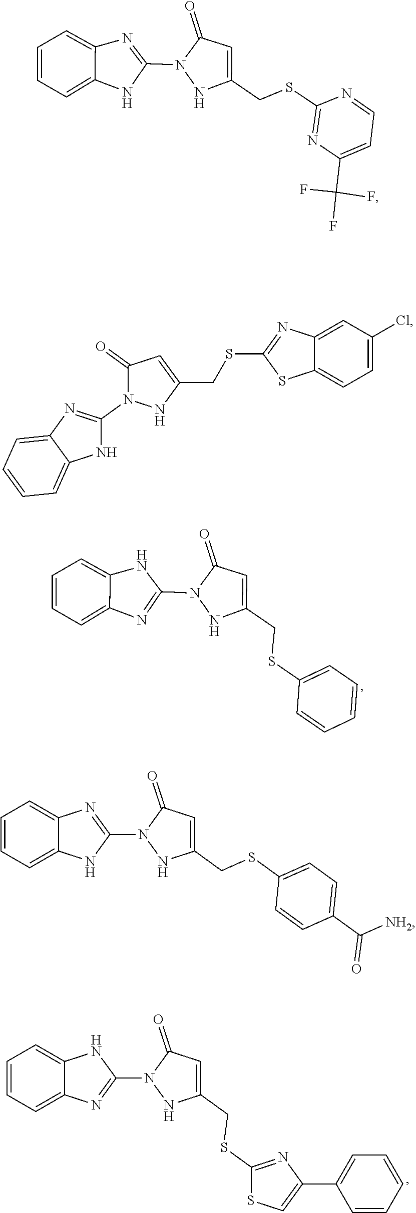 [SMILES: O=c1cc(CSc2nccc(C(F)(F)F)n2)[nH]n1-c1nc2ccccc2[nH]1 ; O=c1cc(CSc2nc3cc(Cl)ccc3s2)[nH]n1-c1nc2ccccc2[nH]1 ; O=c1cc(CSc2ccccc2)[nH]n1-c1nc2ccccc2[nH]1 ; NC(=O)c1ccc(SCc2cc(=O)n(-c3nc4ccccc4[nH]3)[nH]2)cc1 ; O=c1cc(CSc2nc(-c3ccccc3)cs2)[nH]n1-c1nc2ccccc2[nH]1]

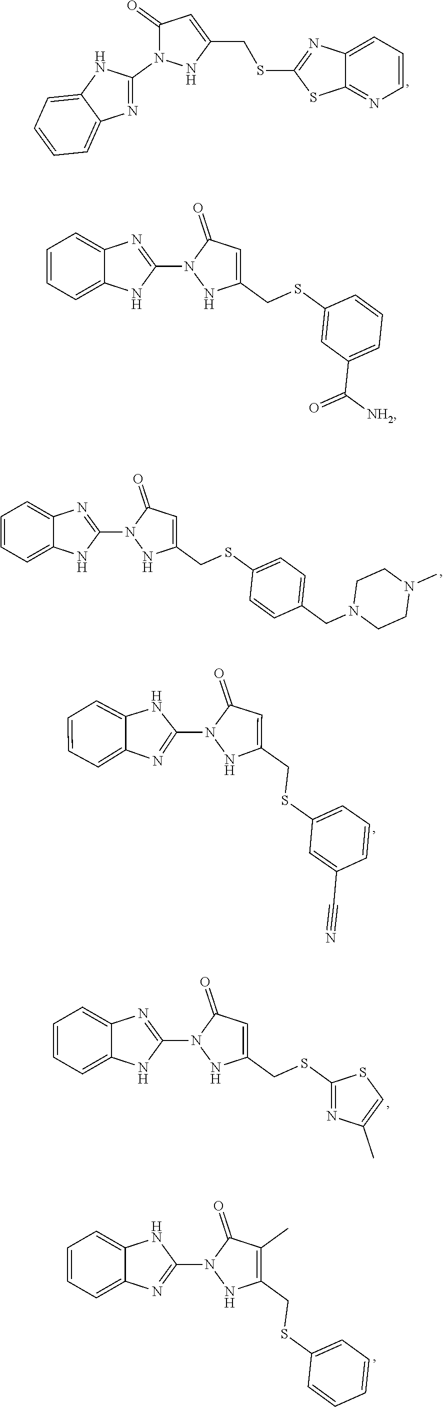

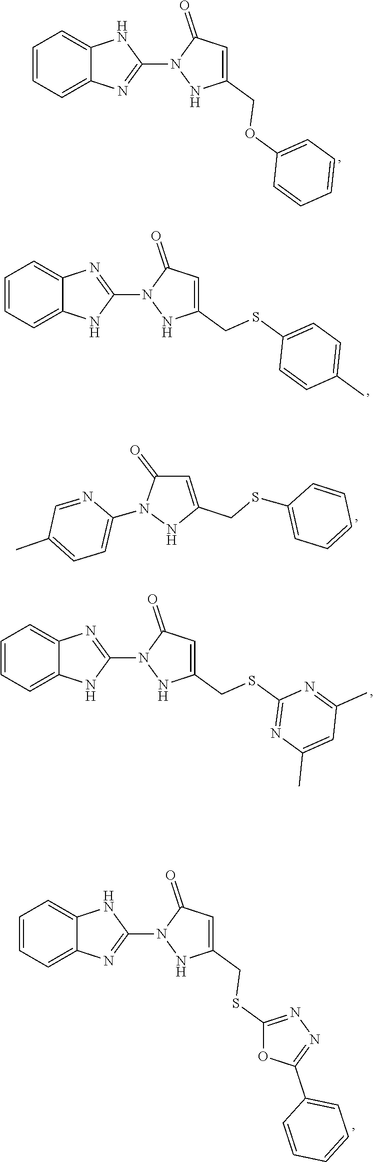

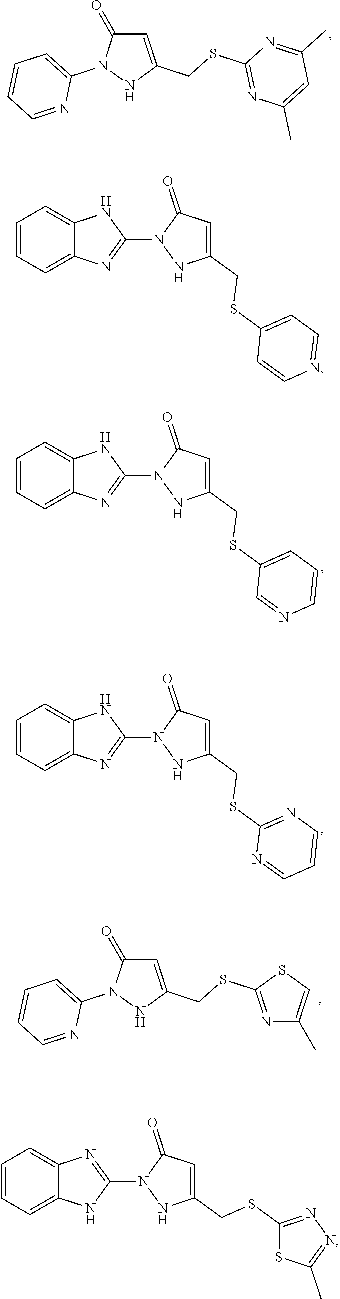 [SMILES: Cc1cc(C)nc(SCc2cc(=O)n(-c3ccccn3)[nH]2)n1 ; O=c1cc(CSc2ccncc2)[nH]n1-c1nc2ccccc2[nH]1 ; O=c1cc(CSc2cccnc2)[nH]n1-c1nc2ccccc2[nH]1 ; O=c1cc(CSc2ncccn2)[nH]n1-c1nc2ccccc2[nH]1 ; Cc1csc(SCc2cc(=O)n(-c3ccccn3)[nH]2)n1 ; Cc1nnc(SCc2cc(=O)n(-c3nc4ccccc4[nH]3)[nH]2)s1]

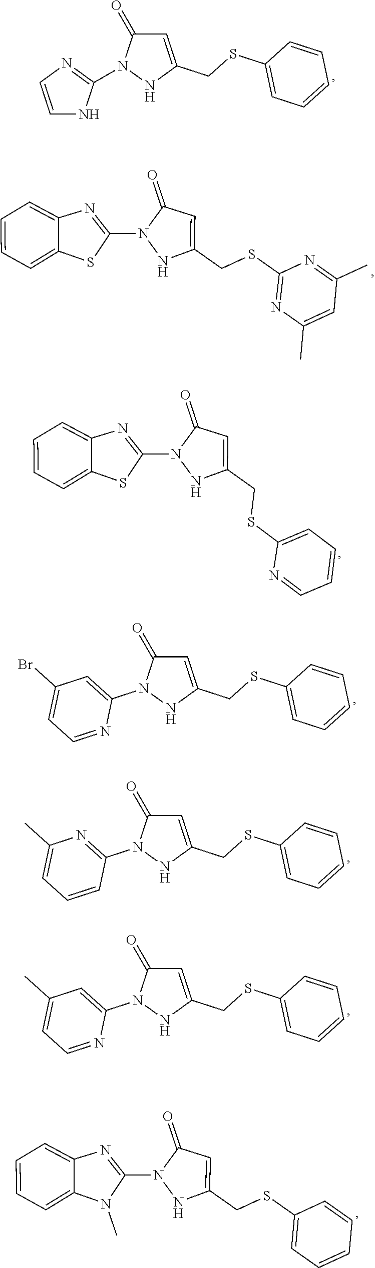

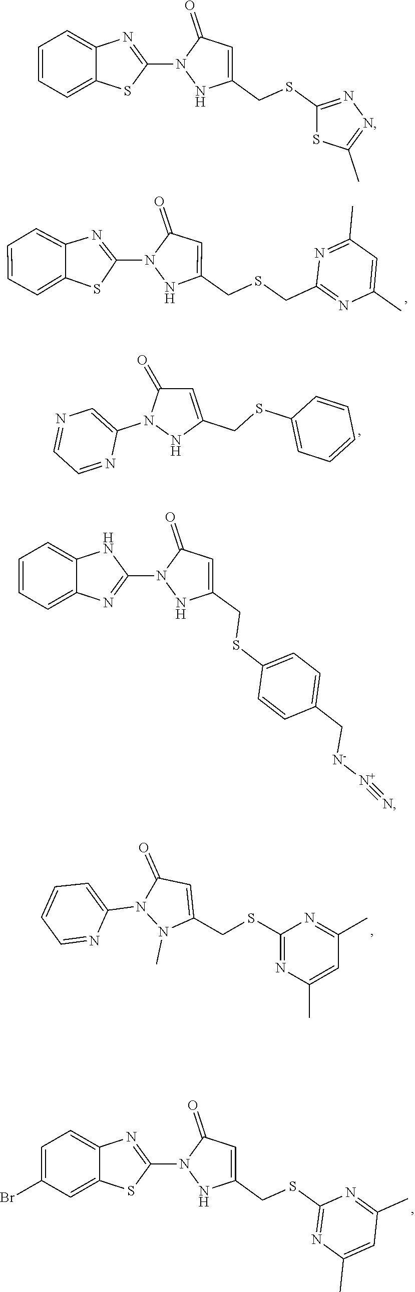

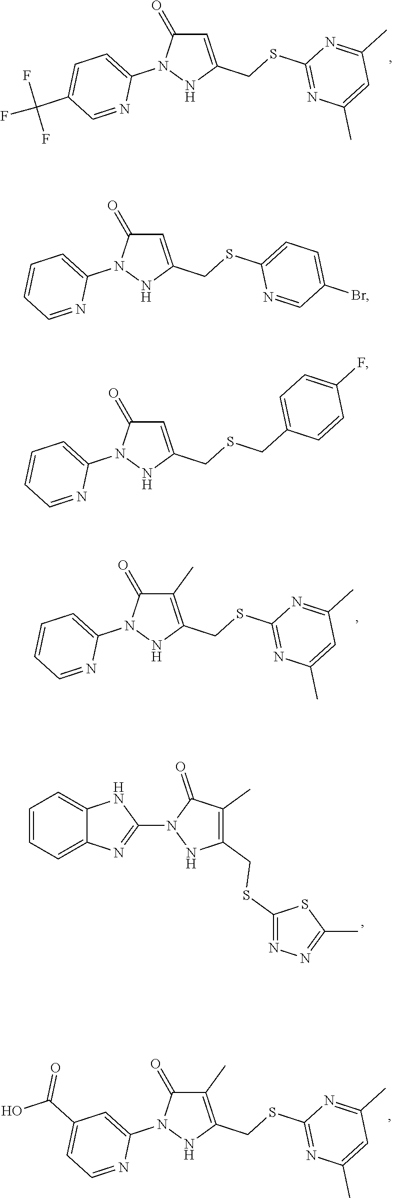 [SMILES: Cc1cc(C)nc(SCc2cc(=O)n(-c3ccc(C(F)(F)F)cn3)[nH]2)n1 ; O=c1cc(CSc2ccc(Br)cn2)[nH]n1-c1ccccn1 ; O=c1cc(CSCc2ccc(F)cc2)[nH]n1-c1ccccn1 ; Cc1cc(C)nc(SCc2[nH]n(-c3ccccn3)c(=O)c2C)n1 ; Cc1nnc(SCc2[nH]n(-c3nc4ccccc4[nH]3)c(=O)c2C)s1 ; Cc1cc(C)nc(SCc2[nH]n(-c3cc(C(=O)O)ccn3)c(=O)c2C)n1]

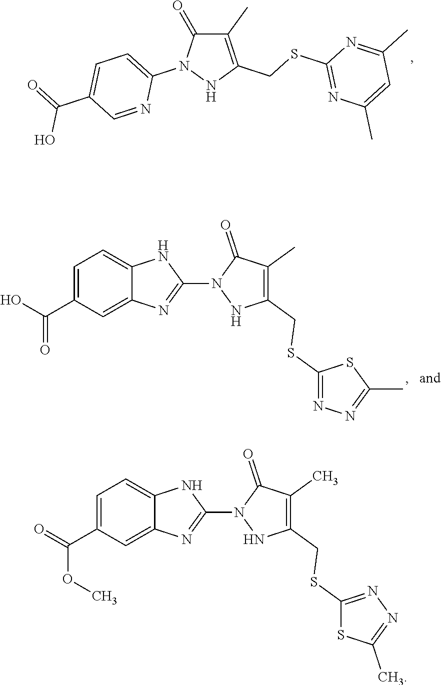 [SMILES: Cc1cc(C)nc(SCc2[nH]n(-c3ccc(C(=O)O)cn3)c(=O)c2C)n1 ; Cc1nnc(SCc2[nH]n(-c3nc4cc(C(=O)O)ccc4[nH]3)c(=O)c2C)s1 ; COC(=O)c1ccc2[nH]c(-n3[nH]c(CSc4nnc(C)s4)c(C)c3=O)nc2c1]

In yet another aspect, the present invention provides a compound of Formula Ia or Ib selected from Compounds 1 through 136 as described in Tables 1-4. The enumerated compounds in Tables 1-4 are representative and non-limiting compounds of the invention.

TABLE 1
Selected compounds of Formula Ia,

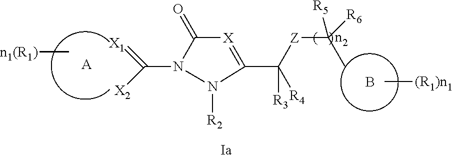

| Compound No. | 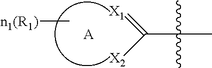 | X | R₂ | R₃, R₄ | Z | n₂ | R₅, R₆ | 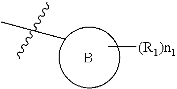 |
|---|---|---|---|---|---|---|---|---|
| 1 | 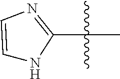 [SMILES: *c1ncc[nH]1] | N | H | H, H | O | 0 | — | 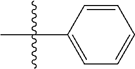 [SMILES: *c1ccccc1] |
| 2 | 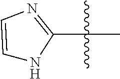 [SMILES: *c1ncc[nH]1] | N | Me | Me, Me | O | 0 | — | 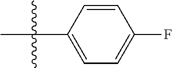 [SMILES: *c1ccc(F)cc1] |
| 3 | 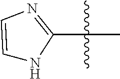 [SMILES: *c1ncc[nH]1] | N | Ph | Me, Me | S | 0 | — | 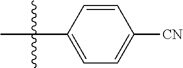 [SMILES: *c1ccc(C#N)cc1] |
| 4 | 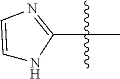 [SMILES: *c1ncc[nH]1] | CH | 4-pyridinyl | H, H | NH | 1 | H, H | 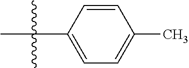 [SMILES: *c1ccc(C)cc1] |
| 5 | 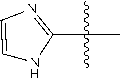 [SMILES: *c1ncc[nH]1] | CMe | Me | H, H | CH₂ | 1 | H, Me | 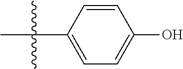 [SMILES: *c1ccc(O)cc1] |
| 6 | 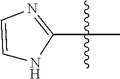 [SMILES: *c1ncc[nH]1] | C—F | H | H, H | S | 0 | — | 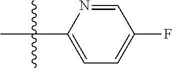 [SMILES: *c1ccc(F)cn1] |
| 7 | 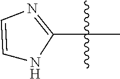 [SMILES: *c1ncc[nH]1] | C—Cl | H | H, H | S | 1 | Me, Me | 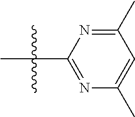 [SMILES: *c1nc(C)cc(C)n1] |
| 8 | 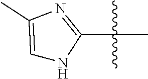 [SMILES: *c1nc(C)c[nH]1] | C—OH | Me | H, H | S | 0 | — | 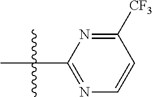 [SMILES: *c1nccc(C(F)(F)F)n1] |
| 9 | 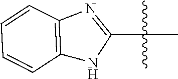 [SMILES: *c1nc2ccccc2[nH]1] | N | H | H, H | O | 0 | — | 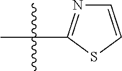 [SMILES: *c1nccs1] |
| 10 | 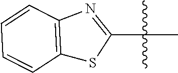 [SMILES: *c1nc2ccccc2s1] | N | Me | Me, Me | O | 0 | — | 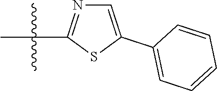 [SMILES: *c1ncc(-c2ccccc2)s1] |
| 11 | 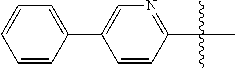 [SMILES: *c1ccc(-c2ccccc2)cn1] | N | Ph | Me, Me | S | 0 | — | 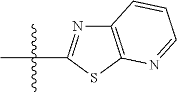 [SMILES: *c1nc2cccnc2s1] |
| 12 | 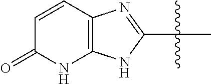 [SMILES: *c1nc2ccc(=O)[nH]c2[nH]1] | CH | 4-pyridinyl | H, H | NMe | 1 | H, H | 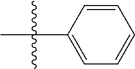 [SMILES: *c1ccccc1] |
| 13 | 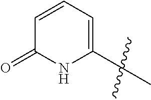 [SMILES: *c1cccc(=O)[nH]1] | CMe | Me | H, H | CH₂ | 1 | H, Me | 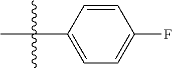 [SMILES: *c1ccc(F)cc1] |
| 14 | 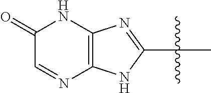 [SMILES: *c1nc2[nH]c(=O)cnc2[nH]1] | C—F | H | H, H | S | 0 | — | 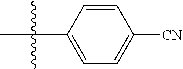 [SMILES: *c1ccc(C#N)cc1] |
| 15 | 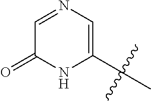 [SMILES: *c1cncc(=O)[nH]1] | C—Cl | H | H, H | S | 1 | Me, Me | 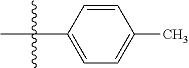 [SMILES: *c1ccc(C)cc1] |
| 16 | 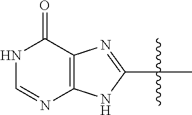 [SMILES: *c1nc2c(=O)[nH]cnc2[nH]1] | C—OH | Me | H, H | S | 0 | — | 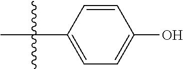 [SMILES: *c1ccc(O)cc1] |
| 17 | 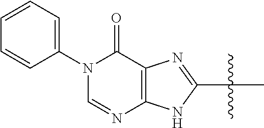 [SMILES: *c1nc2c(=O)n(-c3ccccc3)cnc2[nH]1] | N | H | H, H | O | 0 | — | 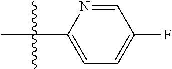 [SMILES: *c1ccc(F)cn1] |
| 18 | 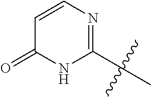 [SMILES: *c1nccc(=O)[nH]1] | N | Me | Me, Me | O | 0 | — | 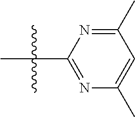 [SMILES: *c1nc(C)cc(C)n1] |
| 19 | 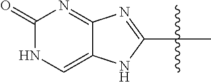 [SMILES: *c1nc2nc(=O)[nH]cc2[nH]1] | N | Ph | Me, Me | S | 0 | — | 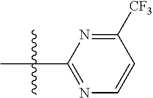 [SMILES: *c1nccc(C(F)(F)F)n1] |
| 20 | 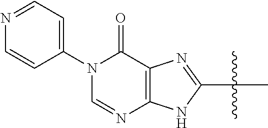 [SMILES: *c1nc2c(=O)n(-c3ccncc3)cnc2[nH]1] | N | 4-pyridinyl | H, H | NH | 1 | H, H | 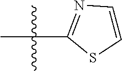 [SMILES: *c1nccs1] |
| 21 | 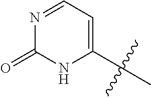 [SMILES: *c1ccnc(=O)[nH]1] | CMe | Me | H, H | CH₂ | 1 | H, Me | 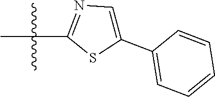 [SMILES: *c1ncc(-c2ccccc2)s1] |
| 22 | 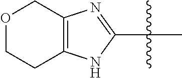 [SMILES: *c1nc2c([nH]1)CCOC2] | C—F | H | H, H | S | 0 | — | 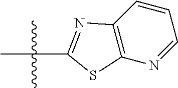 [SMILES: *c1nc2cccnc2s1] |
| 23 | 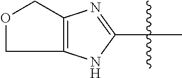 [SMILES: *c1nc2c([nH]1)COC2] | C—Cl | H | H, H | S | 1 | Me, Me | 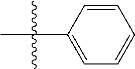 [SMILES: *c1ccccc1] |
| 24 | 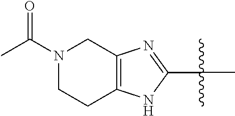 [SMILES: *c1nc2c([nH]1)CCN(C(C)=O)C2] | C—OH | Me | H, H | S | 0 | — | 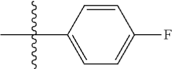 [SMILES: *c1ccc(F)cc1] |
| 25 | 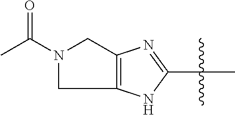 [SMILES: *c1nc2c([nH]1)CN(C(C)=O)C2] | N | H | H, H | O | 0 | — | 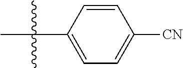 [SMILES: *c1ccc(C#N)cc1] |
| 26 | 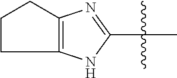 [SMILES: *c1nc2c([nH]1)CCC2] | N | Me | Me, Me | O | 0 | — | 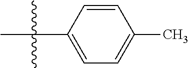 [SMILES: *c1ccc(C)cc1] |
| 27 | 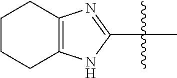 [SMILES: *c1nc2c([nH]1)CCCC2] | N | Ph | Me, Me | S | 0 | — | 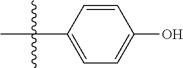 [SMILES: *c1ccc(O)cc1] |
| 28 | 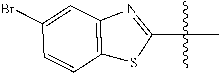 [SMILES: *c1nc2cc(Br)ccc2s1] | CH | 4-pyridinyl | H, H | NMe | 1 | H,H | 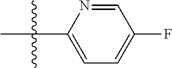 [SMILES: *c1ccc(F)cn1] |
| 29 | 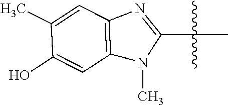 [SMILES: *c1nc2cc(C)c(O)cc2n1C] | CMe | Me | H, H | CH₂ | 1 | H, Me | 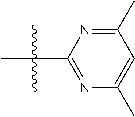 [SMILES: *c1nc(C)cc(C)n1] |
| 30 | 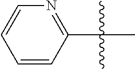 [SMILES: *c1ccccn1] | C—F | H | H, H | S | 0 | — | 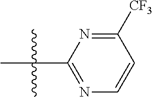 [SMILES: *c1nccc(C(F)(F)F)n1] |
| 31 | 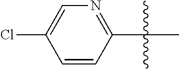 [SMILES: *c1ccc(Cl)cn1] | C—Cl | H | H, H | S | 1 | Me, Me | 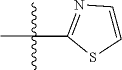 [SMILES: *c1nccs1] |
| 32 | 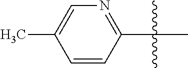 [SMILES: *c1ccc(C)cn1] | C—OH | Me | H, H | S | 0 | — | 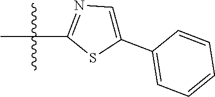 [SMILES: *c1ncc(-c2ccccc2)s1] |
| 33 | 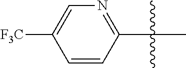 [SMILES: *c1ccc(C(F)(F)F)cn1] | CH | H | H, H | S | 0 | — | 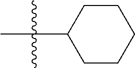 [SMILES: *C1CCCCC1] |
| 34 | 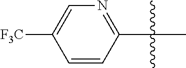 [SMILES: *c1ccc(C(F)(F)F)cn1] | CH | H | H, H | NH | 0 | — | —SO₂Me |

TABLE 2
Selected compounds of Formula Ib, wherein Q₁ = Q₂ = Q₃ = CH, i.e., compounds of Formula Ib′,

| | |
|---|---|
| | (Ib′) |
| 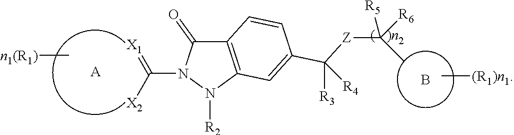 | |

| Compound No. | 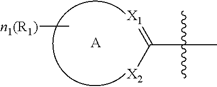 | R₂ | R₃, R₄ | Z | n₂ | R₅, R₆ | 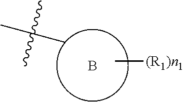 |
|---|---|---|---|---|---|---|---|
| 35 | 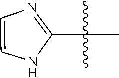 [SMILES: *c1ncc[nH]1] | H | H, H | O | 0 | — | 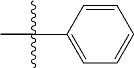 [SMILES: *c1ccccc1] |
| 36 | 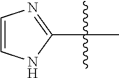 [SMILES: *c1ncc[nH]1] | Me | Me, Me | O | 0 | — | 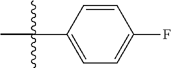 [SMILES: *c1ccc(F)cc1] |
| 37 | 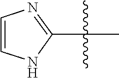 [SMILES: *c1ncc[nH]1] | Ph | Me, Me | S | 0 | — | 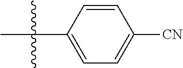 [SMILES: *c1ccc(C#N)cc1] |
| 38 | 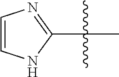 [SMILES: *c1ncc[nH]1] | 4-pyridinyl | H, H | NH | 1 | H, H | 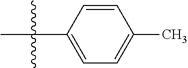 [SMILES: *c1ccc(C)cc1] |
| 39 | 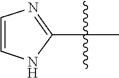 [SMILES: *c1ncc[nH]1] | Me | H, H | CH₂ | 1 | H, Me | 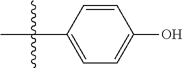 [SMILES: *c1ccc(O)cc1] |
| 40 | 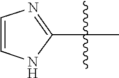 [SMILES: *c1ncc[nH]1] | H | H, H | S | 0 | — | 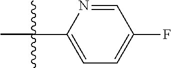 [SMILES: *c1ccc(F)cn1] |
| 41 | 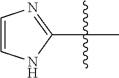 [SMILES: *c1ncc[nH]1] | H | H, H | S | 1 | Me, Me | 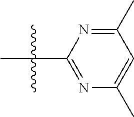 [SMILES: *c1nc(C)cc(C)n1] |
| 42 | 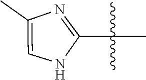 [SMILES: *c1nc(C)c[nH]1] | Me | H, H | S | 0 | — | 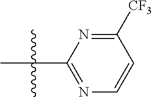 [SMILES: *c1nccc(C(F)(F)F)n1] |
| 43 | 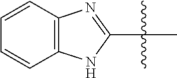 [SMILES: *c1nc2ccccc2[nH]1] | H | H, H | O | 0 | — | 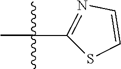 [SMILES: *c1nccs1] |
| 44 | 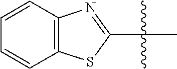 [SMILES: *c1nc2ccccc2s1] | Me | Me, Me | O | 0 | — | 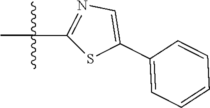 [SMILES: *c1ncc(-c2ccccc2)s1] |
| 45 | 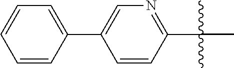 [SMILES: *c1ccc(-c2ccccc2)cn1] | Ph | Me, Me | S | 0 | — | 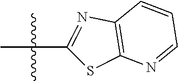 [SMILES: *c1nc2cccnc2s1] |
| 46 | 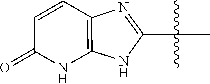 [SMILES: *c1nc2ccc(=O)[nH]c2[nH]1] | 4-pyridinyl | H, H | NMe | 1 | H, H | 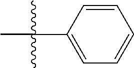 [SMILES: *c1ccccc1] |
| 47 | 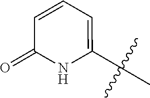 [SMILES: *c1cccc(=O)[nH]1] | Me | H, H | CH₂ | 1 | H, Me | 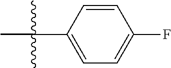 [SMILES: *c1ccc(F)cc1] |
| 48 | 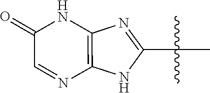 [SMILES: *c1nc2[nH]c(=O)cnc2[nH]1] | H | H, H | S | 0 | — | 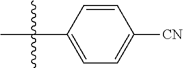 [SMILES: *c1ccc(C#N)cc1] |
| 49 | 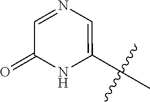 [SMILES: *c1cncc(=O)[nH]1] | H | H, H | S | 1 | Me, Me | 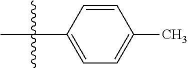 [SMILES: *c1ccc(C)cc1] |
| 50 | 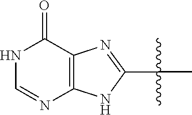 [SMILES: *c1nc2c(=O)[nH]cnc2[nH]1] | Me | H, H | S | 0 | — | 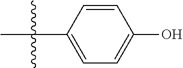 [SMILES: *c1ccc(O)cc1] |
| 51 | 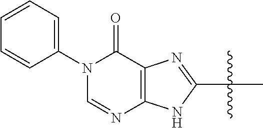 [SMILES: *c1nc2c(=O)n(-c3ccccc3)cnc2[nH]1] | H | H, H | O | 0 | — | 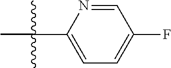 [SMILES: *c1ccc(F)cn1] |
| 52 | 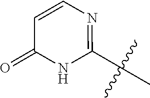 [SMILES: *c1nccc(=O)[nH]1] | Me | Me, Me | O | 0 | — | 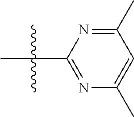 [SMILES: *c1nc(C)cc(C)n1] |
| 53 | 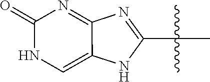 [SMILES: *c1nc2nc(=O)[nH]cc2[nH]1] | Ph | Me, Me | S | 0 | — | 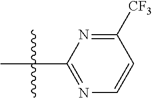 [SMILES: *c1nccc(C(F)(F)F)n1] |
| 54 | 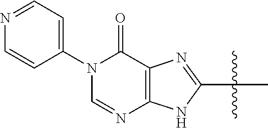 [SMILES: *c1nc2c(=O)n(-c3ccncc3)cnc2[nH]1] | 4-pyridinyl | H, H | NH | 1 | H, H | 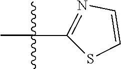 [SMILES: *c1nccs1] |
| 55 | 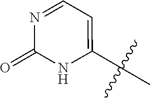 [SMILES: *c1ccnc(=O)[nH]1] | Me | H, H | CH₂ | 1 | H, Me | 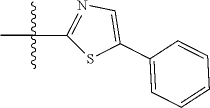 [SMILES: *c1ncc(-c2ccccc2)s1] |
| 56 | 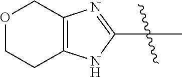 [SMILES: *c1nc2c([nH]1)CCOC2] | H | H, H | S | 0 | — | 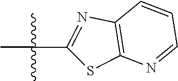 [SMILES: *c1nc2cccnc2s1] |
| 57 | 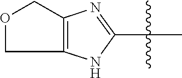 [SMILES: *c1nc2c([nH]1)COC2] | H | H, H | S | 1 | Me, Me | 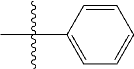 [SMILES: *c1ccccc1] |
| 58 | 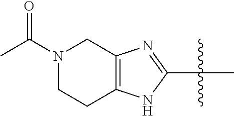 [SMILES: *c1nc2c([nH]1)CCN(C(C)=O)C2] | Me | H, H | S | 0 | — | 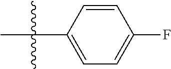 [SMILES: *c1ccc(F)cc1] |
| 59 | 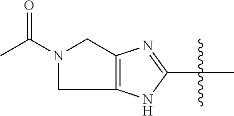 [SMILES: *c1nc2c([nH]1)CN(C(C)=O)C2] | H | H, H | O | 0 | — | 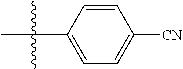 [SMILES: *c1ccc(C#N)cc1] |
| 60 | 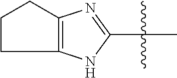 [SMILES: *c1nc2c([nH]1)CCC2] | Me | Me, Me | O | 0 | — | 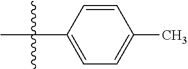 [SMILES: *c1ccc(C)cc1] |
| 61 | 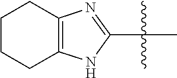 [SMILES: *c1nc2c([nH]1)CCCC2] | Ph | Me, Me | S | 0 | — | 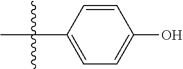 [SMILES: *c1ccc(O)cc1] |
| 62 | 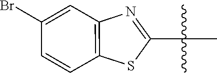 [SMILES: *c1nc2cc(Br)ccc2s1] | 4-pyridinyl | H, H | NMe | 1 | H, H | 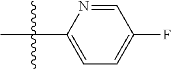 [SMILES: *c1ccc(F)cn1] |
| 63 | 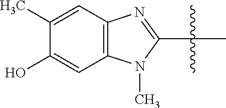 [SMILES: *c1nc2cc(C)c(O)cc2n1C] | Me | H, H | CH₂ | 1 | H, Me | 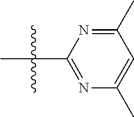 [SMILES: *c1nc(C)cc(C)n1] |
| 64 | 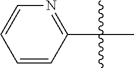 [SMILES: *c1ccccn1] | H | H, H | S | 0 | — | 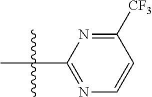 [SMILES: *c1nccc(C(F)(F)F)n1] |
| 65 | 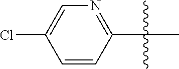 [SMILES: *c1ccc(Cl)cn1] | H | H, H | S | 1 | Me, Me | 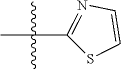 [SMILES: *c1nccs1] |
| 66 | 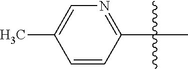 [SMILES: *c1ccc(C)cn1] | Me | H, H | S | 0 | — | 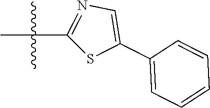 [SMILES: *c1ncc(-c2ccccc2)s1] |
| 67 | 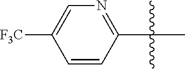 [SMILES: *c1ccc(C(F)(F)F)cn1] | H | H, H | S | 0 | — | 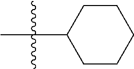 [SMILES: *C1CCCCC1] |
| 68 | 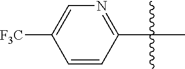 [SMILES: *c1ccc(C(F)(F)F)cn1] | H | H, H | NH | 0 | — | —SO₂Me |

TABLE 3
Selected compounds of Formula Ib, wherein Q₁ = N; and Q₂ = Q₃ = CH, i.e., Formula Ib″

| | |
|---|---|
| | (Ib″) |
| 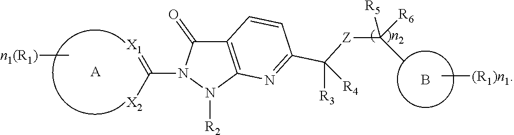 | |

| Compound No. | 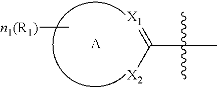 | R₂ | R₃, R₄ | Z | n₂ | R₅, R₆ | 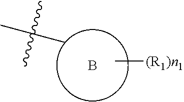 |
|---|---|---|---|---|---|---|---|
| 69 | 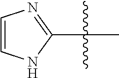 [SMILES: *c1ncc[nH]1] | H | H, H | O | 0 | — | 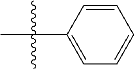 [SMILES: *c1ccccc1] |
| 70 | 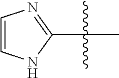 [SMILES: *c1ncc[nH]1] | Me | Me, Me | O | 0 | — | 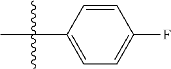 [SMILES: *c1ccc(F)cc1] |
| 71 | 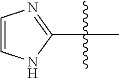 [SMILES: *c1ncc[nH]1] | Ph | Me, Me | S | 0 | — | 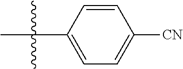 [SMILES: *c1ccc(C#N)cc1] |
| 72 | 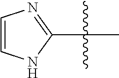 [SMILES: *c1ncc[nH]1] | 4-pyridinyl | H, H | NH | 1 | H, H | 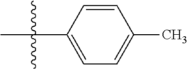 [SMILES: *c1ccc(C)cc1] |
| 73 | 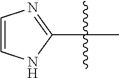 [SMILES: *c1ncc[nH]1] | Me | H, H | CH₂ | 1 | H, Me | 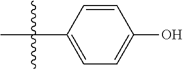 [SMILES: *c1ccc(O)cc1] |
| 74 | 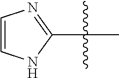 [SMILES: *c1ncc[nH]1] | H | H, H | S | 0 | — | 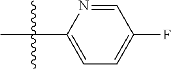 [SMILES: *c1ccc(F)cn1] |
| 75 | 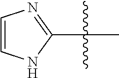 [SMILES: *c1ncc[nH]1] | H | H, H | S | 1 | Me, Me | 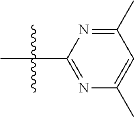 [SMILES: *c1nc(C)cc(C)n1] |
| 76 | 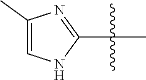 [SMILES: *c1nc(C)c[nH]1] | Me | H, H | S | 0 | — | 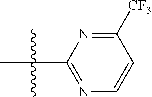 [SMILES: *c1nccc(C(F)(F)F)n1] |
| 77 | 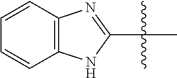 [SMILES: *c1nc2ccccc2[nH]1] | H | H, H | O | 0 | — | 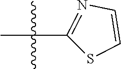 [SMILES: *c1nccs1] |
| 78 | 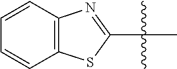 [SMILES: *c1nc2ccccc2s1] | Me | Me, Me | O | 0 | — | 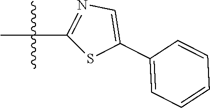 [SMILES: *c1ncc(-c2ccccc2)s1] |
| 79 | 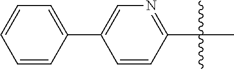 [SMILES: *c1ccc(-c2ccccc2)cn1] | Ph | Me, Me | S | 0 | — | 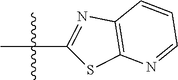 [SMILES: *c1nc2cccnc2s1] |
| 80 | 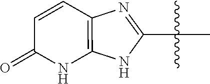 [SMILES: *c1nc2ccc(=O)[nH]c2[nH]1] | 4-pyridinyl | H, H | NMe | 1 | H, H | 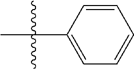 [SMILES: *c1ccccc1] |
| 81 | 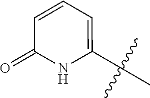 [SMILES: *c1cccc(=O)[nH]1] | Me | H, H | CH₂ | 1 | H, Me | 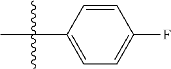 [SMILES: *c1ccc(F)cc1] |
| 82 | 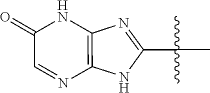 [SMILES: *c1nc2[nH]c(=O)cnc2[nH]1] | H | H, H | S | 0 | — | 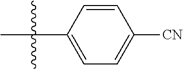 [SMILES: *c1ccc(C#N)cc1] |
| 83 | 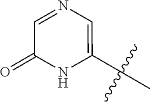 [SMILES: *c1cncc(=O)[nH]1] | H | H, H | S | 1 | Me, Me | 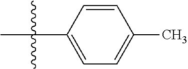 [SMILES: *c1ccc(C)cc1] |
| 84 | 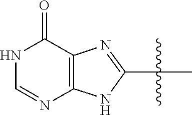 [SMILES: *c1nc2c(=O)[nH]cnc2[nH]1] | Me | H, H | S | 0 | — | 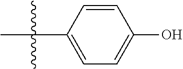 [SMILES: *c1ccc(O)cc1] |
| 85 | 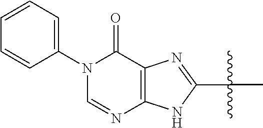 [SMILES: *c1nc2c(=O)n(-c3ccccc3)cnc2[nH]1] | H | H, H | O | 0 | — | 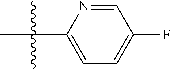 [SMILES: *c1ccc(F)cn1] |
| 86 | 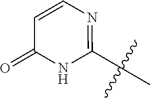 [SMILES: *c1nccc(=O)[nH]1] | Me | Me, Me | O | 0 | — | 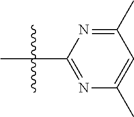 [SMILES: *c1nc(C)cc(C)n1] |
| 87 | 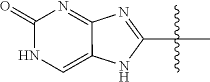 [SMILES: *c1nc2nc(=O)[nH]cc2[nH]1] | Ph | Me, Me | S | 0 | — | 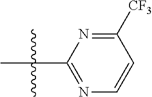 [SMILES: *c1nccc(C(F)(F)F)n1] |
| 88 | 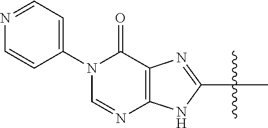 [SMILES: *c1nc2c(=O)n(-c3ccncc3)cnc2[nH]1] | 4-pyridinyl | H, H | NH | 1 | H, H | 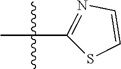 [SMILES: *c1nccs1] |
| 89 | 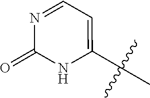 [SMILES: *c1ccnc(=O)[nH]1] | Me | H, H | CH₂ | 1 | H, Me | 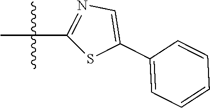 [SMILES: *c1ncc(-c2ccccc2)s1] |
| 90 | 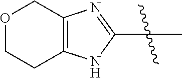 [SMILES: *c1nc2c([nH]1)CCOC2] | H | H, H | S | 0 | — | 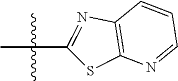 [SMILES: *c1nc2cccnc2s1] |
| 91 | 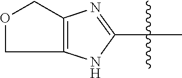 [SMILES: *c1nc2c([nH]1)COC2] | H | H, H | S | 1 | Me, Me | 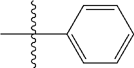 [SMILES: *c1ccccc1] |
| 92 | 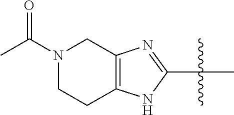 [SMILES: *c1nc2c([nH]1)CCN(C(C)=O)C2] | Me | H, H | S | 0 | — | 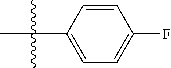 [SMILES: *c1ccc(F)cc1] |
| 93 | 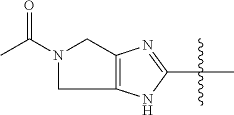 [SMILES: *c1nc2c([nH]1)CN(C(C)=O)C2] | H | H, H | O | 0 | — | 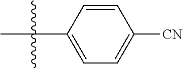 [SMILES: *c1ccc(C#N)cc1] |
| 94 | 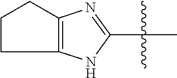 [SMILES: *c1nc2c([nH]1)CCC2] | Me | Me, Me | O | 0 | — | 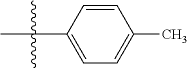 [SMILES: *c1ccc(C)cc1] |
| 95 | 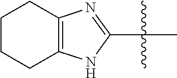 [SMILES: *c1nc2c([nH]1)CCCC2] | Ph | Me, Me | S | 0 | — | 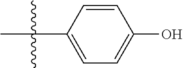 [SMILES: *c1ccc(O)cc1] |
| 96 | 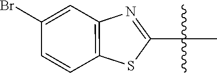 [SMILES: *c1nc2cc(Br)ccc2s1] | 4-pyridinyl | H, H | NMe | 1 | H, H | 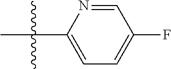 [SMILES: *c1ccc(F)cn1] |
| 97 | 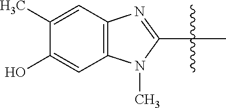 [SMILES: *c1nc2cc(C)c(O)cc2n1C] | Me | H, H | CH₂ | 1 | H, Me | 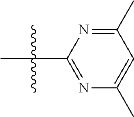 [SMILES: *c1nc(C)cc(C)n1] |
| 98 | 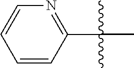 [SMILES: *c1ccccn1] | H | H, H | S | 0 | — | 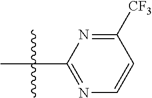 [SMILES: *c1nccc(C(F)(F)F)n1] |
| 99 | 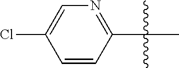 [SMILES: *c1ccc(Cl)cn1] | H | H, H | S | 1 | Me, Me | 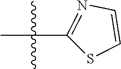 [SMILES: *c1nccs1] |
| 100 | 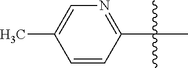 [SMILES: *c1ccc(C)cn1] | Me | H, H | S | 0 | — | 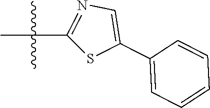 [SMILES: *c1ncc(-c2ccccc2)s1] |
| 101 | 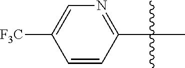 [SMILES: *c1ccc(C(F)(F)F)cn1] | H | H, H | S | 0 | — | 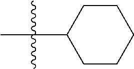 [SMILES: *C1CCCCC1] |
| 102 | 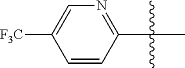 [SMILES: *c1ccc(C(F)(F)F)cn1] | H | H, H | NH | 0 | — | —SO₂Me |

TABLE 4
Selected compounds of Formula Ib, wherein Q₁ = Q₃ = N; and Q₂ = CMe, i.e., Formula Ib′′′

| | |
|---|---|
| | (Ib′′′) |
| 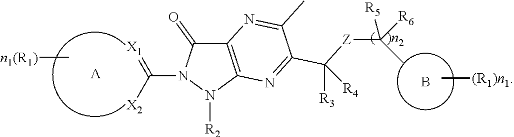 | |

| Compound No. | 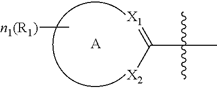 | R₂ | R₃, R₄ | Z | n₂ | R₅, R₆ | 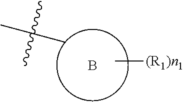 |
|---|---|---|---|---|---|---|---|
| 103 | 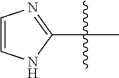 [SMILES: *c1ncc[nH]1] | H | H, H | O | 0 | — | 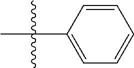 [SMILES: *c1ccccc1] |
| 104 | 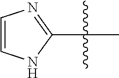 [SMILES: *c1ncc[nH]1] | Me | Me, Me | O | 0 | — | 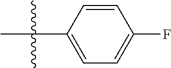 [SMILES: *c1ccc(F)cc1] |
| 105 | 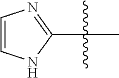 [SMILES: *c1ncc[nH]1] | Ph | Me, Me | S | 0 | — | 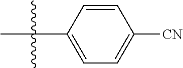 [SMILES: *c1ccc(C#N)cc1] |
| 106 | 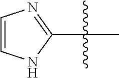 [SMILES: *c1ncc[nH]1] | 4-pyridinyl | H, H | NH | 1 | H, H | 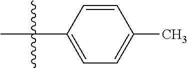 [SMILES: *c1ccc(C)cc1] |
| 107 | 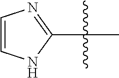 [SMILES: *c1ncc[nH]1] | Me | H, H | CH₂ | 1 | H, Me | 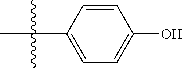 [SMILES: *c1ccc(O)cc1] |
| 108 | 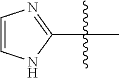 [SMILES: *c1ncc[nH]1] | H | H, H | S | 0 | — | 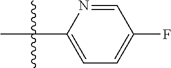 [SMILES: *c1ccc(F)cn1] |
| 109 | 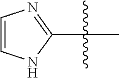 [SMILES: *c1ncc[nH]1] | H | H, H | S | 1 | Me, Me | 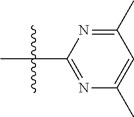 [SMILES: *c1nc(C)cc(C)n1] |
| 110 | 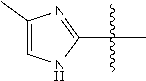 [SMILES: *c1nc(C)c[nH]1] | Me | H, H | S | 0 | — | 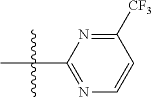 [SMILES: *c1nccc(C(F)(F)F)n1] |
| 111 | 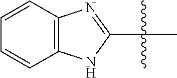 [SMILES: *c1nc2ccccc2[nH]1] | H | H, H | O | 0 | — | 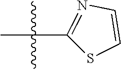 [SMILES: *c1nccs1] |
| 112 | 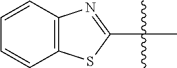 [SMILES: *c1nc2ccccc2s1] | Me | Me, Me | O | 0 | — | 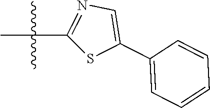 [SMILES: *c1ncc(-c2ccccc2)s1] |
| 113 | 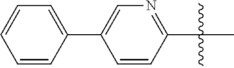 [SMILES: *c1ccc(-c2ccccc2)cn1] | Ph | Me, Me | S | 0 | — | 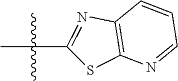 [SMILES: *c1nc2cccnc2s1] |
| 114 | 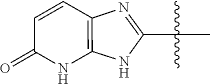 [SMILES: *c1nc2ccc(=O)[nH]c2[nH]1] | 4-pyridinyl | H, H | NMe | 1 | H, H | 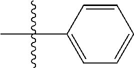 [SMILES: *c1ccccc1] |
| 115 | 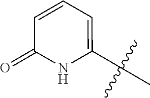 [SMILES: *c1cccc(=O)[nH]1] | Me | H, H | CH₂ | 1 | H, Me | 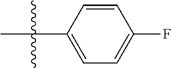 [SMILES: *c1ccc(F)cc1] |
| 116 | 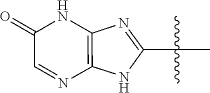 [SMILES: *c1nc2[nH]c(=O)cnc2[nH]1] | H | H, H | S | 0 | — | 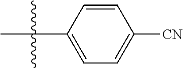 [SMILES: *c1ccc(C#N)cc1] |
| 117 | 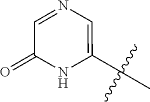 [SMILES: *c1cncc(=O)[nH]1] | H | H, H | S | 1 | Me, Me | 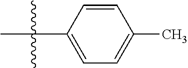 [SMILES: *c1ccc(C)cc1] |
| 118 | 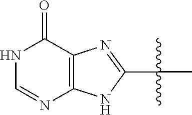 [SMILES: *c1nc2c(=O)[nH]cnc2[nH]1] | Me | H, H | S | 0 | — | 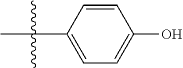 [SMILES: *c1ccc(O)cc1] |
| 119 | 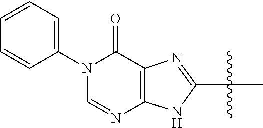 [SMILES: *c1nc2c(=O)n(-c3ccccc3)cnc2[nH]1] | H | H, H | O | 0 | — | 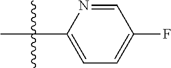 [SMILES: *c1ccc(F)cn1] |
| 120 | 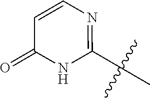 [SMILES: *c1nccc(=O)[nH]1] | Me | Me, Me | O | 0 | — | 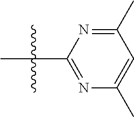 [SMILES: *c1nc(C)cc(C)n1] |
| 121 | 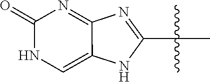 [SMILES: *c1nc2nc(=O)[nH]cc2[nH]1] | Ph | Me, Me | S | 0 | — | 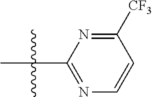 [SMILES: *c1nccc(C(F)(F)F)n1] |
| 122 | 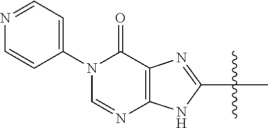 [SMILES: *c1nc2c(=O)n(-c3ccncc3)cnc2[nH]1] | 4-pyridinyl | H, H | NH | 1 | H, H | 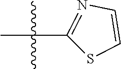 [SMILES: *c1nccs1] |
| 123 | 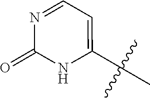 [SMILES: *c1ccnc(=O)[nH]1] | Me | H, H | CH₂ | 1 | H, Me | 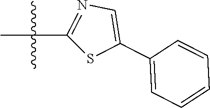 [SMILES: *c1ncc(-c2ccccc2)s1] |
| 124 | 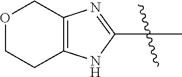 [SMILES: *c1nc2c([nH]1)CCOC2] | H | H, H | S | 0 | — | 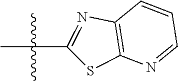 [SMILES: *c1nc2cccnc2s1] |
| 125 | 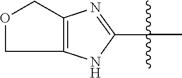 [SMILES: *c1nc2c([nH]1)COC2] | H | H, H | S | 1 | Me, Me | 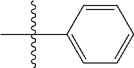 [SMILES: *c1ccccc1] |
| 126 | 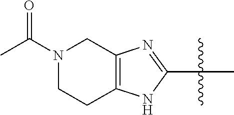 [SMILES: *c1nc2c([nH]1)CCN(C(C)=O)C2] | Me | H, H | S | 0 | — | 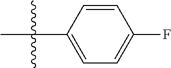 [SMILES: *c1ccc(F)cc1] |
| 127 | 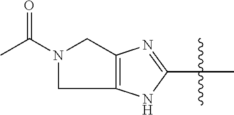 [SMILES: *c1nc2c([nH]1)CN(C(C)=O)C2] | H | H, H | O | 0 | — | 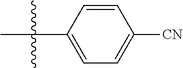 [SMILES: *c1ccc(C#N)cc1] |
| 128 | 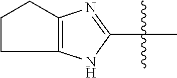 [SMILES: *c1nc2c([nH]1)CCC2] | Me | Me, Me | O | 0 | — | 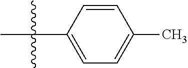 [SMILES: *c1ccc(C)cc1] |
| 129 | 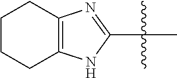 [SMILES: *c1nc2c([nH]1)CCCC2] | Ph | Me, Me | S | 0 | — | 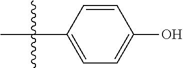 [SMILES: *c1ccc(O)cc1] |
| 130 | 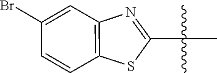 [SMILES: *c1nc2cc(Br)ccc2s1] | 4-pyridinyl | H, H | NMe | 1 | H, H | 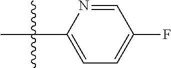 [SMILES: *c1ccc(F)cn1] |
| 131 | 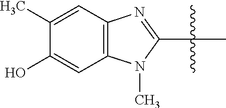 [SMILES: *c1nc2cc(C)c(O)cc2n1C] | Me | H, H | CH₂ | 1 | H, Me | 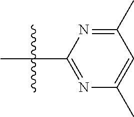 [SMILES: *c1nc(C)cc(C)n1] |
| 132 | 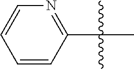 [SMILES: *c1ccccn1] | H | H, H | S | 0 | — | 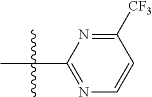 [SMILES: *c1nccc(C(F)(F)F)n1] |
| 133 | 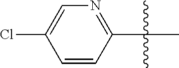 [SMILES: *c1ccc(Cl)cn1] | H | H, H | S | 1 | Me, Me | 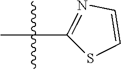 [SMILES: *c1nccs1] |
| 134 | 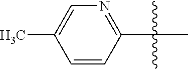 [SMILES: *c1ccc(C)cn1] | Me | H, H | S | 0 | — | 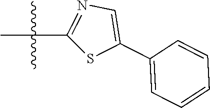 [SMILES: *c1ncc(-c2ccccc2)s1] |
| 135 | 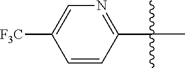 [SMILES: *c1ccc(C(F)(F)F)cn1] | H | H, H | S | 0 | — | 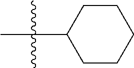 [SMILES: *C1CCCCC1] |
| 136 | 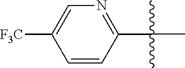 [SMILES: *c1ccc(C(F)(F)F)cn1] | H | H, H | NH | 0 | — | —SO₂Me |

### Abbreviations

ACN Acetonitrile
EA Ethyl acetate
DMF Dimethyl formamide
PE Petroleum ether
DCM Dichloromethane
THF Tetrahydrofuran
HOBT 1-Hydroxybenzotriazole
EDCI 1-Ethyl-3-(3-dimethylaminopropyl)carbodiimide
HBTU 2-(1H-Benzotriazole-1-yl)-1,1,3,3-tetramethyluronium hexafluorophosphate
HATU N-[(dimethylamino)(3H-1,2,3-triazolelo(4,4-b)pyridin-3-yloxy)methylene]-N-methylmethaneaminium hexafluorophosphate
PyBOP 1H-Benzotriazol-1-yloxytripyrrolidinophosphoniumhexafluorophosphate
BOPCl Bis(2-oxo-3-oxazolidinyl)phosphinic chloride
BOP Benzotriazol-1-yloxytris(diethylamino)phosphonium hexafluorophospahte
TEA Triethylamine
DIPEA Diisopropylethylamine
DMAP 4-Dimethylaminopyridine
PCC Pyridinium chlorochromate
PDC Pyridinium dichromate
NBS N-bromosuccinimide
NCS N-chlorosuccinimide
NIS N-iodosuccinimide
9-BBN 9-Borabicyclo[3.3.1]nonane
TsOH p-Toluenesulfonic acid
TFA Trifluoroacetamide
CDI Carbonyldiimidazole

### Methods of Preparation

Following are general synthetic schemes for manufacturing compounds of the present invention. These schemes are illustrative and are not meant to limit the possible techniques one skilled in the art may use to manufacture the compounds disclosed herein. Different methods will be evident to those skilled in the art. Additionally, the various steps in the synthesis may be performed in an alternate sequence or order to give the desired compound(s). All documents cited herein are incorporated herein by reference in their entirety. For example, the following reactions are illustrations but not limitations of the preparation of some of the starting materials and compounds disclosed herein.

Scheme 1 below describe a synthetic route which may be used for the synthesis of compounds of the present invention, e.g., compounds having a structure of Formula Ia. Various modifications to these methods may be envisioned by those skilled in the art to achieve similar results to that of the inventors given below. In the embodiments below, the synthetic route is described using a compound having the structure of Formula Ia as an example. The general synthetic route described in Scheme 1 and examples described in the Example section below illustrate methods used for the preparation of the compounds described herein.

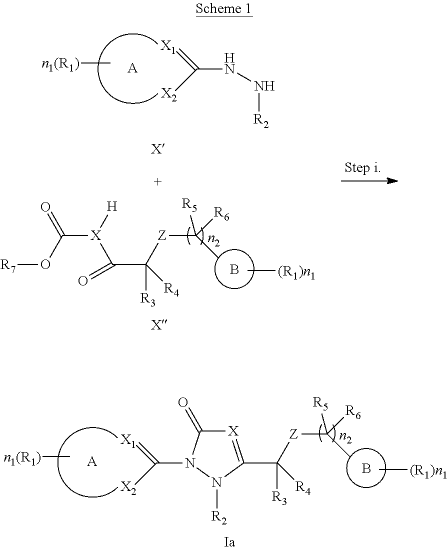

As shown in Scheme 1, a compound of Formula Ia can be prepared from a coupling/condensation reaction of a compound of Formula X′ and a compound of Formula X″. R₇ is H, alkyl, alkenyl, alkynyl, cycloalkyl, cycloalkenyl, cycloalkynyl, heterocycle, aryl, heteroaryl, alkyl-aryl, or alkyl-heteroaryl. The other substituents are as defined herein.

A compound of Formula X′ and a compound of Formula X″ can be prepared by any method known in the art. In Step i shown in Scheme 1, a compound of Formula X′ and a compound of Formula X″ can be mixed in a suitable solvent. Suitable solvents include, but are not limited to, acetonitrile, methanol, ethanol, dichloromethane, DMF, or toluene. The reaction may be conducted under inert atmosphere, e.g., under nitrogen or argon, or the reaction may be carried out in a sealed tube. The reaction mixture may be heated in a microwave or heated to an elevated temperature. Suitable elevated temperatures include, but are not limited to, 40, 50, 60, 80, 90, 100, 110, 120° C. or higher or the refluxing/boiling temperature of the solvent used. In some embodiments, an acid may be used to facilitate the reaction. Suitable acids include, but are not limited to, any organic or inorganic acid such as HCl, HBr, HI, H₂SO₄, or HAc. The reaction may be worked up by removing the solvent or partitioning of the organic solvent phase with one or more aqueous phases each optionally containing NaCl, NaHCO₃, or NH₄Cl. The solvent in the organic phase can be removed by reduced vacuum evaporation and the resulting residue may be purified using a silica gel column or HPLC.

### Pharmaceutical Compositions

This invention also provides a pharmaceutical composition comprising at least one of the compounds as described herein or a pharmaceutically acceptable salt or solvate thereof, and a pharmaceutically acceptable carrier.

In yet another aspect, the present invention provides a pharmaceutical composition comprising at least one compound selected from the group consisting of compounds of Formula Ia or Ib as described herein and a pharmaceutically-acceptable carrier or diluent.

In certain embodiments, the composition is in the form of a hydrate, solvate or pharmaceutically acceptable salt. The composition can be administered to the subject by any suitable route of administration, including, without limitation, oral and parenteral.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as butylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. The term “carrier” denotes an organic or inorganic ingredient, natural or synthetic, with which the active ingredient is combined to facilitate the application. The components of the pharmaceutical compositions also are capable of being comingled with the compounds of the present invention, and with each other, in a manner such that there is no interaction which would substantially impair the desired pharmaceutical efficiency.

As set out above, certain embodiments of the present pharmaceutical agents may be provided in the form of pharmaceutically acceptable salts. The term “pharmaceutically-acceptable salt”, in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al., (1977) “Pharmaceutical Salts”, J. Pharm. Sci. 66:1-19.)

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, butionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra.)

Wetting agents, emulsifiers and lubricants, such as sodium lauryl sulfate, magnesium stearate, and polyethylene oxide-polybutylene oxide copolymer as well as coloring agents, release agents, coating agents, sweetening, flavoring and perfuming agents, preservatives and antioxidants can also be present in the compositions.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the compound which produces a therapeutic effect. Generally, out of 100%, this amount will range from about 1% to about 99% of active ingredient, preferably from about 5% to about 70%, most preferably from about 10% to about 30%.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polybutylene oxide copolymer; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxybutylmethyl cellulose), lubricant, inert diluent, preservative, disintegrant (for example, sodium starch glycolate or cross-linked sodium carboxymethyl cellulose), surface-active or dispersing agent. Molded tablets, may be, made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxybutylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isobutyl alcohol, ethyl carbonate, ethyl acetate, benzyl alcohol, benzyl benzoate, butylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Additionally, cyclodextrins, e.g., hydroxybutyl-β-cyclodextrin, may be used to solubilize compounds.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and butane.

Transdermal patches have the added advantage of providing controlled delivery of a compound of the present invention to the body. Such dosage forms can be made by dissolving, or dispersing the pharmaceutical agents in the proper medium. Absorption enhancers can also be used to increase the flux of the pharmaceutical agents of the invention across the skin. The rate of such flux can be controlled, by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle. One strategy for depot injections includes the use of polyethylene oxide-polypropylene oxide copolymers wherein the vehicle is fluid at room temperature and solidifies at body temperature.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly (orthoesters) and poly (anhydrides). Depot injectable formulations are also prepared by entrapping the drug in liposomes or microemulsions, which are compatible with body tissue.

When the compounds of the present invention are administered as pharmaceuticals, to humans and animals, they can be given per se or as a pharmaceutical composition containing, for example, 0.1% to 99.5% (more preferably, 0.5% to 90%) of active ingredient in combination with a pharmaceutically acceptable carrier.

The compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, the compound of the present invention may be administered concurrently with another anticancer agents).

The compounds of the invention may be administered intravenously, intramuscularly, intraperitoneally, subcutaneously, topically, orally, or by other acceptable means. The compounds may be used to treat arthritic conditions in mammals (e.g., humans, livestock, and domestic animals), race horses, birds, lizards, and any other organism, which can tolerate the compounds.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### Administration to a Subject

In yet another aspect, the present invention provides a method for treating cancer in a mammalian species in need thereof, the method comprising administering to the mammalian species a therapeutically effective amount of at least one compound selected from the group consisting of compounds of Formula Ia or Ib.

In some embodiments, the mammalian species is human. In some embodiments, the cancer is selected from the group consisting of biliary tract cancer, brain cancer, breast cancer, cervical cancer, choriocarcinoma, colon cancer, endometrial cancer, esophageal cancer, gastric (stomach) cancer, intraepithelial neoplasms, leukemias, lymphomas, liver cancer, lung cancer, melanoma, neuroblastomas, oral cancer, ovarian cancer, pancreatic cancer, prostate cancer, rectal cancer, renal (kidney) cancer, sarcomas, skin cancer, testicular cancer, and thyroid cancer.

In yet another aspect, a method of inhibiting Ras protein in a mammalian species in need thereof is described, including administering to the mammalian species a therapeutically effective amount of at least one compound selected from the group consisting of compounds of Formula Ia and Ib.

Some aspects of the invention involve administering an effective amount of a composition to a subject to achieve a specific outcome. The small molecule compositions useful according to the methods of the present invention thus can be formulated in any manner suitable for pharmaceutical use.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the compound can be administered to a subject by any mode allowing the compound to be taken up by the appropriate target cells. “Administering” the pharmaceutical composition of the present invention can be accomplished by any means known to the skilled artisan. Specific routes of administration include, but are not limited to, oral, transdermal (e.g., via a patch), parenteral injection (subcutaneous, intradermal, intramuscular, intravenous, intraperitoneal, intrathecal, etc.), or mucosal (intranasal, intratracheal, inhalation, intrarectal, intravaginal, etc.). An injection can be in a bolus or a continuous infusion.

For example the pharmaceutical compositions according to the invention are often administered by intravenous, intramuscular, or other parenteral means. They can also be administered by intranasal application, inhalation, topically, orally, or as implants, and even rectal or vaginal use is possible. Suitable liquid or solid pharmaceutical preparation forms are, for example, aqueous or saline solutions for injection or inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of present methods for drug delivery, see Langer R (1990) Science 249:1527-33, which is incorporated herein by reference.

The concentration of compounds included in compositions used in the methods of the invention can range from about 1 nM to about 100 μM. Effective doses are believed to range from about 10 picomole/kg to about 100 micromole/kg.

The pharmaceutical compositions are preferably prepared and administered in dose units. Liquid dose units are vials or ampoules for injection or other parenteral administration. Solid dose units are tablets, capsules, powders, and suppositories. For treatment of a patient, depending on activity of the compound, manner of administration, purpose of the administration (i.e., prophylactic or therapeutic), nature and severity of the disorder, age and body weight of the patient, different doses may be necessary. The administration of a given dose can be carried out both by single administration in the form of an individual dose unit or else several smaller dose units. Repeated and multiple administration of doses at specific intervals of days, weeks, or months apart are also contemplated by the invention.

The compositions can be administered per se (neat) or in the form of a pharmaceutically acceptable salt. When used in medicine the salts should be pharmaceutically acceptable, but non-pharmaceutically acceptable salts can conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Compositions suitable for parenteral administration conveniently include sterile aqueous preparations, which can be isotonic with the blood of the recipient. Among the acceptable vehicles and solvents are water, Ringer's solution, phosphate buffered saline, and isotonic sodium chloride solution. In addition, sterile, fixed oils are conventionally employed as a solvent or suspending medium. For this purpose, any bland fixed mineral or non-mineral oil may be employed including synthetic mono- or diglycerides. In addition, fatty acids such as oleic acid find use in the preparation of injectables. Carrier formulations suitable for subcutaneous, intramuscular, intraperitoneal, intravenous, etc. administrations can be found in Remington's Pharmaceutical Sciences, Mack Publishing Company, Easton, Pa.

The compounds useful in the invention can be delivered in mixtures of more than two such compounds. A mixture can further include one or more adjuvants in addition to the combination of compounds.

A variety of administration routes is available. The particular mode selected will depend, of course, upon the particular compound selected, the age and general health status of the subject, the particular condition being treated, and the dosage required for therapeutic efficacy. The methods of this invention, generally speaking, can be practiced using any mode of administration that is medically acceptable, meaning any mode that produces effective levels of response without causing clinically unacceptable adverse effects. Preferred modes of administration are discussed above.

The compositions can conveniently be presented in unit dosage form and can be prepared by any of the methods well known in the art of pharmacy. All methods include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product.

Other delivery systems can include time-release, delayed release or sustained release delivery systems. Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

### Assays for Effectiveness of Ras Protein Inhibition

In some embodiments, the IC₅₀ assay measures the reduction effect of a given compound on the guanine nucleotide-echange rate of Ras protein in the presence of guanine nucleotide exchange protein SOS1, which is a Ras activator. In the assay GDP-loaded Ras was first prepared in a buffer containing the compound at certain concentration and fluorescent guanine nucleotide (mant-GDP (used in Icagene assay) or mant-GTP (used in other IC₅₀ assays). The guanine nucleotide exchange was initiated by the addition of the SOS1 and its rate was reflected in the rate of fluorescence change as a function of time, which was monitored by a reader. The experiment was repeated at different compound concentrations and the IC₅₀ of the compound was defined as the compound concentration at which half of its maximal reduction effect on the guanine nucleotide exchange rate is achieved. In other embodiments, other Ras activator proteins may be used. Non-limiting examples of other Ras activator proteins include RasGRP (Ras guanyl-releasing protein).

### Equivalents

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification and guidance which can be adapted to the practice of this invention in its various embodiments and equivalents thereof.

### EXAMPLES

### Example 1. Synthesis of methyl 3-oxo-4-(phenylthio)butanoate

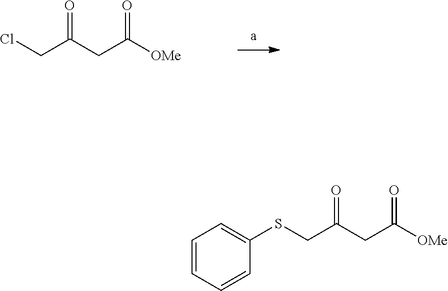 [SMILES: COC(=O)CC(=O)CCl ; COC(=O)CC(=O)CSc1ccccc1]

Step a: To a solution of thiophenol (435 μL, 4.236 mmol) in EtOH (8.47 mL) were added NaOAc (521.3 mg, 6.354 mmol) and methyl-4-chloroacetoacetate (498.3 μL, 4.236 mmol). The mixture was stirred at reflux for 1 h and EtOH was then evaporated. The residue was diluted with H₂O (30 mL) and extracted with EtOAc (3×30 mL). The combined organic phases were dried over MgSO₄, filtered, and the volatiles were removed under reduced pressure to give methyl 3-oxo-4-(phenylthio)butanoate (950 mg, 4.236 mmol) as a pale brown oil. ¹H NMR (500 MHz, CDCl₃) δ 7.36-7.27 (m, 4H), 7.25-7.21 (m, 1H), 3.80 (s, 2H), 3.71 (s, 3H), 3.65 (s, 2H). MS (ES⁺) m/z 225.05 (M+1).

### Example 2. Synthesis of methyl 2-methyl-3-oxo-4-(phenylthio)butanoate

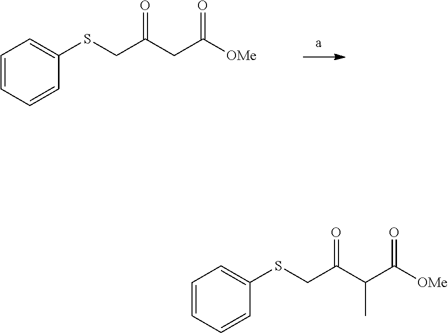 [SMILES: COC(=O)CC(=O)CSc1ccccc1 ; COC(=O)C(C)C(=O)CSc1ccccc1]

Step a: To a solution of methyl 3-oxo-4-(phenylthio)butanoate (50 mg, 0.223 mmol) in THF (0.22 mL) were added t-BuOK (27.5 mg, 0.245 mmol) and MeI (15.3 μL, 0.245 mmol). The mixture was stirred at 60° C. for 16 h. Water (10 mL) was added to the solution and the organic layers were extracted with EtOAc (3×20 mL). The combined organic phases were washed with brine, dried over MgSO₄, filtered, and the volatiles were removed under reduced pressure to give methyl 2-methyl-3-oxo-4-(phenylthio)butanoate as a yellow residue. MS (ES⁺) m/z 239.1 (M+1).

### Example 3. Synthesis of 2-hydrazinyl-1H-benzo[d]imidazole

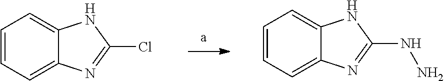 [SMILES: Clc1nc2ccccc2[nH]1 ; NNc1nc2ccccc2[nH]1]

Step a: A mixture of chlorobenzimidazole (1 g, 6.554 mmol) in hydrazine hydrate (10.2 mL) was stirred overnight at 140° C. The precipitate was filtered and washed with cold water to give 2-hydrazinyl-1H-benzo[d]imidazole (900 mg, 6.07 mmol) as a grey solid. MS (ES⁺) m/z 149.0 (M+1).

### Example 4. Synthesis of 2-hydrazinyl-5-methylpyridine

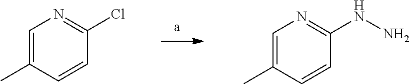

Step a: A mixture of 2-chloro-5-methylpyridine (100 mg, 0.784 mmol) in hydrazine hydrate (1.22 mL) was stirred overnight at 140° C. Water (10 mL) was added to the solution followed by NaOH and the organic layers were extracted with dichloromethane (3×20 mL). The combined organic phases were dried over MgSO₄, filtered, and the volatiles were removed under reduced pressure to give 2-hydrazinyl-5-methylpyridine (55 mg, 0.446 mmol) as a yellow residue. MS (ES⁺) m/z 123.97 (M+1).

### Example 5. Synthesis of 2-hydrazineyl-1H-benzo[d]imidazole-5-carboxylic acid

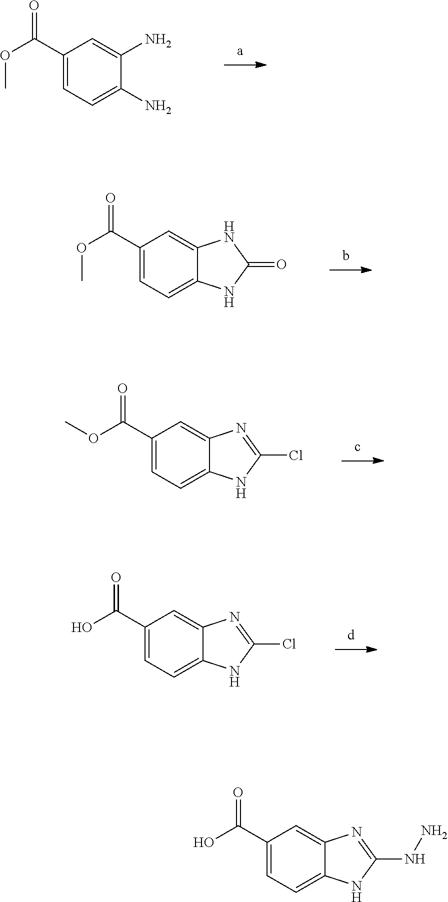

Step a: To a solution of methyl 3,4-diaminobenzoate (2 g, 12.0 mmol) in DMF (10 mL) was added 1-[(1H-imidazol-1-yl) carbonyl]-1H-imidazole (1.95 g, 12.0 mmol) at room temperature. The mixture was stirred at room temperature under nitrogen atmosphere for 3 h at which time the resulting suspension was filtered. The filter cake was washed with EtOH (20 mL) and dried under vacuum to afford methyl 2-oxo-2,3-dihydro-1H-1,3-benzodiazole-5-carboxylate (1.5 g, 7.8 mmol, yield 65%) as a brown solid: LCMS (ESI) calc'd C₉H₈N₂O₃ for [M+H]⁺: 193, found 193; ¹H NMR (300 MHz, DMSO-d₆) δ ppm 11.06 (s, 1H), 10.89 (s, 1H), 7.63 (d, J=8.2, 1H), 7.47 (s, 1H), 7.02 (d, J=8.2 Hz, 1H), 3.82 (s, 3H).

Step b: To a round-bottom flask were changed methyl 2-oxo-2,3-dihydro-1H-1,3-benzodiazole-5-carboxylate (1.5 g, 7.8 mmol) and POCl₃ (10 mL, 107 mmol). The reaction mixture was heated at 100° C. under nitrogen atmosphere for 16 h at which time the reaction was cooled and quenched by the addition of water (100 mL). The resulting heterogeneous solution was filtered. The filter cake was washed with water (20 mL) and dried under vacuum to afford methyl 2-chloro-1H-1,3-benzodiazole-6-carboxylate (1.2 g, 5.7 mmol, yield 73%) as a brown solid: LCMS (ESI) calc'd C₉H₇ClN₂O₂ for [M+H]⁺: 211, 213 (3:1) found 211, 213 (3:1); ¹H NMR (300 MHz, DMSO-d₆) δ ppm 8.15-8.06 (m, 1H), 7.86 (dd, J=8.5, 1.6 Hz, 1H), 7.61 (dd, J=8.5, 0.7 Hz, 1H), 3.87 (s, 3H).

Step c: To a solution of methyl 2-chloro-1H-1,3-benzodiazole-6-carboxylate (1.2 g, 5.7 mmol) in EtOH (50 mL) was added an aqueous solution of NaOH (w/w 20%, 5 mL) at room temperature. The reaction was then heated at 50° C. for 16 h at which time it was cooled and quenched by the addition of 6 N HCl (5 mL). The resulting mixture was concentrated under vacuum. The residue was washed with water (10 mL) and dried under vacuum to afford 2-chloro-1H-1,3-benzodiazole-6-carboxylic acid (1 g, crude) as a brown solid: LCMS (ESI) calc'd C₈H₅ClN₂O₂ for [M+H]⁺: 197, 199 (3:1) found 197, 199 (3:1); ¹H NMR (300 MHz, DMSO-d₆) δ ppm 8.09 (s, 1H), 7.86 (d, J=8.4 Hz, 1H), 7.59 (d, J=8.4 Hz, 1H).

Step d: To a suspension of 2-chloro-1H-1,3-benzodiazole-6-carboxylic acid (0.30 g, 1.5 mmol) in NMP (20 mL) was added hydrazine hydrate (98%) (3 mL, 61.7 mmol) at room temperature. The reaction mixture was heated at 140° C. under nitrogen atmosphere for 3 h at which time it was concentrated under vacuum. The resulted residue was purified by prep-HPLC using the following conditions: Column: XBridge Prep Amide OBD Column 19×150 mm, 5 m, 13 nm; mobile Phase A: water (20 mmol/L NH₄HCO₃), mobile Phase B: CH₃CN; flow rate: 20 mL/min; gradient: 90% A to 40% A in 12 min; detector: UV 254 nm; retention time: 7.4 to 8.6 min. The eluents containing the desired product were concentrated to afford 2-hydrazinyl-1H-1,3-benzodiazole-6-carboxylic acid (0.1 g, 0.5 mmol, yield 34%) as a purple solid: LCMS (ESI) calc'd C₈H₈N₄O₂ for [M+H]⁺: 193 found 193; ¹H NMR (300 MHz, DMSO-d₆) δ ppm 7.71 (d, J=1.6 Hz, 1H), 7.56 (dd, J=8.2, 1.8 Hz, 1H), 7.11 (dd, J=8.2, 2.8 Hz, 1H).

### Example 6. Synthesis of Compound 137: 2-(4-methyl-3-(((5-methyl-1,3,4-thiadiazol-2-yl)thio)methyl)-5-oxo-2,5-dihydro-1H-pyrazol-1-yl)-1H-benzo[d]imidazole-6-carboxylic acid trifluoacetate salt

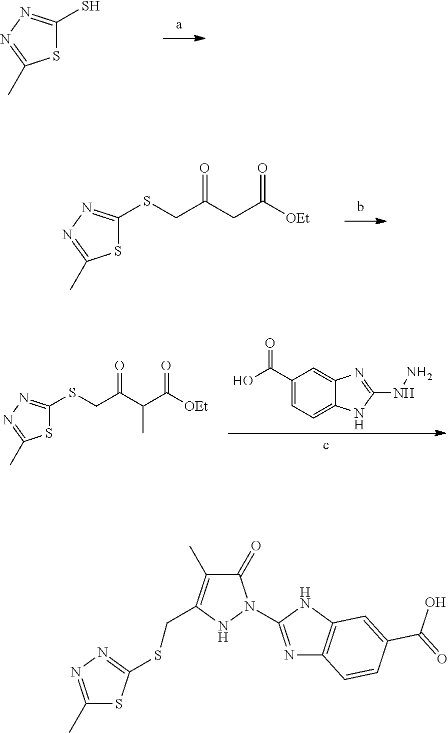

Step a: To a solution of 5-methyl-1,3,4-thiadiazole-2-thiol (10.0 g, 75.6 mmol) and ethyl 4-chloro-3-oxobutanoate (14.9 g, 90.5 mmol) in DMF (100 mL) was added K₂CO₃ (15.7 g, 113.6 mmol) at room temperature. The reaction mixture was heated at 60° C. under nitrogen atmosphere for 2 h at which time it was cooled and diluted with CH₂Cl₂ (500 mL) and washed with brine (250 mL×3). The separated organic phase was dried over anhydrous Na₂SO₄, filtered and concentrated under vacuum. The residue was purified by silica gel column chromatography, eluting with p-ether and ethyl acetate (10:1 to 1:1) to afford ethyl 4-[(5-methyl-1,3,4-thiadiazol-2-yl)sulfanyl]-3-oxobutanoate (16 g, 61 mmol, yield 81%) as an off-white solid: LCMS (ESI) calc'd C₉H₁₂N₂O₃S₂ for [M+H]⁺: 261, found 261; ¹H NMR (300 MHz, DMSO-d₆) δ ppm 4.47 (s, 2H), 4.10 (q, J=7.1 Hz, 2H), 3.79 (s, 2H), 2.67 (s, 3H), 1.19 (t, J=7.1 Hz, 3H).

Step b: To a solution of ethyl 4-[(5-methyl-1,3,4-thiadiazol-2-yl)sulfanyl]-3-oxobutanoate (1.0 g, 3.8 mmol) in THF (20 mL) was added in one portion t-BuOK (0.47 g, 4.2 mmol) at room temperature. This mixture was stirred at room temperature under nitrogen atmosphere for 20 min. Methyl iodide (0.60 g, 4.23 mmol) was added to the solution and the reaction stirred at room temperature under nitrogen atmosphere for additional 3 h. The reaction mixture was diluted with CH₂Cl₂ (400 mL) and washed with water (200 mL×3). The separated organic phase was dried over anhydrous Na₂SO₄, filtered and concentrated under vacuum. The residue was purified by silica gel column chromatography, eluting with p-ether and ethyl acetate (10:1 to 1:1) to afford ethyl 2-methyl-4-[(5-methyl-1,3,4-thiadiazol-2-yl)sulfanyl]-3-oxobutanoate (0.67 g, 2.4 mmol, yield 67%) as a light yellow oil: LCMS (ESI) calc'd C₁₀H₁₄N₂O₃S₂ for [M+H]⁺: 275, found 275; ¹H NMR (300 MHz, DMSO-d₆) δ ppm 4.46 (s, 2H), 4.12 (q, J=7.1 Hz, 2H), 4.00 (q, J=7.1 Hz, 1H), 2.67 (s, 3H), 1.25 (d, J=7.1 Hz, 3H), 1.19 (t, J=7.1 Hz, 3H).

Step c: To a solution of 2-hydrazinyl-1H-1,3-benzodiazole-6-carboxylic acid (70 mg, 0.4 mmol) in CH₃CN (10 mL) were added ethyl 2-methyl-3-[(5-methyl-1,3,4-thiadiazol-2-yl)sulfanyl]-3-oxopropanoate (0.10 g, 0.4 mmol) and acetic acid (1 mL). The mixture was heated at 80° C. under nitrogen atmosphere for 16 h at which time it was cooled and concentrated under vacuum. The residue was purified by prep-HPLC using the following conditions: column: XBridge C18 OBD Prep Column 100 Å, 10 m, 19 mm×250 mm; mobile Phase A: water (0.05% TFA), mobile Phase B: ACN; flow rate: 20 mL/min; gradient: 25% B to 60% B in 6.5 min; detector: UV 254/210 nm; retention time: 5.55 min. The fractions containing the desired product were concentrated to afford Compound 137, 2-(4-methyl-3-[[(5-methyl-1,3,4-thiadiazol-2-yl)sulfanyl]methyl]-5-oxo-2,5-dihydro-1H-pyrazol-1-yl)-1H-1,3-benzodiazole-5-carboxylic acid as a trifluoroacetate salt (12.4 mg, 0.025 mmol, yield 7%) as an off-white solid: LCMS (ESI) calc'd C₁₆H₁₄N₆O₃S₂ for [M+H]⁺: 403, found 403; ¹H NMR (300 MHz, DMSO-d₆) δ ppm 8.12 (s, 1H), 7.83 (dd, J=8.5, 1.6 Hz, 1H), 7.60 (d, J=8.4 Hz, 1H), 4.50 (s, 2H), 2.69 (s, 3H), 1.90 (s, 3H). ¹⁹F NMR (300 MHz, DMSO-d₆) δ ppm −74.24.

### Example 7. Synthesis of Compound 138, 6-(3-[[(4,6-Dimethylpyrimidin-2-yl)sulfanyl]methyl]-4-methyl-5-oxo-2,5-dihydro-1H-pyrazol-1-yl)pyridine-3-carboxylic acid 2,2,2-trifluoroacetate

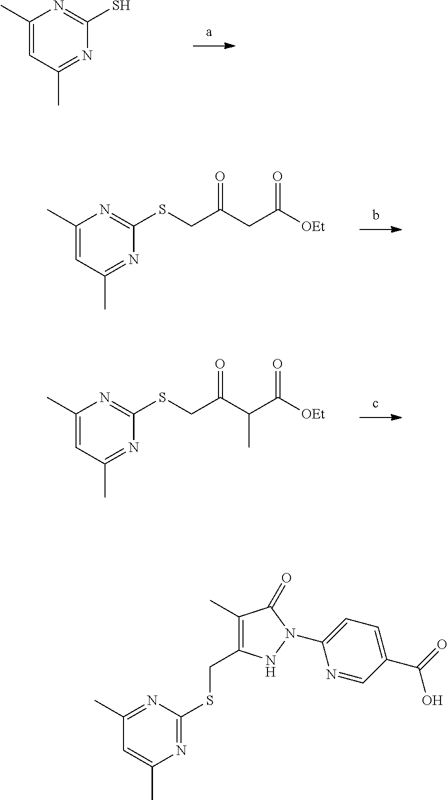

Step a: To a solution of 4,6-dimethylpyrimidine-2-thiol (12 g, 85.6 mmol) and ethyl 4-chloro-3-oxobutanoate (16.9 g, 102.8 mmol) in DMF (100 mL), K₂CO₃ (17.8 g, 128.4 mmol) was added in five portions at room temperature. The mixture was vigorously stirred while being heated at 60° C. under nitrogen atmosphere for 2 h. The reaction was cooled and diluted with CH₂Cl₂ (500 mL) and washed with brine (250 mL×3). The organic phase was separated, dried over anhydrous Na₂SO₄ and filtered. The filtrate was concentrated under vacuum. The residue was purified by silica gel column chromatography, eluting with p-ether and ethyl acetate (10:1 to 1:1) to afford ethyl 4-[(4,6-dimethylpyrimidin-2-yl)sulfanyl]-3-oxobutanoate (10.8 g, 40 mmol, yield 47%) as a light yellow oil: LCMS (ESI) calc'd for C₁₂H₁₆N₂O₃S [M+H]⁺: 269, found 269; ¹H NMR (300 MHz, DMSO-d₆) δ ppm 6.98 (s, 1H), 4.18-4.01 (m, 4H), 3.80 (s, 2H), 2.34 (s, 6H), 1.18 (t, J=7.1 Hz, 3H).

Step b: To a solution of ethyl 4-[(4,6-dimethylpyrimidin-2-yl)sulfanyl]-3-oxobutanoate (0.5 g, 1.86 mmol) in THF (10 mL), t-BuOK (0.23 g, 2.1 mmol) was added in one portion. The mixture was stirred at room temperature for 20 min at which time MeI (0.29 g, 2.1 mmol) was added. The reaction was then stirred at room temperature for additional 3 h. The reaction was then diluted with CH₂Cl₂ (200 mL) and washed with water (50 mL×3). The separated organic phase was dried over anhydrous Na₂SO₄ and filtered. The filtrate was concentrated under vacuum. The residue was purified by silica gel column chromatography, eluting with p-ether and ethyl acetate (10:1 to 1:1) to afford ethyl 4-[(4,6-dimethylpyrimidin-2-yl)sulfanyl]-2-methyl-3-oxobutanoate (0.37 g, 1.3 mmol, yield 70%) as a light yellow oil: LCMS (ESI) calc'd for C₁₃H₁₈N₂O₃S [M+H]⁺: 283, found 283; ¹H NMR (300 MHz, DMSO-d₆) δ ppm 6.97 (s, 1H), 4.29-3.96 (m, 5H), 2.33 (s, 6H), 1.27 (d, J=7.2 Hz, 3H), 1.18 (t, J=7.1, 3H).

Step c: To a solution of 6-hydrazinylpyridine-3-carboxylic acid (27.1 mg, 0.2 mmol) in CH₃CN (5 mL) were added ethyl 4-[(4,6-dimethylpyrimidin-2-yl)sulfanyl]-2-methyl-3-oxobutanoate (50 mg, 0.2 mmol) and acetic acid (0.1 mL). The mixture was heated at 80° C. under nitrogen atmosphere for 2 h at which time it was cooled and concentrated under reduced pressure. The residue was then purified by prep-HPLC using the following conditions: column: XBridge C18 OBD Prep Column 100 Å, 10 m, 19 mm×250 mm; mobile phase A: water (0.05% TFA); mobile phase B: CH₃CN; flow rate: 20 mL/min; gradient: 5% B to 30% B in 6.5 min; detector: UV 254/210 nm; retention time: 6.15 min. The fractions containing the desired product were concentrated under reduced pressure to afford Compound 138, 6-(3-[[(4,6-dimethylpyrimidin-2-yl) sulfanyl]methyl]-4-methyl-5-oxo-2,5-dihydro-1H-pyrazol-1-yl)pyridine-3-carboxylic acid (a trifluoroacetate salt; 14.7 mg, yield 17%) as an off-white solid: LCMS (ESI) calc'd for C₁₇H₁₇N₅O₃S [M+H]⁺: 372, found 372; ¹H NMR (300 MHz, DMSO-d₆) δ ppm 8.91 (s, 1H), 8.64-8.20 (m, 2H), 7.02 (s, 1H), 4.36 (s, 2H), 2.41 (s, 6H), 1.85 (s, 3H); ¹⁹F NMR (300 MHz, DMSO-d₆) δ ppm −73.55.

### Example 8. Synthesis of Compound 139: 2-(1H-benzo[d]imidazol-2-yl)-5-(((4-ethylthiazol-2-yl)thio)methyl)-1H-pyrazol-3(2H)-one

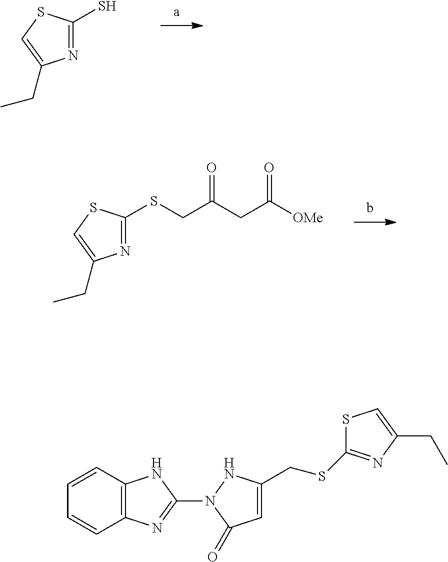

Step a: To a solution of 4-ethyl-2-thio-1,3-thiazole (50 mg, 0.344 mmol) in EtOH (0.69 mL) were added NaOAc (42.4 mg, 0.516 mmol) and methyl-4-chloroacetoacetate (40.5 μL, 0.344 mmol). The mixture was stirred at reflux for 2 h and EtOH was then evaporated. The residue was diluted with H₂O (10 mL) and extracted with EtOAc (3×10 mL). The combined organic phases were dried over MgSO₄, filtered, and the volatiles were removed under reduced pressure to give methyl 4-((4-ethylthiazol-2-yl)thio)-3-oxobutanoate (89 mg, 0.344 mmol) as a pale brown oil. MS (ES+) m/z 260.2 (M+1).

Step b: To a flask equipped with a magnetic stir bar methyl 4-((4-ethylthiazol-2-yl)thio)-3-oxobutanoate (70 mg, 0.270 mmol) and 2-hydrazinyl-1H-benzo[d]imidazole (40 mg, 0.270 mmol) were dissolved in acetonitrile (0.54 mL). The reaction was stirred at reflux for 2 h and then concentrated under reduced pressure. The crude was purified by reverse chromatography (10 to 80% gradient of MeOH/10 mM aqueous ammonium formate) to give Compound 139, 2-(1H-benzo[d]imidazol-2-yl)-5-(((4-ethylthiazol-2-yl)thio)methyl)-1H-pyrazol-3(2H)-one (32.5 mg, 0.090 mmol) as an off-white powder. ¹H NMR (500 MHz, DMSO) δ 7.47 (dd, J=5.9, 3.2 Hz, 2H), 7.21-7.12 (m, 3H), 5.24 (s, 1H), 4.26 (s, 2H), 2.62 (qd, J=7.5, 0.9 Hz, 2H), 1.14 (t, J=7.5 Hz, 3H). MS (ES⁺) m/z 358.3 (M+1).

### Example 9. Synthesis of Compound 140: 2-(5-methylpyridin-2-yl)-5-((phenylthio)methyl)-1H-pyrazol-3(2H)-one

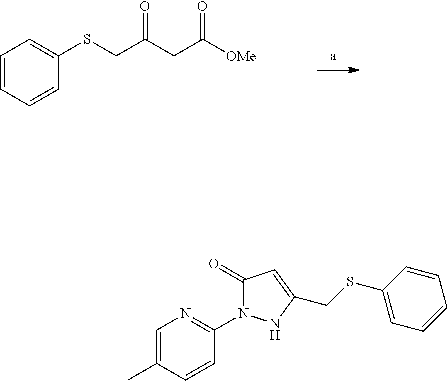 [SMILES: COC(=O)CC(=O)CSc1ccccc1 ; Cc1ccc(-n2[nH]c(CSc3ccccc3)cc2=O)nc1]

Step a: To a flask equipped with a magnetic stir bar methyl 3-oxo-4-(phenylthio)butanoate (91 mg, 0.406 mmol) and 2-hydrazinyl-5-methylpyridine (50 mg, 0.406 mmol) were dissolved in acetonitrile (0.5 mL). The reaction was stirred at reflux for 3 hours and then concentrated under reduced pressure. The crude was purified by flash chromatography (0 to 20% gradient of EtOAc/Hexane) to give Compound 140, 2-(5-methylpyridin-2-yl)-5-((phenylthio)methyl)-1H-pyrazol-3(2H)-one (8 mg, 0.027 mmol) as an off-white powder. ¹H NMR (500 MHz, DMSO) δ 8.26 (dd, J=1.5, 0.8 Hz, 1H), 7.84 (s, 1H), 7.62 (s, 1H), 7.42-7.37 (m, 2H), 7.31 (dd, J=9.9, 5.6 Hz, 2H), 7.19 (t, J=7.1 Hz, 1H), 5.53 (s, 1H), 4.11 (s, 2H), 2.32 (s, 3H). MS (ES+) m/z found 298.15 (M+1).

### Example 10. Synthesis of Compound 141: 2-(1H-benzo[d]imidazol-2-yl)-4-methyl-5-((phenylthio)methyl)-1H-pyrazol-3(2H)-one

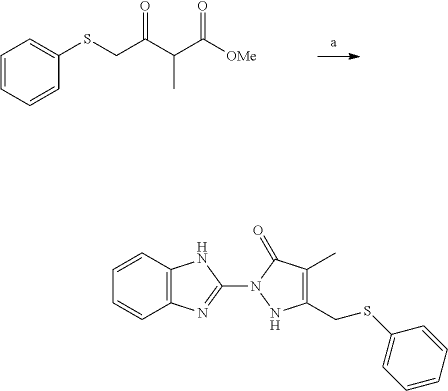 [SMILES: COC(=O)C(C)C(=O)CSc1ccccc1 ; Cc1c(CSc2ccccc2)[nH]n(-c2nc3ccccc3[nH]2)c1=O]

Step a: To a flask equipped with a magnetic stir bar, methyl 2-methyl-3-oxo-4-(phenylthio)butanoate (37 mg, 0.155 mmol) and 2-hydrazinyl-1H-benzo[d]imidazole (23 mg, 0.155 mmol) were dissolved in acetonitrile (0.2 mL). The reaction was stirred at reflux for 12 h and then concentrated under reduced pressure. The crude was purified by reverse chromatography (10 to 80% gradient of MeOH/10 mM aqueous ammonium formate) to give Compound 141, 2-(1H-benzo[d]imidazol-2-yl)-4-methyl-5-((phenylthio)methyl)-1H-pyrazol-3(2H)-one (32.2 mg, 0.009 mmol) as a yellow powder. ¹H NMR (500 MHz, DMSO) δ 7.53 (dd, J=5.8, 3.2 Hz, 2H), 7.48-7.41 (m, 2H), 7.38-7.30 (m, 2H), 7.24 (dd, J=10.5, 4.3 Hz, 1H), 7.21-7.16 (m, 2H), 4.15 (s, 2H), 1.79 (s, 3H). MS (ES+) m/z 337.2 (M+1).

### Example 11. Synthesis of Compound 142: 2-(1H-benzo[d]imidazol-2-yl)-5-((pyridin-2-ylthio)methyl)-1H-pyrazol-3(2H)-one

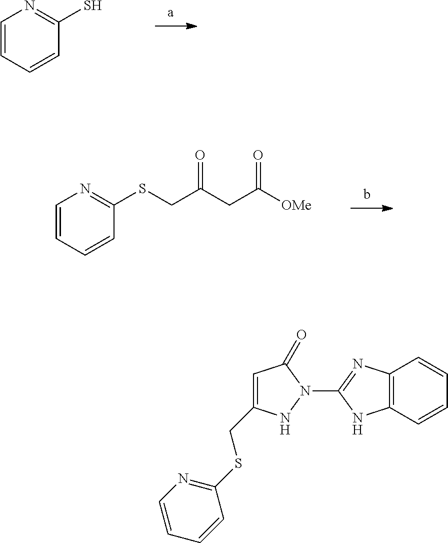 [SMILES: Sc1ccccn1 ; COC(=O)CC(=O)CSc1ccccn1 ; O=c1cc(CSc2ccccn2)[nH]n1-c1nc2ccccc2[nH]1]

Step a: To a solution of 2-mercaptopyridine (470 mg, 4.228 mmol) in EtOH (8.46 mL) were added NaOAc (520.3 mg, 6.342 mmol) and methyl-4-chloroacetoacetate (497.3 μL, 4.228 mmol). The mixture was stirred at reflux for 1 h and EtOH was then evaporated. The residue was diluted with H₂O (30 mL) and extracted with EtOAc (3×30 mL). The combined organic phases were dried over MgSO₄, filtered, and the volatiles were removed under reduced pressure to give methyl 3-oxo-4-(pyridin-2-ylthio)butanoate (952 mg, 4.228 mmol) as a pale brown oil. ¹H NMR (500 MHz, CDCl₃) δ 8.29 (ddd, J=5.0, 1.8, 1.0 Hz, 1H), 7.44 (ddd, J=8.0, 7.4, 1.8 Hz, 1H), 7.16 (ddd, J=4.6, 2.8, 1.8 Hz, 1H), 6.95 (dtd, J=6.0, 4.8, 1.0 Hz, 1H), 3.97 (s, 2H), 3.67 (s, 3H), 3.65 (s, 2H). MS (ES+) 226.04 (M+1).

Step b: To a flask equipped with a magnetic stir bar methyl 4-((2-chlorophenyl)thio)-3-oxobutanoate (100 mg, 0.444 mmol) and 2-hydrazinyl-1H-benzo[d]imidazole (65.8 mg, 0.444 mmol) were dissolved in acetonitrile (0.55 mL). The reaction was stirred at reflux for 2 h and then concentrated under reduced pressure. The crude was purified by reverse chromatography (5 to 70% gradient of ACN/10 mM aqueous ammonium formate) to give Compound 142, 2-(1H-benzo[d]imidazol-2-yl)-5-((pyridin-2-ylthio)methyl)-1H-pyrazol-3 (2H)-one (71 mg, 0.219 mmol) as a white powder. ¹H NMR (500 MHz, DMSO) δ 8.41 (ddd, J=4.9, 1.9, 0.9 Hz, 1H), 7.65-7.57 (m, 1H), 7.46 (dd, J=5.9, 3.2 Hz, 2H), 7.30 (d, J=8.1 Hz, 1H), 7.19-7.12 (m, 2H), 7.09 (ddd, J=7.4, 4.9, 1.0 Hz, 1H), 5.23 (s, 1H), 4.25 (s, 2H). MS (ES⁺) m/z found 324.21 (M+1).

### Example 12. Synthesis of Compound 143: 5-(((4,6-dimethylpyrimidin-2-yl)thio)methyl)-2-(pyridin-2-yl)-1,2-dihydro-3H-pyrazol-3-one

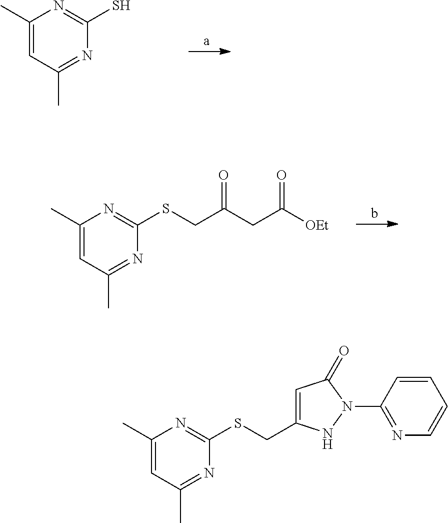

Step a: A solution of 2-mercapto-4,6-dimethylpyrimidine (1.2 g, 8.55 mmol) was cooled to 0° C. Ethyl 4-chloroacetoacetate (1.05 mL, 7.77 mmol) and trimethylamine (1.64 mL, 11.66 mmol) are added and the reaction is stirred at 0° C. for 30 minutes or until LC-MS shows full conversion. Reaction is then washed with saturated aqueous NaHCO₃, 0.25 N HCl, then brine. Solution is dried on magnesium sulfate, filtered and concentrated to dryness to give 2.1 g (100%) of desired intermediate, used as such without further purifications.

Step b: Thioether intermediate (523 mg, 1.95 mmol) and 2-hydrazinopyridine (212 mg, 1.95 mmol) were solubilized in absolute ethanol (5 mL) in a sealed tube. Reaction was heated to 50° C. for 5 h. Upon completion, reaction was concentrated to dryness. Crude material was purified via reversed-phase chromatography to yield 70 mg (11%) of desired Compound 143, 5-(((4,6-dimethylpyrimidin-2-yl)thio)methyl)-2-(pyridin-2-yl)-1,2-dihydro-3H-pyrazol-3-one. ¹H NMR (400 MHz, Methanol-d₄) δ ppm 8.38 (ddd, 1H, J₁=5.0 Hz, J₂=1.8 Hz, J₃=1.0 Hz), 8.05-8.03 (m, 1H), 7.94 (td, 1H, J₁=7.8 Hz, J₂=1.8 Hz), 7.26 (ddd, 1H, J₁=7.4 Hz, J₂=5.0 Hz, J₃=1.1 Hz), 6.95 (s, 1H), 4.34 (s, 2H), 2.44 (s, 6H). MS (ES⁺) m/z found 314.1 (M+1).

### Example 13. Synthesis of Compound 144: 2-(1H-benzo[d]imidazol-2-yl)-5-((phenylthio)methyl)-1,2-dihydro-3H-pyrazol-3-one

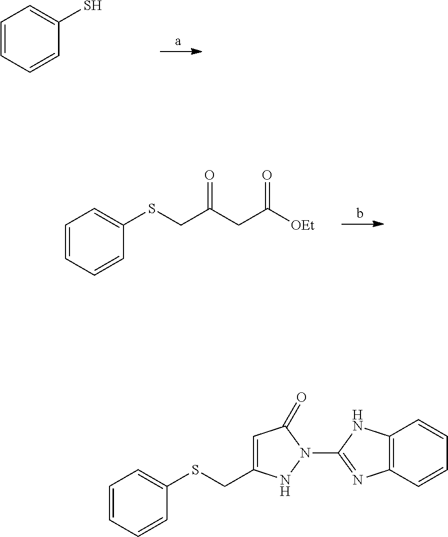 [SMILES: Sc1ccccc1 ; CCOC(=O)CC(=O)CSc1ccccc1 ; O=c1cc(CSc2ccccc2)[nH]n1-c1nc2ccccc2[nH]1]

Step a: A solution of thiophenol (500 mg, 4.54 mmol) was cooled to 0° C. Ethyl 4-chloroacetoacetate (0.61 mL, 4.54 mmol) and trimethylamine (0.944 mL, 6.81 mmol) are added and the reaction is stirred at 0° C. for 30 minutes or until LC-MS shows full conversion. Water is added. Mixture is extracted with ethyl acetate. Organic phases are then washed with saturated aqueous NaHCO₃, 0.25N HCl, then brine. Solution is dried on magnesium sulfate, filtered and concentrated to dryness. Compound is purified via reversed-phase chromatography to yield 224 mg (20%) of desired product.

Step b: Thioether intermediate (224 mg, 0.94 mmol) and (1H-Benzoimidazol-2-yl)-hydrazine (139 mg, 0.94 mmol) were solubilized in absolute ethanol (3 mL) in a sealed tube. Reaction was heated to 65° C. for 16 h. Upon completion, reaction was cooled to 0° C. White precipitate was collected by filtration, rinsed with ethanol and dried under vacuum to yield 121 mg (40%) of desired Compound 144, 2-(1H-benzo[d]imidazol-2-yl)-5-((phenylthio)methyl)-1,2-dihydro-3H-pyrazol-3-one. ¹H NMR (400 MHz, DMSO-d₆) δ ppm 7.52 (dd, 1H, J₁=6.0 Hz, J₂=3.2 Hz) 7.40 (d, 2H, J=7.8 Hz), 7.31 (t, 2H, J=7.6 Hz), 7.21-7.19 (m, 3H), 5.27 (s, 1H), 4.11 (s, 2H). MS (ES⁺) m/z found 323.1 (M+1).

### Example 14. Synthesis of Compound 145: 2-(1H-benzo[d]imidazol-2-yl)-5-(phenoxymethyl)-1,2-dihydro-3H-pyrazol-3-one

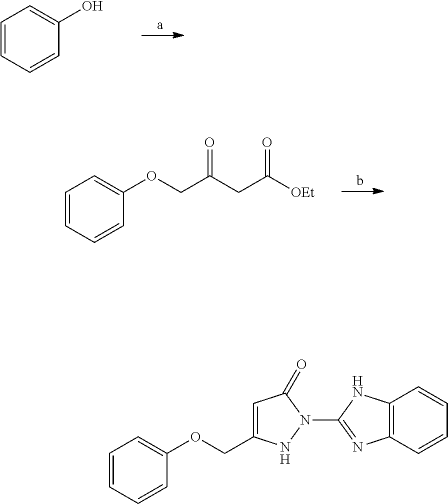 [SMILES: Oc1ccccc1 ; CCOC(=O)CC(=O)COc1ccccc1 ; O=c1cc(COc2ccccc2)[nH]n1-c1nc2ccccc2[nH]1]

Step a: A solution of phenol (376 mg, 4.0 mmol) in DMSO (0.8 mL) was added to a mixture of potassium hydroxide (449 mg, 8.0 mmol) in DMSO (8 mL) in a dry flask at r.t. The resulting mixture was stirred at r.t. for 30 minutes; ethyl 4-chloroacetoacetate (0.54 mL, 4.0 mmol) was then added to the mixture. The reaction mixture was stirred at r.t. overnight. Hydrochloric acid (4M) was added to acidify the reaction mixture. The resulting mixture was extracted with ethyl acetate three times. The combined organic phases were washed with water, then brine. The organic solution was dried over magnesium sulfate, filtered and concentrated under reduced pressure. The residue was purified by flash chromatography (EtOAc: hexanes, 0% to 15%) to give 527 mg, (59%) of desired product ethyl 3-oxo-4-phenoxybutanoate as colorless oil. ¹H NMR (400 MHz, CDCl₃) δ ppm 7.33-7.28 (m, 2H), 7.03-6.99 (m, 1H), 6.92-6.88 (m, 2H), 4.65 (s, 2H), 4.19 (q, 2H, J=7.1 Hz), 3.64 (s, 2H), 1.26 (t, 3H, J=7.2 Hz).

Step b: Ethyl 3-oxo-4-phenoxybutanoate (250 mg, 1.13 mmol) and (1H-Benzoimidazol-2-yl)-hydrazine (167 mg, 1.13 mmol) were solubilized in absolute ethanol (4.8 mL) in a sealed tube. The reaction mixture was stirred at r.t. for 1 h, and then heated to reflux overnight. Upon completion reaction was concentrated to dryness. Crude material was purified via reversed-phase chromatography to yield 42 mg (12%) of desired compound, Compound 145, 2-(1H-benzo[d]imidazol-2-yl)-5-(phenoxymethyl)-1,2-dihydro-3H-pyrazol-3-one. ¹H NMR (400 MHz, DMSO-d₆) δ ppm 8.03 (s, 1H), 7.92 (t, 1H, J=1.8 Hz), 7.69 (dt, 1H, J=7.7, 1.3 Hz), 7.57-7.53 (m, 3H), 7.44 (s, 1H), 7.40 (t, 1H, J=7.8 Hz), 7.24-7.20 (m, 2H), 5.29 (s, 1H), 4.18 (s, 2H). MS (ES⁺) m/z found 307.1 (M+1).

### Example 15. Compounds 146-187

Compounds 146-182 were prepared using procedures analogous to the synthetic procedures described for Compounds 137-145 above. The structures, chemical names, and characterization data for Compounds 146-182 are described below.

Compound 146: 2-(1H-benzo[d]imidazol-2-yl)-5-(((4,6-dimethylpyrimidin-2-yl)thio)methyl)-1,2-dihydro-3H-pyrazol-3-one, having the structure of

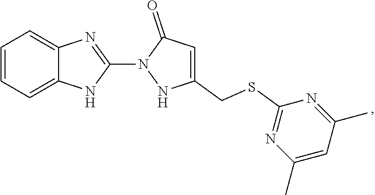 [SMILES: Cc1cc(C)nc(SCc2cc(=O)n(-c3nc4ccccc4[nH]3)[nH]2)n1]

was synthesized. MS (ES⁺) m/z found 353.1 (M+1).

Compound 147: 2-(1H-benzo[d]imidazol-2-yl)-5-(((5-methyl-1,3,4-thiadiazol-2-yl)thio)methyl)-1,2-dihydro-3H-pyrazol-3-one, having the structure of

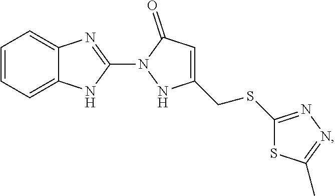 [SMILES: Cc1nnc(SCc2cc(=O)n(-c3nc4ccccc4[nH]3)[nH]2)s1]

was synthesized. MS (ES⁺) m/z found 345.1 (M+1).

Compound 148: 2-(1H-benzo[d]imidazol-2-yl)-5-(((4-fluorobenzyl)thio)methyl)-1,2-dihydro-3H-pyrazol-3-one, having the structure of

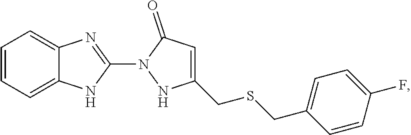 [SMILES: O=c1cc(CSCc2ccc(F)cc2)[nH]n1-c1nc2ccccc2[nH]1]

was synthesized. MS (ES⁺) m/z found 355.1 (M+1).

Compound 149: 2-(1H-benzo[d]imidazol-2-yl)-5-(((4-(trifluoromethyl)pyrimidin-2-yl)thio)methyl)-1,2-dihydro-3H-pyrazol-3-one, having the structure of

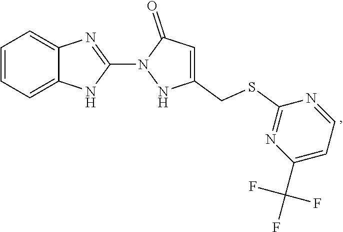 [SMILES: O=c1cc(CSc2nccc(C(F)(F)F)n2)[nH]n1-c1nc2ccccc2[nH]1]

was synthesized. MS (ES⁺) m/z found 393.1 (M+1).

Compound 150: 2-(1H-benzo[d]imidazol-2-yl)-5-(((3-methoxyphenyl)thio)methyl)-1,2-dihydro-3H-pyrazol-3-one, having the structure of

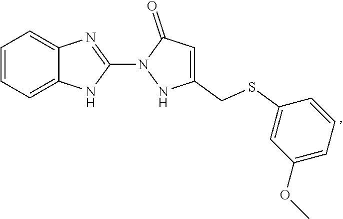 [SMILES: COc1cccc(SCc2cc(=O)n(-c3nc4ccccc4[nH]3)[nH]2)c1]

was synthesized. MS (ES⁺) m/z found 353.1 (M+1).

Compound 151: 2-(1H-benzo[d]imidazol-2-yl)-5-(((4-phenylthiazol-2-yl)thio)methyl)-1,2-dihydro-3H-pyrazol-3-one, having the structure of

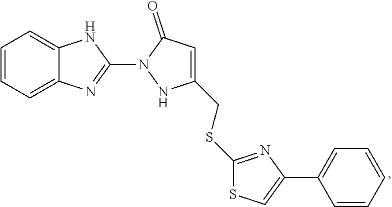 [SMILES: O=c1cc(CSc2nc(-c3ccccc3)cs2)[nH]n1-c1nc2ccccc2[nH]1]

was synthesized. MS (ES⁺) m/z found 406.1 (M+1).

Compound 152: 2-(1H-benzo[d]imidazol-2-yl)-5-(((4-methylthiazol-2-yl)thio)methyl)-1,2-dihydro-3H-pyrazol-3-one, having the structure of

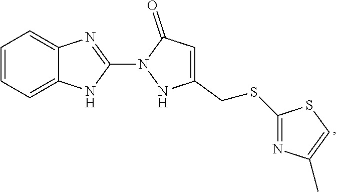 [SMILES: Cc1csc(SCc2cc(=O)n(-c3nc4ccccc4[nH]3)[nH]2)n1]

was synthesized. MS (ES⁺) m/z found 344.1 (M+1).

Compound 153: 2-(1H-benzo[d]imidazol-2-yl)-5-(((5-phenyl-1,3,4-oxadiazol-2-yl)thio)methyl)-1,2-dihydro-3H-pyrazol-3-one, having the structure of

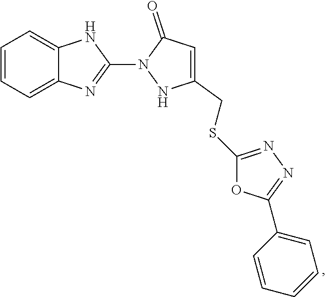 [SMILES: O=c1cc(CSc2nnc(-c3ccccc3)o2)[nH]n1-c1nc2ccccc2[nH]1]

was synthesized. MS (ES⁺) m/z found 391.3 (M+1).

Compound 154: 2-(1H-benzo[d]imidazol-2-yl)-5-(((5-chlorobenzo[d]thiazol-2-yl)thio)methyl)-1,2-dihydro-3H-pyrazol-3-one, having the structure of

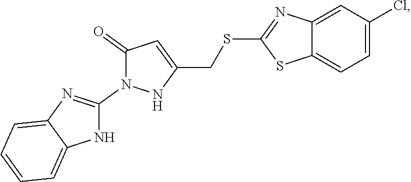 [SMILES: O=c1cc(CSc2nc3cc(Cl)ccc3s2)[nH]n1-c1nc2ccccc2[nH]1]

was synthesized. MS (ES⁺) m/z found 414.1 (M+1).

Compound 155: 2-(6-bromobenzo[d]thiazol-2-yl)-5-(((4,6-dimethylpyrimidin-2-yl)thio)methyl)-1,2-dihydro-3H-pyrazol-3-one, having the structure of

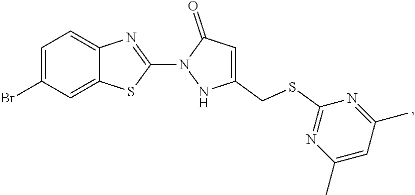 [SMILES: Cc1cc(C)nc(SCc2cc(=O)n(-c3nc4ccc(Br)cc4s3)[nH]2)n1]

was synthesized. MS (ES⁺) m/z found 448.0 and 450.0 (M+0; M+2).

Compound 156: 2-(1H-benzo[d]imidazol-2-yl)-5-((pyrimidin-2-ylthio)methyl)-1,2-dihydro-3H-pyrazol-3-one, having the structure of

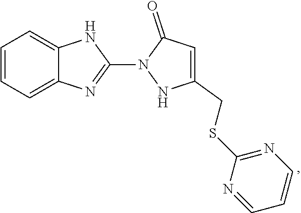 [SMILES: O=c1cc(CSc2ncccn2)[nH]n1-c1nc2ccccc2[nH]1]

was synthesized. MS (ES⁺) m/z found 325.1 (M+1).

Compound 157: 5-(((4,6-dimethylpyrimidin-2-yl)thio)methyl)-2-(5-(trifluoromethyl)pyridin-2-yl)-1,2-dihydro-3H-pyrazol-3-one, having the structure of

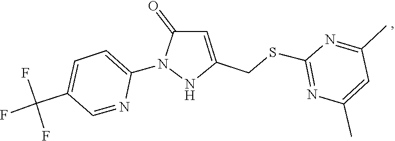 [SMILES: Cc1cc(C)nc(SCc2cc(=O)n(-c3ccc(C(F)(F)F)cn3)[nH]2)n1]

was synthesized. MS (ES⁺) m/z found 382.1 (M+1).

Compound 158: 2-(1H-benzo[d]imidazol-2-yl)-5-(((5-bromopyridin-2-yl)thio)methyl)-1,2-dihydro-3H-pyrazol-3-one, having the structure of

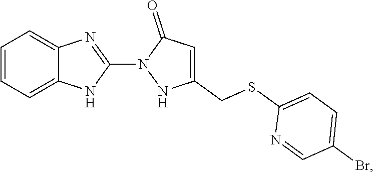 [SMILES: O=c1cc(CSc2ccc(Br)cn2)[nH]n1-c1nc2ccccc2[nH]1]

was synthesized. MS (ES⁺) m/z found 402.0 and 404.0 (M+0; M+2).

Compound 159: 3-(((1-(1H-benzo[d]imidazol-2-yl)-5-oxo-2,5-dihydro-1H-pyrazol-3-yl)methyl)thio)benzamide, having the structure of

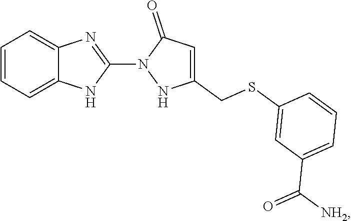 [SMILES: NC(=O)c1cccc(SCc2cc(=O)n(-c3nc4ccccc4[nH]3)[nH]2)c1]

was synthesized. MS (ES⁺) m/z found 366.2 (M+1).

Compound 160: 5-(((5-bromopyridin-2-yl)thio)methyl)-2-(pyridin-2-yl)-1,2-dihydro-3H-pyrazol-3-one, having the structure of

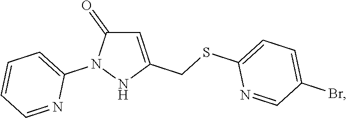 [SMILES: O=c1cc(CSc2ccc(Br)cn2)[nH]n1-c1ccccn1]

was synthesized. MS (ES⁺) m/z found 363.0 and 365.0 (M+0; M+2).

Compound 161: 5-(((4-fluorobenzyl)thio)methyl)-2-(pyridin-2-yl)-1,2-dihydro-3H-pyrazol-3-one, having the structure of

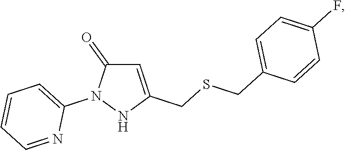 [SMILES: O=c1cc(CSCc2ccc(F)cc2)[nH]n1-c1ccccn1]

was synthesized. MS (ES⁺) m/z found 316.1 (M+1).

Compound 162: 5-(((4-(azidomethyl)phenyl)thio)methyl)-2-(1H-benzo[d]imidazol-2-yl)-1,2-dihydro-3H-pyrazol-3-one, having the structure of

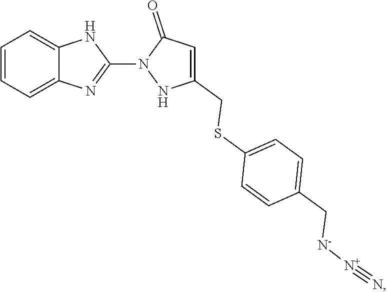 [SMILES: N#[N+][N-]Cc1ccc(SCc2cc(=O)n(-c3nc4ccccc4[nH]3)[nH]2)cc1]

was synthesized. MS (ES⁺) m/z found 377.1.

Compound 163: 2-(1H-benzo[d]imidazol-2-yl)-5-(((4-((4-methylpiperazin-1-yl)methyl)phenyl)thio)methyl)-1,2-dihydro-3H-pyrazol-3-one, having the structure of

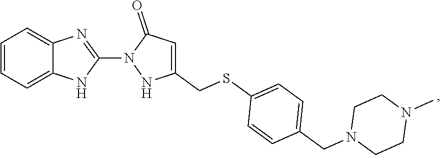 [SMILES: CN1CCN(Cc2ccc(SCc3cc(=O)n(-c4nc5ccccc5[nH]4)[nH]3)cc2)CC1]

was synthesized. MS (ES⁺) m/z found 435.1 (M+1).

Compound 164: 2-(1H-benzo[d]imidazol-2-yl)-5-((((4,6-dimethylpyrimidin-2-yl)methyl)thio)methyl)-1,2-dihydro-3H-pyrazol-3-one, having the structure of

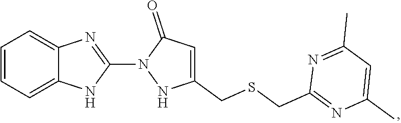 [SMILES: Cc1cc(C)nc(CSCc2cc(=O)n(-c3nc4ccccc4[nH]3)[nH]2)n1]

was synthesized. MS (ES⁺) m/z found 367.1 (M+1).

Compound 165: 5-(((4,6-dimethylpyrimidin-2-yl)thio)methyl)-1-methyl-2-(pyridin-2-yl)-1,2-dihydro-3H-pyrazol-3-one, having the structure of

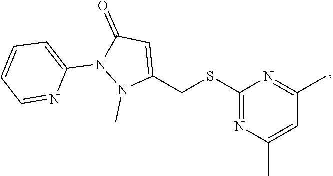 [SMILES: Cc1cc(C)nc(SCc2cc(=O)n(-c3ccccn3)n2C)n1]

was synthesized. MS (ES⁺) m/z found 328.17 (M+1).

Compound 166: 5-((phenylthio)methyl)-2-(pyrazin-2-yl)-1,2-dihydro-3H-pyrazol-3-one, having the structure of

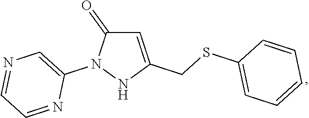 [SMILES: O=c1cc(CSc2ccccc2)[nH]n1-c1cnccn1]

was synthesized. MS (ES⁺) m/z found 284.34 (M+1).

Compound 167: 2-(1H-benzo[d]imidazol-2-yl)-5-(((2-chlorophenyl)thio)methyl)-1,2-dihydro-3H-pyrazol-3-one, having the structure of

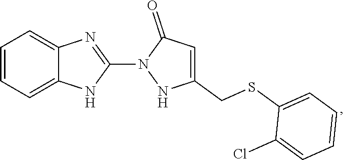 [SMILES: O=c1cc(CSc2ccccc2Cl)[nH]n1-c1nc2ccccc2[nH]1]

was synthesized. MS (ES⁺) m/z found 357.26 (M+1).

Compound 168: 2-(1H-benzo[d]imidazol-2-yl)-5-((pyridin-4-ylthio)methyl)-1,2-dihydro-3H-pyrazol-3-one, having the structure of

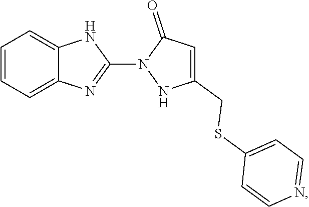 [SMILES: O=c1cc(CSc2ccncc2)[nH]n1-c1nc2ccccc2[nH]1]

was synthesized. MS (ES⁺) m/z found 324.21 (M+1).

Compound 169: 2-(6-methylpyridin-2-yl)-5-((phenylthio)methyl)-1,2-dihydro-3H-pyrazol-3-one, having the structure of

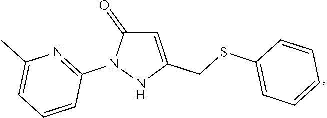 [SMILES: Cc1cccc(-n2[nH]c(CSc3ccccc3)cc2=O)n1]

was synthesized. MS (ES⁺) m/z found 298.1 (M+1).

Compound 170: 2-(1-methyl-1H-benzo[d]imidazol-2-yl)-5-((phenylthio)methyl)-1,2-dihydro-3H-pyrazol-3-one, having the structure of

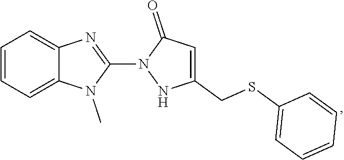 [SMILES: Cn1c(-n2[nH]c(CSc3ccccc3)cc2=O)nc2ccccc21]

was synthesized. MS (ES⁺) m/z found 337.27 (M+1).

Compound 171: 2-(1H-benzo[d]imidazol-2-yl)-5-(((3-chlorophenyl)thio)methyl)-1,2-dihydro-3H-pyrazol-3-one, having the structure of

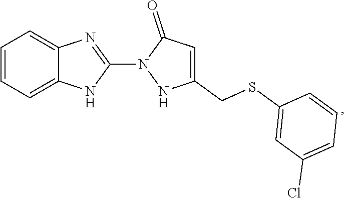 [SMILES: O=c1cc(CSc2cccc(Cl)c2)[nH]n1-c1nc2ccccc2[nH]1]

was synthesized. MS (ES⁺) m/z found 357.31 (M+1).

Compound 172: 2-(1H-benzo[d]imidazol-2-yl)-5-(((3,4-dichlorophenyl)thio)methyl)-1,2-dihydro-3H-pyrazol-3-one, having the structure of

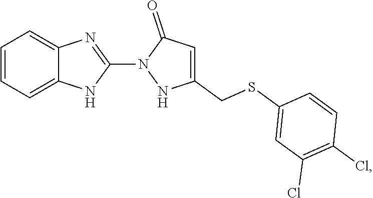 [SMILES: O=c1cc(CSc2ccc(Cl)c(Cl)c2)[nH]n1-c1nc2ccccc2[nH]1]

was synthesized. MS (ES⁺) m/z found 391.18 (M+1).

Compound 173: 3-(((1-(1H-benzo[d]imidazol-2-yl)-5-oxo-2,5-dihydro-1H-pyrazol-3-yl)methyl)thio)benzonitrile, having the structure of

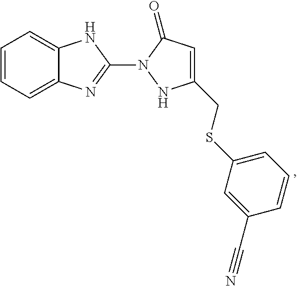 [SMILES: N#Cc1cccc(SCc2cc(=O)n(-c3nc4ccccc4[nH]3)[nH]2)c1]

was synthesized. MS (ES⁺) m/z found 348.24 (M+1).

Compound 174: 2-(4-methylpyridin-2-yl)-5-((phenylthio)methyl)-1,2-dihydro-3H-pyrazol-3-one, having the structure of

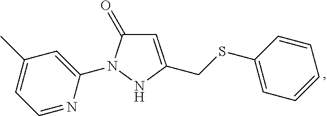 [SMILES: Cc1ccnc(-n2[nH]c(CSc3ccccc3)cc2=O)c1]

was synthesized. MS (ES+) m/z found 298.3 (M+1).

Compound 175: 2-(1H-benzo[d]imidazol-2-yl)-5-(((3,5-dichlorophenyl)thio)methyl)-1,2-dihydro-3H-pyrazol-3-one, having the structure of

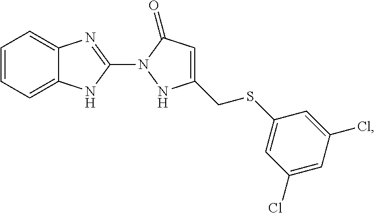 [SMILES: O=c1cc(CSc2cc(Cl)cc(Cl)c2)[nH]n1-c1nc2ccccc2[nH]1]

was synthesized. MS (ES⁺) m/z found 391.1 (M+1).

Compound 176: 2-(4-bromopyridin-2-yl)-5-((phenylthio)methyl)-1,2-dihydro-3H-pyrazol-3-one, having the structure of

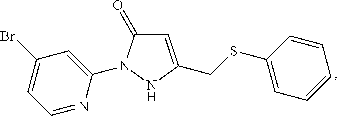 [SMILES: O=c1cc(CSc2ccccc2)[nH]n1-c1cc(Br)ccn1]

was synthesized. MS (ES⁺) m/z found 362.0 and 364.0 (M+0 and M+2).

Compound 177: 2-(1H-benzo[d]imidazol-2-yl)-5-((thiazolo[5,4-b]pyridin-2-ylthio)methyl)-1,2-dihydro-3H-pyrazol-3-one, having the structure of

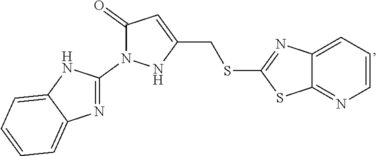 [SMILES: O=c1cc(CSc2nc3cccnc3s2)[nH]n1-c1nc2ccccc2[nH]1]

was synthesized. MS (ES⁺) m/z found 381.1 (M+1).

Compound 178: 4-(((1-(1H-benzo[d]imidazol-2-yl)-5-oxo-2,5-dihydro-1H-pyrazol-3-yl)methyl)thio)benzamide, having the structure of

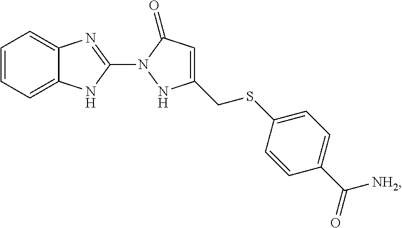 [SMILES: NC(=O)c1ccc(SCc2cc(=O)n(-c3nc4ccccc4[nH]3)[nH]2)cc1]

was synthesized. MS (ES⁺) m/z found 366.2 (M+1).

Compound 179: 2-(1H-imidazol-2-yl)-5-((phenylthio)methyl)-1,2-dihydro-3H-pyrazol-3-one, having the structure of

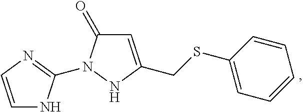 [SMILES: O=c1cc(CSc2ccccc2)[nH]n1-c1ncc[nH]1]

was synthesized. MS (ES⁺) m/z found 273.2 (M+1).

Compound 180: 2-(1H-benzo[d]imidazol-2-yl)-5-(((4-benzylthiazol-2-yl)thio)methyl)-1,2-dihydro-3H-pyrazol-3-one, having the structure of

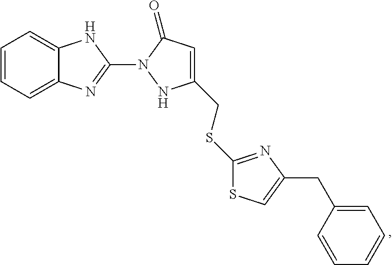 [SMILES: O=c1cc(CSc2nc(Cc3ccccc3)cs2)[nH]n1-c1nc2ccccc2[nH]1]

was synthesized. MS (ES⁺) m/z found 420.2 (M+1).

Compound 181: 2-(1H-benzo[d]imidazol-2-yl)-5-((pyridin-3-ylthio)methyl)-1,2-dihydro-3H-pyrazol-3-one, having the structure of

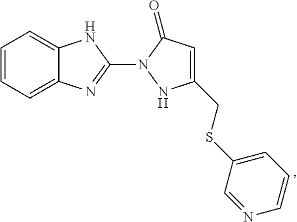 [SMILES: O=c1cc(CSc2cccnc2)[nH]n1-c1nc2ccccc2[nH]1]

was synthesized. MS (ES⁺) m/z found 324.1 (M+1).

Compound 182: 5-(((4-methylthiazol-2-yl)thio)methyl)-2-(pyridin-2-yl)-1,2-dihydro-3H-pyrazol-3-one, having the structure of

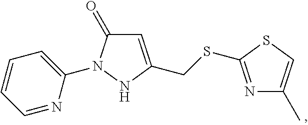 [SMILES: Cc1csc(SCc2cc(=O)n(-c3ccccn3)[nH]2)n1]

was synthesized. MS (ES⁺) m/z found 305.3 (M+1).

Additionally, Compounds 183-187 were prepared. The structures, chemical names, and characterization data for Compounds 183-187 are described below.

Compound 183: 2-(1H-benzo[d]imidazol-2-yl)-5-(((4-chlorophenyl)thio)methyl)-1,2-dihydro-3H-pyrazol-3-one, having the structure of

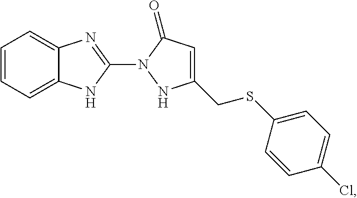 [SMILES: O=c1cc(CSc2ccc(Cl)cc2)[nH]n1-c1nc2ccccc2[nH]1]

was found to have a MS (ES⁺) m/z peak of 358 (M+1).

Compound 184: 2-(benzo[d]thiazol-2-yl)-5-(((4,6-dimethylpyrimidin-2-yl)thio)methyl)-1,2-dihydro-3H-pyrazol-3-one, having the structure of

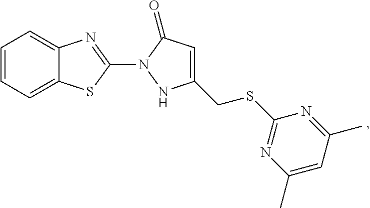 [SMILES: Cc1cc(C)nc(SCc2cc(=O)n(-c3nc4ccccc4s3)[nH]2)n1]

was found to have a MS (ES⁺) m/z peak of 370.2 (M+1).

Compound 185: 2-(benzo[d]thiazol-2-yl)-5-((pyridin-2-ylthio)methyl)-1,2-dihydro-3H-pyrazol-3-one, having the structure of

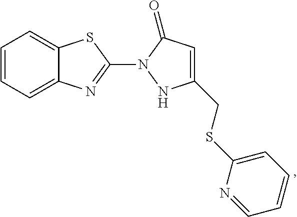 [SMILES: O=c1cc(CSc2ccccn2)[nH]n1-c1nc2ccccc2s1]

was found to have a MS (ES⁺) m/z peak of 341.1 (M+1).

Compound 186: 2-(1H-benzo[d]imidazol-2-yl)-5-((p-tolylthio)methyl)-1,2-dihydro-3H-pyrazol-3-one, having the structure of

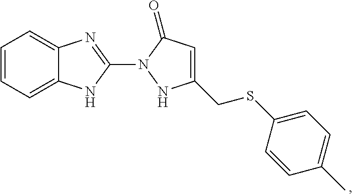 [SMILES: Cc1ccc(SCc2cc(=O)n(-c3nc4ccccc4[nH]3)[nH]2)cc1]

was found to have a MS (ES⁺) m/z peak of 337.1 (M+1).

Compound 187: 2-(benzo[d]thiazol-2-yl)-5-(((5-methyl-1,3,4-thiadiazol-2-yl)thio)methyl)-1,2-dihydro-3H-pyrazol-3-one, having the structure of

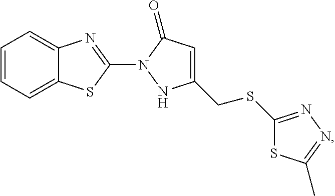 [SMILES: Cc1nnc(SCc2cc(=O)n(-c3nc4ccccc4s3)[nH]2)s1]

was found to have a MS (ES⁺) m/z peak of 362.2 (M+1).

Compound 188: 2-(1H-benzo[d]imidazol-2-yl)-4-methyl-5-(((5-methyl-1,3,4-thiadiazol-2-yl)thio)methyl)-1,2-dihydro-3H-pyrazol-3-one, having the structure of

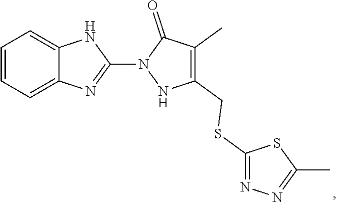 [SMILES: Cc1nnc(SCc2[nH]n(-c3nc4ccccc4[nH]3)c(=O)c2C)s1]

was found to have a MS (ES⁺) m/z peak of 358.4 (M+1).

Compound 189: methyl 2-(4-methyl-3-(((5-methyl-1,3,4-thiadiazol-2-yl)thio)methyl)-5-oxo-2,5-dihydro-1H-pyrazol-1-yl)-1H-benzo[d]imidazole-5-carboxylate, having the structure of

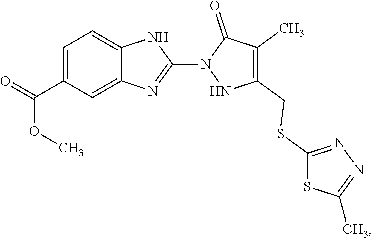 [SMILES: COC(=O)c1ccc2[nH]c(-n3[nH]c(CSc4nnc(C)s4)c(C)c3=O)nc2c1]

was found to have a MS (ES⁺⁾m/z peak of 416.5 (M+1).

### Example 16. Evaluation of Biological Activities: K-Ras Nucleotide Exchange Assay-384 (SOS Exchange Assay by Cytoskeleton (GTPx_Cyt_IC₅₀))

This assay is used to evaluate inhibitors of K-ras, by monitoring the inhibition of nucleotide exchange over time, in the presence of the protein SOS.

### Material

384-well black, untreated plates (Corning 3820 with NBS or 3821 untreated) were used. K-Ras4B Protein was used, which is wild-type (Cytoskeleton #CS-RS03) with a concnetration of 5 mg/ml, or 200 μM. Human SOS1 Protein, Exchange Domain 564-1049 (Cytoskeleton # CS-SOS1-A) was used in a concentration of 3.03 mg/ml or 50 μM. Mant-GDP triethylammonium salt solution was purchased from Sigma (catalog No. 69244). 1M stock solution of Hepes was purchased from Sigma (catalog No. H9897). 1M stock solution of MgCl₂ was purchased from Sigma (catalog No. M4880). CHAPS, 3% was purchased from Sigma (catalog No. C5070). Nonidet P40 was purchased from Sigma. KOH pellets were purchased from Sigma.

K-ras buffer containing 40 mM Hepes, 10 mM MgCl₂, 0.05% CHAPS, 0.01% NP40 with a pH 7.5 was prepared as follows: to a Buffer base containing 4 ml 1M Hepes, 1 ml MgCl₂,

93.3 ml d-H₂O, pH 7.5 (with KOH pellets), 1.7 ml 3% CHAPS was added, followed by the addition of 1 ml 1% NP40.

### Method

Final Conditions of the method included 1 μM KRAS4B (wild type), 0.05 μM SOS1, 1.5 μM mant-GDP. Reagent Preparation included: thawing aliquots of KRAS, SOS, and mant-GDP; preparing 3x KRAS (3 uM) in assay buffer; preparing 3x SOS/mant-GDP (150 nM/4.5 uM) in assay buffer; and preparing 3x compounds in assay buffer.

Assay Procedure (1536) used 3.3 μl, 3x Kras, 3.3 μl 3x compound with preincubate 0 hour, and 3 μl 3x SOS/mant-GDP. A kinetic read measuring fluorescence at 360_450 was performed for 30 min at 30 second intervals.

Reference compound SCH 54292 (reversible inhibitor) at about 100 μM was used with no preincubation. Reagents were only stable for 45 min on ice, and therefore were prepared fresh before each assay. Partial plates (up to 9 columns) were run at a time to minimize effects due to the amount of time it takes to read a whole plate. SOS/mant-GDP were added with an automated pipettor to initiate all reactions at the same time. Assay was typically performed at 0.5% solution in DMSO (compounds prepared in 1.5% DMSO initially).

Clariostar Settings were as follows. Test Name was Kras Kinetic 360-450. Basic settings of the test included Measurement type as Fluorescence (FI), and Microplate name as Corning 3820. Plate mode settings included 61 cycles, 30 second cycle time, and 10 flashes per well. Optic settings were as follows. Excitation wavelength was 360 nm. Emission wavelength was 450 nM. Excitation bandwidth was 12 nm. Emission bandwidth was 12 nm. Dichroic filter 405 was used. D6 Well(s) was used for gain adjustment. No shaking was used. General settings were as follows. Top optic was used. Settling time was 0.1 second. Reading direction was bidirectional, horizontal left to right, and bottom to top. The target temperature was 25° C.

### Example 17. Evaluation of Biological Activities: GDPx_Icagen_ICso

The IC₅₀ assay measures the reduction effect of a given compound on the guanine nucleotide-exchange rate of Ras protein in the presence of guanine nucleotide exchange protein SOS1, which is a Ras activator. In the assay GDP-loaded Ras is first prepared in a buffer containing the compound at certain concentration and fluorescent guanine nucleotide (mant-GDP). The guanine nucleotide exchange was initiated by the addition of the SOS1 and its rate was reflected in the rate of fluorescence change as a function of time, which was monitored by a reader. The experiment was repeated at different compound concentrations and the IC₅₀ of the compound was defined as the compound concentration at which half of its maximal reduction effect on the guanine nucleotide exchange rate is achieved.

Table 5 provides a summary of the IC₅₀ (μM) values of certain exemplified compounds of the instant invention.

TABLE 5
IC₅₀ (μM) values of certain exemplified compounds of the instant invention.

| Compound | | GDPx_Icagen_IC₅₀ | GTPx_Cyt_IC₅₀ |
|---|---|---|---|
| No. | Structure | (μM) | (μM) |
| Compound 139 | 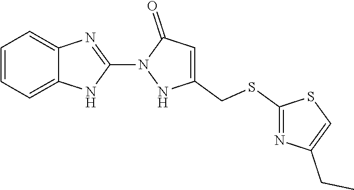 [SMILES: CCc1csc(SCc2cc(=O)n(-c3nc4ccccc4[nH]3)[nH]2)n1] | 0.050 | NT* |
| Compound 140 | 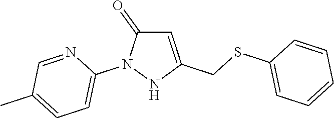 [SMILES: Cc1ccc(-n2[nH]c(CSc3ccccc3)cc2=O)nc1] | 1.6 | 30.8 |
| Compound 141 | 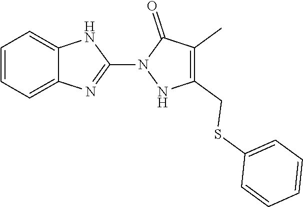 [SMILES: Cc1c(CSc2ccccc2)[nH]n(-c2nc3ccccc3[nH]2)c1=O] | 0.77 | 100 |
| Compound 142 | 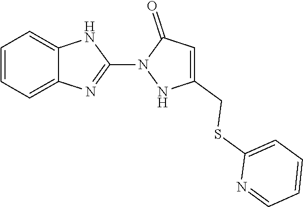 [SMILES: O=c1cc(CSc2ccccn2)[nH]n1-c1nc2ccccc2[nH]1] | 0.17 | 1.2 |
| Compound 143 | 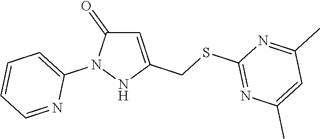 [SMILES: Cc1cc(C)nc(SCc2cc(=O)n(-c3ccccn3)[nH]2)n1] | 2.1 | 5.4 |
| Compound 144 | 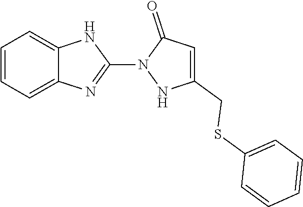 [SMILES: O=c1cc(CSc2ccccc2)[nH]n1-c1nc2ccccc2[nH]1] | 0.30 | 0.42 |
| Compound145 | 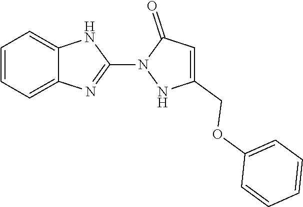 [SMILES: O=c1cc(COc2ccccc2)[nH]n1-c1nc2ccccc2[nH]1] | 0.80 | 3.9 |
| Compound 146 | 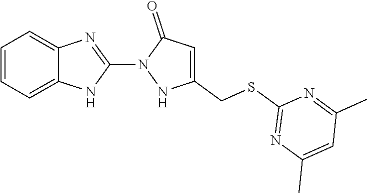 [SMILES: Cc1cc(C)nc(SCc2cc(=O)n(-c3nc4ccccc4[nH]3)[nH]2)n1] | 1.8 | 2.6 |
| Compound 147 | 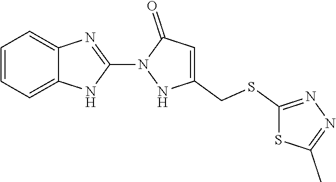 [SMILES: Cc1nnc(SCc2cc(=O)n(-c3nc4ccccc4[nH]3)[nH]2)s1] | 3.3 | 0.85 |
| Compound 148 | 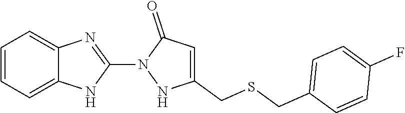 [SMILES: O=c1cc(CSCc2ccc(F)cc2)[nH]n1-c1nc2ccccc2[nH]1] | 0.30 | 0.55 |
| Compound 149 | 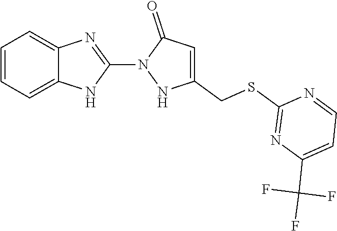 [SMILES: O=c1cc(CSc2nccc(C(F)(F)F)n2)[nH]n1-c1nc2ccccc2[nH]1] | 0.30 | 0.41 |
| Compound 150 | 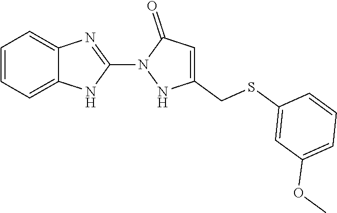 [SMILES: COc1cccc(SCc2cc(=O)n(-c3nc4ccccc4[nH]3)[nH]2)c1] | 0.20 | 0.98 |
| Compound 151 | 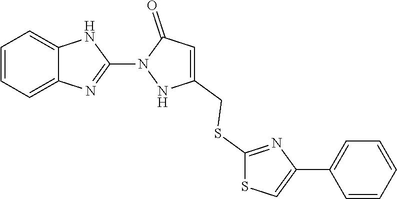 [SMILES: O=c1cc(CSc2nc(-c3ccccc3)cs2)[nH]n1-c1nc2ccccc2[nH]1] | 0.4 | 0.4 |
| Compound 152 | 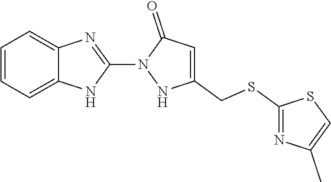 [SMILES: Cc1csc(SCc2cc(=O)n(-c3nc4ccccc4[nH]3)[nH]2)n1] | 0.60 | 0.61 |
| Compound 153 | 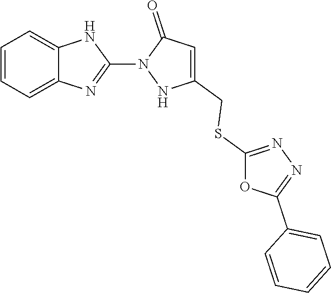 [SMILES: O=c1cc(CSc2nnc(-c3ccccc3)o2)[nH]n1-c1nc2ccccc2[nH]1] | 2.0 | 0.80 |
| Compound 154 | 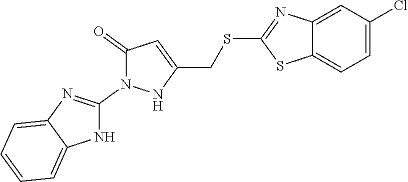 [SMILES: O=c1cc(CSc2nc3cc(Cl)ccc3s2)[nH]n1-c1nc2ccccc2[nH]1] | 0.30 | 1.3 |
| Compound 155 | 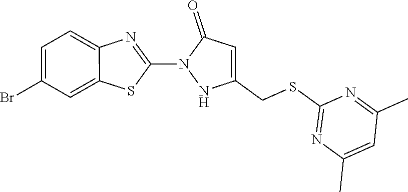 [SMILES: Cc1cc(C)nc(SCc2cc(=O)n(-c3nc4ccc(Br)cc4s3)[nH]2)n1] | NT* | 12.8 |
| Compound 156 | 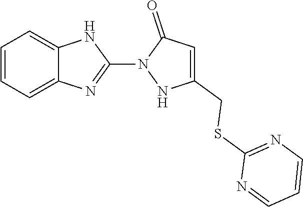 [SMILES: O=c1cc(CSc2ncccn2)[nH]n1-c1nc2ccccc2[nH]1] | 3.0 | 7.0 |
| Compound 157 | 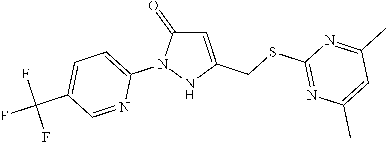 [SMILES: Cc1cc(C)nc(SCc2cc(=O)n(-c3ccc(C(F)(F)F)cn3)[nH]2)n1] | NT* | 17.6 |
| Compound 158 | 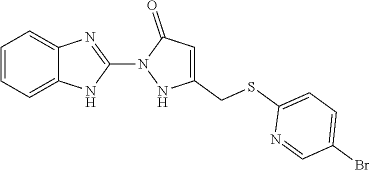 [SMILES: O=c1cc(CSc2ccc(Br)cn2)[nH]n1-c1nc2ccccc2[nH]1] | 0.20 | 0.53 |
| Compound 159 | 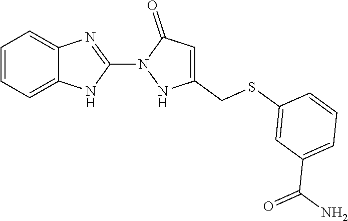 [SMILES: NC(=O)c1cccc(SCc2cc(=O)n(-c3nc4ccccc4[nH]3)[nH]2)c1] | 0.50 | 1.0 |
| Compound 160 | 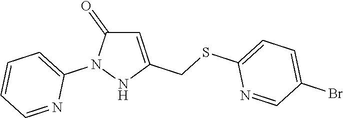 [SMILES: O=c1cc(CSc2ccc(Br)cn2)[nH]n1-c1ccccn1] | NT* | 7.4 |
| Compound 161 | 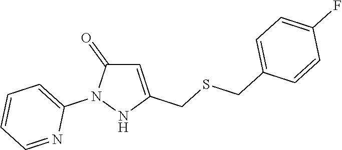 [SMILES: O=c1cc(CSCc2ccc(F)cc2)[nH]n1-c1ccccn1] | NT* | 2.7 |
| Compound 162 | 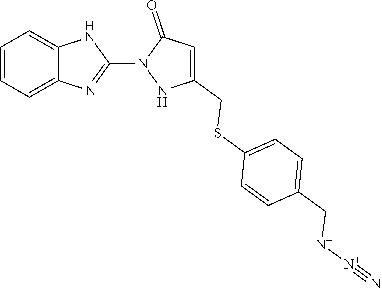 [SMILES: N#[N+][N-]Cc1ccc(SCc2cc(=O)n(-c3nc4ccccc4[nH]3)[nH]2)cc1] | 100 | 1.4 |
| Compound 163 | 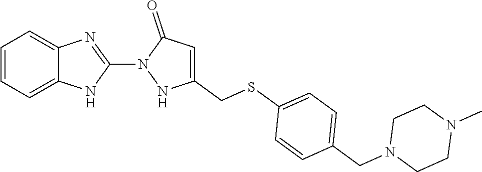 [SMILES: CN1CCN(Cc2ccc(SCc3cc(=O)n(-c4nc5ccccc5[nH]4)[nH]3)cc2)CC1] | 0.50 | 1.0 |
| Compound 164 | 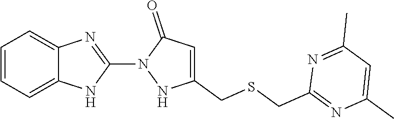 [SMILES: Cc1cc(C)nc(CSCc2cc(=O)n(-c3nc4ccccc4[nH]3)[nH]2)n1] | 22.0 | 0.63 |
| Compound 165 | 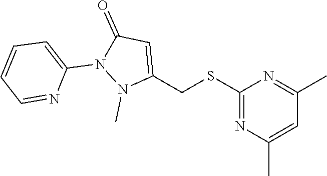 [SMILES: Cc1cc(C)nc(SCc2cc(=O)n(-c3ccccn3)n2C)n1] | 200 | 300 |
| Compound 166 | 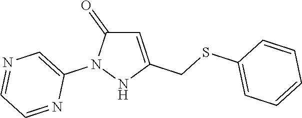 [SMILES: O=c1cc(CSc2ccccc2)[nH]n1-c1cnccn1] | 48.8 | 331 |
| Compound 167 | 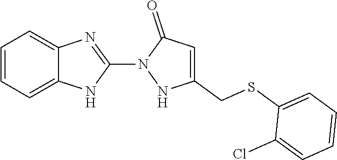 [SMILES: O=c1cc(CSc2ccccc2Cl)[nH]n1-c1nc2ccccc2[nH]1] | 0.200 | 300 |
| Compound 168 | 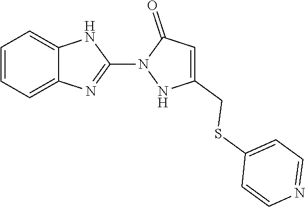 [SMILES: O=c1cc(CSc2ccncc2)[nH]n1-c1nc2ccccc2[nH]1] | 2.3 | 7.0 |
| Compound 169 | 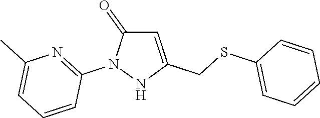 [SMILES: Cc1cccc(-n2[nH]c(CSc3ccccc3)cc2=O)n1] | 7.0 | 2.6 |
| Compound 170 | 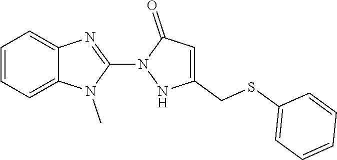 [SMILES: Cn1c(-n2[nH]c(CSc3ccccc3)cc2=O)nc2ccccc21] | 11.9 | 6.1 |
| Compound 171 | 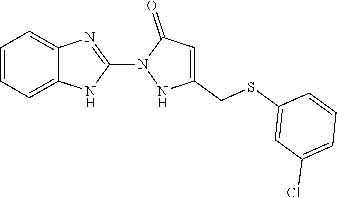 [SMILES: O=c1cc(CSc2cccc(Cl)c2)[nH]n1-c1nc2ccccc2[nH]1] | 0.17 | 100 |
| Compound 172 | 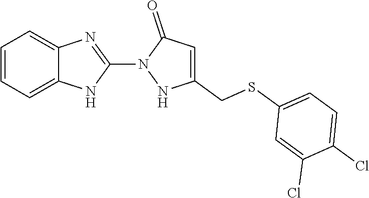 [SMILES: O=c1cc(CSc2ccc(Cl)c(Cl)c2)[nH]n1-c1nc2ccccc2[nH]1] | 0.20 | 0.80 |
| Compound 173 | 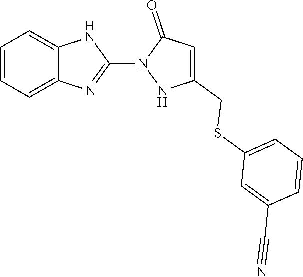 [SMILES: N#Cc1cccc(SCc2cc(=O)n(-c3nc4ccccc4[nH]3)[nH]2)c1] | 0.50 | 100 |
| Compound 174 | 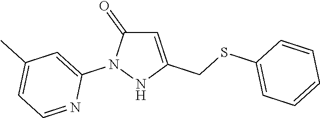 [SMILES: Cc1ccnc(-n2[nH]c(CSc3ccccc3)cc2=O)c1] | 9.3 | 2.7 |
| Compound 175 | 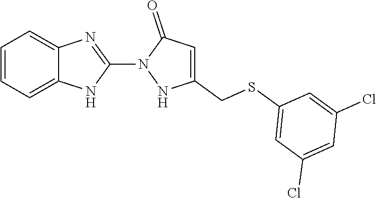 [SMILES: O=c1cc(CSc2cc(Cl)cc(Cl)c2)[nH]n1-c1nc2ccccc2[nH]1] | 0.063 | NT* |
| Compound 176 | 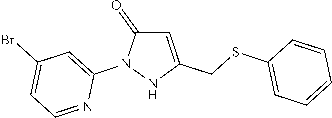 [SMILES: O=c1cc(CSc2ccccc2)[nH]n1-c1cc(Br)ccn1] | 6.0 | NT* |
| Compound 177 | 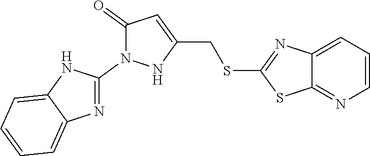 [SMILES: O=c1cc(CSc2nc3cccnc3s2)[nH]n1-c1nc2ccccc2[nH]1] | 0.40 | NT* |
| Compound 178 | 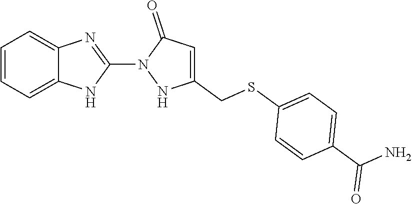 [SMILES: NC(=O)c1ccc(SCc2cc(=O)n(-c3nc4ccccc4[nH]3)[nH]2)cc1] | 0.32 | NT* |
| Compound 179 | 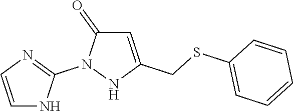 [SMILES: O=c1cc(CSc2ccccc2)[nH]n1-c1ncc[nH]1] | 4.5 | NT* |
| Compound 180 | 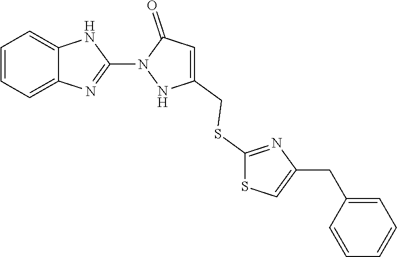 [SMILES: O=c1cc(CSc2nc(Cc3ccccc3)cs2)[nH]n1-c1nc2ccccc2[nH]1] | 0.20 | NT* |
| Compound 181 | 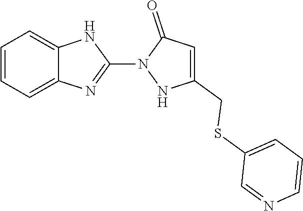 [SMILES: O=c1cc(CSc2cccnc2)[nH]n1-c1nc2ccccc2[nH]1] | 2.5 | NT* |
| Compound 182 | 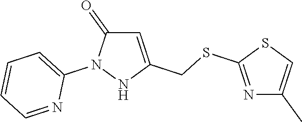 [SMILES: Cc1csc(SCc2cc(=O)n(-c3ccccn3)[nH]2)n1] | 3.2 | NT* |
| Compound 183 | 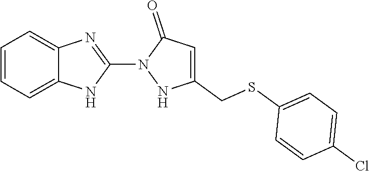 [SMILES: O=c1cc(CSc2ccc(Cl)cc2)[nH]n1-c1nc2ccccc2[nH]1] | 0.20 | 300 |
| Compound 184 | 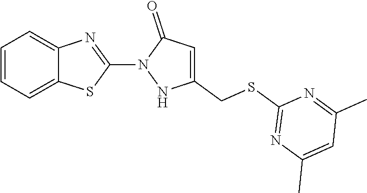 [SMILES: Cc1cc(C)nc(SCc2cc(=O)n(-c3nc4ccccc4s3)[nH]2)n1] | 5.8 | 13.6 |
| Compound 185 | 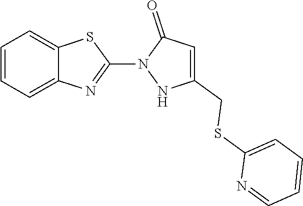 [SMILES: O=c1cc(CSc2ccccn2)[nH]n1-c1nc2ccccc2s1] | 5.9 | 340 |
| Compound 186 | 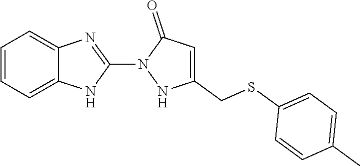 [SMILES: Cc1ccc(SCc2cc(=O)n(-c3nc4ccccc4[nH]3)[nH]2)cc1] | 1.3 | 500 |
| Compound 187 | 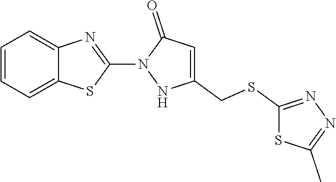 [SMILES: Cc1nnc(SCc2cc(=O)n(-c3nc4ccccc4s3)[nH]2)s1] | 21 | 500 |

*Not Tested.

### Example 18. Evaluation of Biological Activities: Cell ERK Phosphorylation, Thermophoresis, Nucleotide Exchange, and Thermostability

The ERK kinase phosphorylation assay measures the reduction in phosphorylation on ERK kinase for a given compound. In the assay, cells were treated with compounds or DMSO at 37 for 72 hours, washed in PBS buffer once and lysed using SDS loading buffer. The supernatant was then collected by centrifugation, loaded on to a gradient SDS-PAGE, stained against primary anti-pERK antibody and secondary antibody. The level of phosphorylation were qualitatively assessed by the thickness of the bands on gel images.

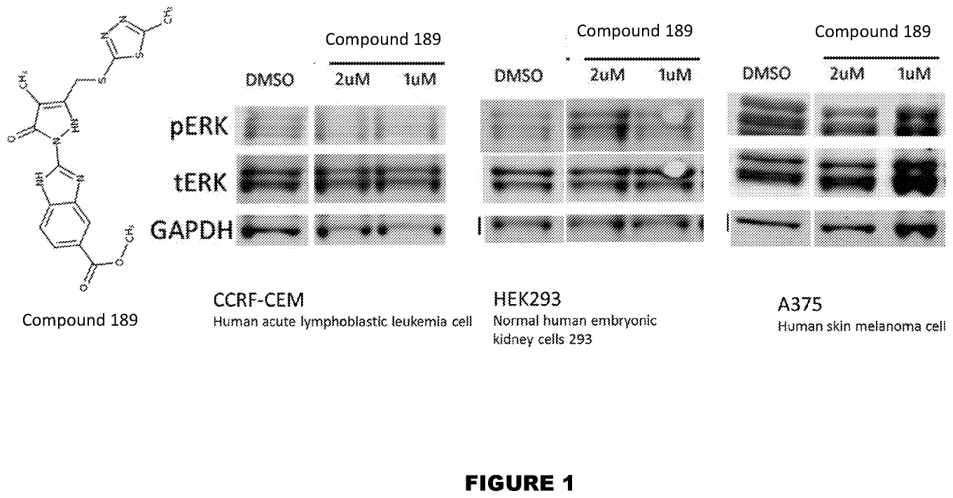

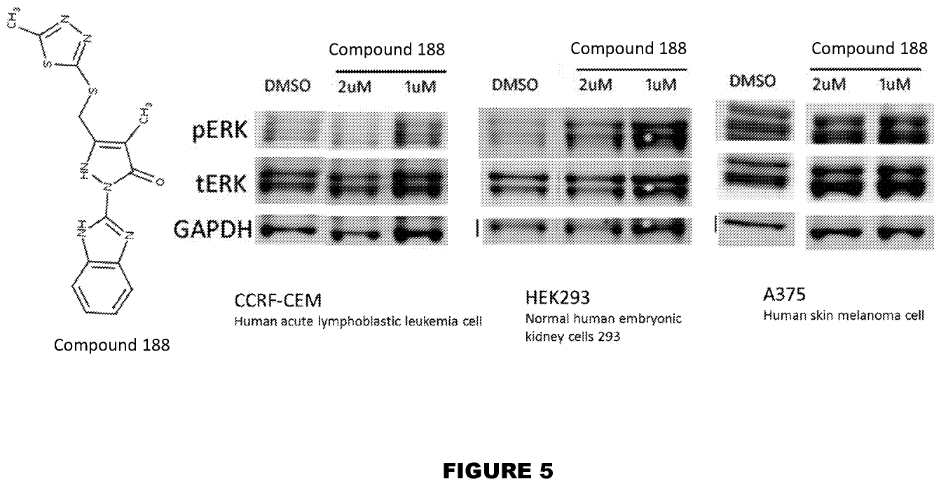

The data in FIG. 1 shows that Compound 189 reduces ERK kinase phosphorylation in non-Raf active cells. The data in FIG. 5 shows that Compound 188 reduces ERK kinase phosphorylation in non-Raf active cells. The CCRF-CEM are human acute lymphoblastic leukemia cells. HEK293 are normal human embryonic kidney cells. A375 are human skin melanoma cells. A375 cell line is a negative control as it is Ras active, and should not be affected by a Ras inhibitor.

The thermophoresis binding assay measures the changes in the movement of KRAS along a temperature gradient as a result of compound binding. In this assay, K-Ras is fluorescently labeled and the thermophoretic movement is measured by monitoring the fluorescence distribution inside a capillary. Compounds of varies concentrations were titrated and the fluorescence distribution signal was measured after 30 seconds or until the diffusion equilibrium is stable. The apparent binding affinity Kd is calculated by fitting the normalized fluorescence distribution values along the concentration axis.

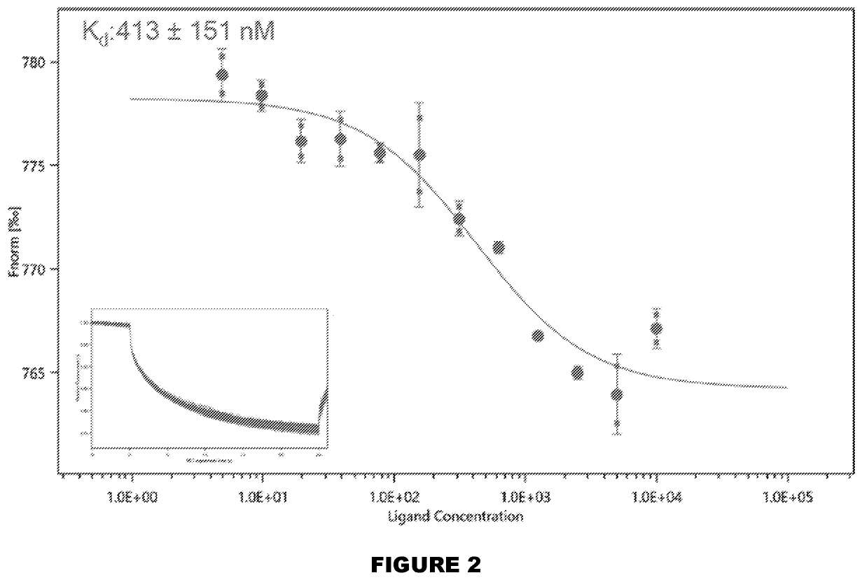

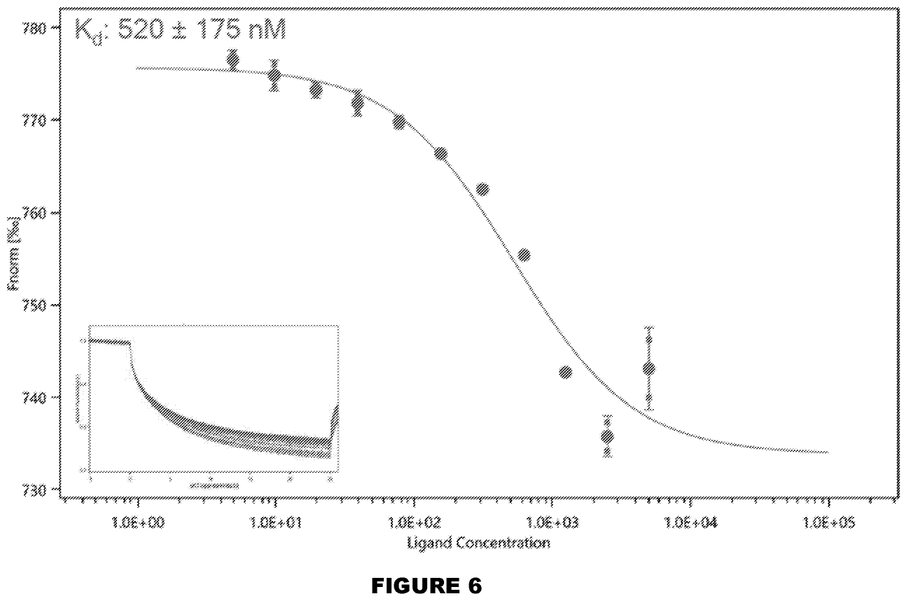

FIG. 2 shows the thermophoresis binding assay for GDP-loaded K-Ras for Compound 189. The K_{d} was 413+151 nM. FIG. 6 shows the thermophoresis binding assay for GDP-loaded K-Ras for Compound 188. The Kd was 520+175 nM.

The K-Ras thermostability assay measures the changes in the thermostability of K-RAS upon compound binding. In this assay, the unfolding curve of K-Ras was generated by measuring the intrinsic fluorescence of tryptophan and tyrosine of the GDP loaded K-Ras using a differential scanning fluorimetry method. The melting temperature Tm was then calculated by calculating the inflection point of the unfolding curve.

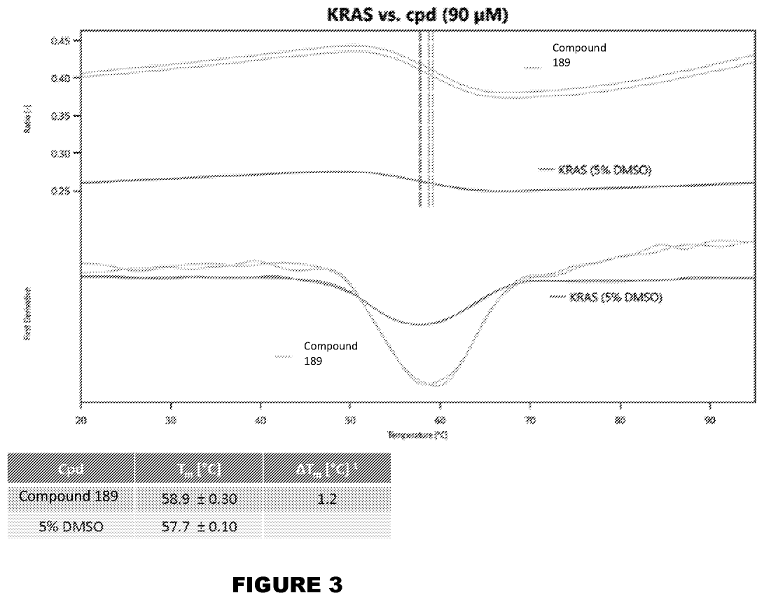

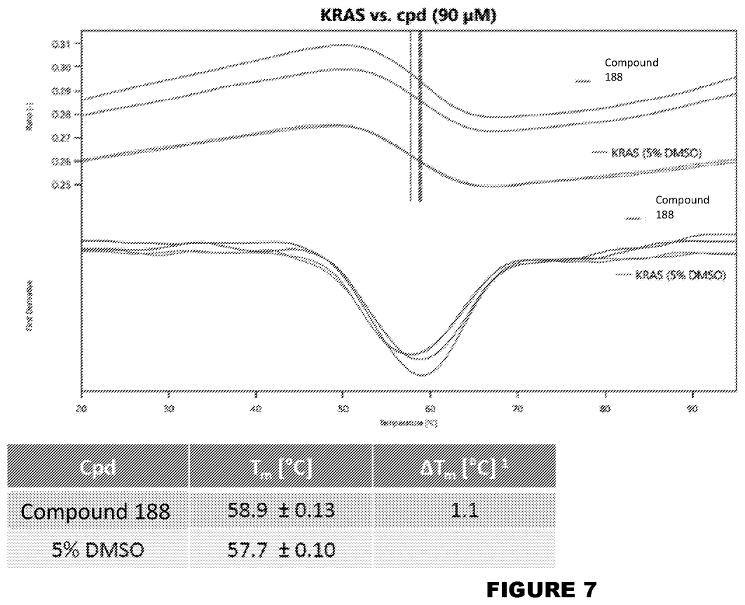

FIG. 3 shows K-Ras thermostability assay was increased by Compound 189. FIG. 7 shows K-Ras thermostability assay was increased by Compound 188.

The IC₅₀ assay measures the reduction effect of a given compound on the guanine nucleotide-exchange rate of Ras protein in the presence of guanine nucleotide exchange protein SOS1, which is a Ras activator. In the assay GDP-loaded Ras is first prepared in a buffer containing the compound at certain concentration and fluorescent guanine nucleotide (mant-GDP). The guanine nucleotide exchange was initiated by the addition of the SOS1 and its rate was reflected in the rate of fluorescence change as a function of time, which was monitored by a reader. The experiment was repeated at different compound concentrations and the IC₅₀ of the compound was defined as the compound concentration at which half of its maximal reduction effect on the guanine nucleotide exchange rate is achieved.

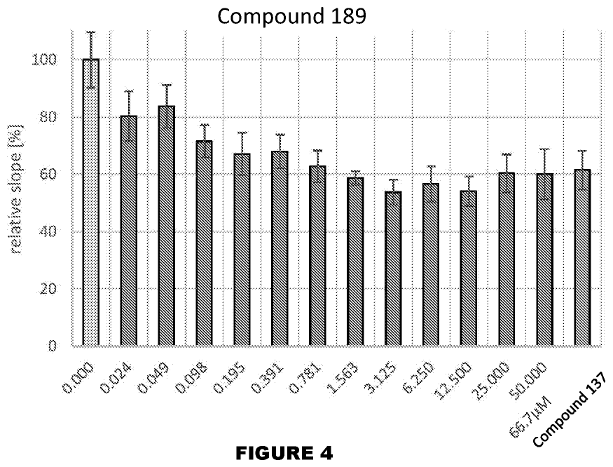

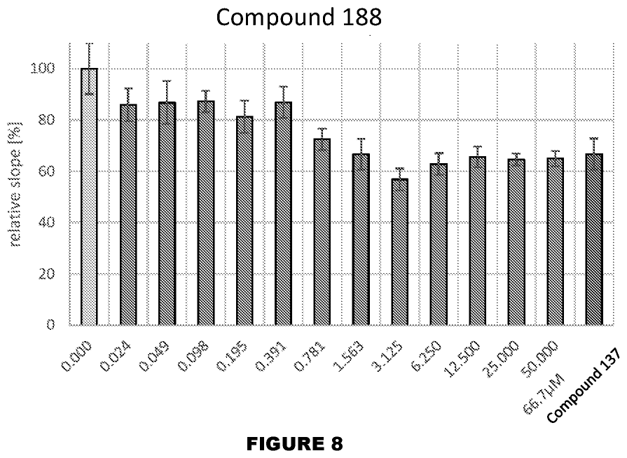

FIG. 4 shows nucleotide exchange rate was reduced by Compound 189. Comparison to nucleotide exchange rate reduction with 66.7 μM of Compound 137 is also shown. Compound 189 has an IC₅₀ of ˜700 nM. FIG. 8 shows nucleotide exchange rate data was reduced by Compound 188. Comparison to nucleotide exchange rate reduction with 66.7 μM of Compound 137 is also shown. Compound 188 has an IC₅₀ of ˜800 nM.

1. A compound of Formula Ia or Ib or a pharmaceutically acceptable salt thereof, 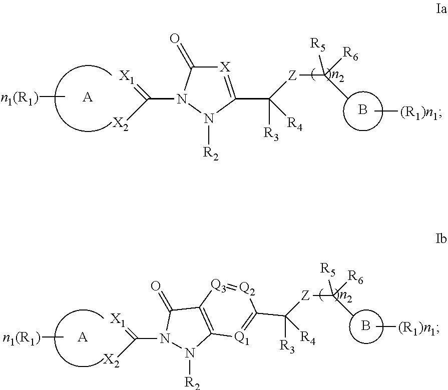
wherein
A is a monocyclic, bicyclic, or tricyclic heterocyclic group optionally substituted by one or more R₁;
each occurrence of n₁ is independently 0, 1, 2, 3, 4, 5, 6, 7, or 8;
X₁ and X₂ are each independently CR₁, O, S, N or NR₂ where valence permits; wherein at least one of X₁ and X₂ is O, N or NR₂;
each occurrence of R₁ is independently hydrogen, halogen, cyano, nitro, —N₃, CF₃, OCF₃, ORₐ, optionally substituted alkyl, optionally substituted cycloalkyl, optionally substituted alkenyl, optionally substituted alkynyl, optionally substituted aryl, optionally substituted heterocycle, —CR_{b}R_{c}-(optionally substituted aryl), —CR_{b}R_{c}-(optionally substituted heteroaryl), —CR_{b}R_{c}—N₃⁻—, SRₐ, S(═O)Rₐ, S(═O)₂Rₐ, —(CR_{b}R_{c})₁₋₄—NR_{b}R_{c}, NR_{b}R_{c}, S(═O)₂NR_{b}R_{c}, oxo, C(═O)ORₐ, C(═O)Rₐ, C(═O)NR_{b}R_{c}, OC(═O)Rₐ, OC(═O)NR_{b}R_{c}, NR_{b}C(═O)ORₐ, or NR_{b}C(═O)Rₐ; or
alternatively two R₁ groups substituted on the same ring taken together form an additional 3-7 membered carbocycle or heterocycle optionally substituted by one or more R_{1′};
each occurrence of R_{1′} is independently hydrogen, halogen, cyano, nitro, CF₃, OCF₃, ORₐ, optionally substituted alkyl, optionally substituted cycloalkyl, optionally substituted alkenyl, optionally substituted aryl, optionally substituted heterocycle, SRₐ, oxo, S(═O)Rₐ, S(═O)₂Rₐ, NR_{b}R_{c}, S(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₐ, or NR_{b}C(═O)Rₐ;
each occurrence of R₂ is independently hydrogen, optionally substituted alkyl, optionally substituted cycloalkyl, optionally substituted alkenyl, optionally substituted cycloalkenyl, optionally substituted alkynyl, optionally substituted heterocycle, or optionally substituted aryl;
X is CR₁ or N;
Q₁, Q₂ and Q₃ are each independently CR₁ or N;
each occurrence of R₃ and R₄ is independently hydrogen, alkyl, cycloalkyl, alkenyl, cycloalkenyl, or alkynyl; or alternatively R₃ and R₄ together with the carbon atom that they are connected to form a 3-7 membered optionally substituted carbocycle or heterocycle;
Z is CR₃R₄, NR₂, O, or S;
n₂ is 0 or 1;
each occurrence of R₅ and R₆ is independently hydrogen, alkyl, cycloalkyl, alkenyl, cycloalkenyl, or alkynyl; or alternatively R₅ and R₆ together with the carbon atom that they are connected to form a 3-7 membered optionally substituted carbocycle or heterocycle;
B is absent, or cycloalkyl group or saturated heterocyclic group optionally substituted by one or more R₁, or monocyclic, bicyclic, or tricyclic aryl or heteroaryl group optionally substituted by one or more R₁; and
Rₐ, R_{b}, and R_{c} are each independently hydrogen, alkyl, cycloalkyl, alkenyl, cycloalkenyl, alkynyl, optionally substituted heterocycle, or optionally substituted aryl; or alternatively R_{b}, and R_{c} together with the nitrogen atom that they are connected to form an optionally substituted heterocycle comprising the nitrogen atom and 0-3 additional heteroatoms each selected from the group consisting of N, O, and S.

2. The compound of claim 1, wherein the compound has the structure of Formula Ia,

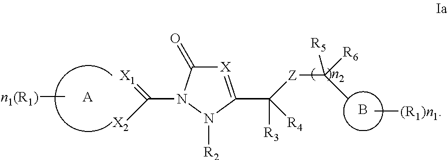

3. The compound of claim 1, wherein the compound has the structure of Formula Ib,

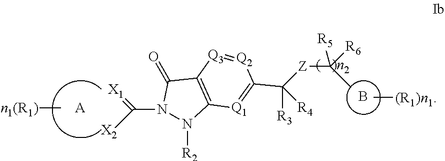

4. The compound of claim 1, wherein X₁ is CR₁ and X₂ is N or NR₂ wherein valence permits.

5. The compound of claim 1, wherein X₁ is N and X₂ is CR₁, NR₂, O or S.

6. The compound of claim 1, wherein X₁ and X₂ are each independently N or NR₂ where valence permits.

7. The compound of claim 1, wherein X₁ is N and X₂ is NR₂.

8. The compound of claim 1, wherein A is an optionally substituted heterocycle selected from the group consisting of pyrrolyl, pyrazolyl, imidazolyl, isoxazolyl, thiazolyl, thiadiazolyl, isothiazolyl, pyridinyl, pyrazinyl, pyrimidinyl, pyridazinyl, benzothiazolyl and benzoimidazolyl.

9. The compound of claim 1, wherein the substituent 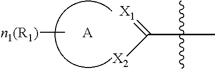
has the structure of

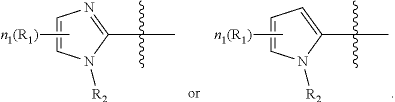

10. The compound of claim 1, wherein the substituent 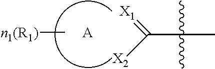
has the structure of

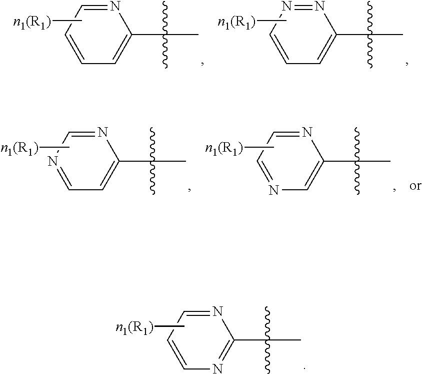

11. The compound of claim 1, wherein the substituent 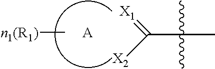
has the structure of

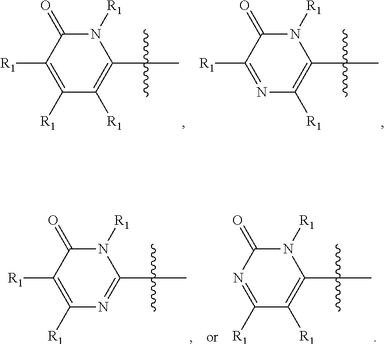

12. The compound of claim 1, wherein the substituent 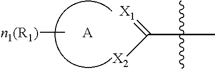
has the structure of

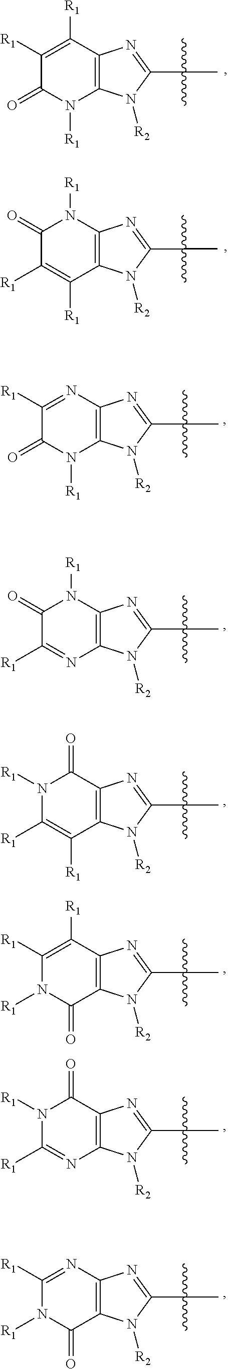

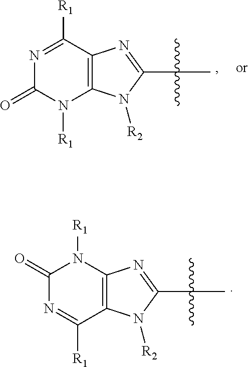

13. The compound of claim 1, wherein the substituent 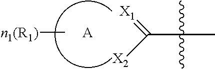
has the structure of

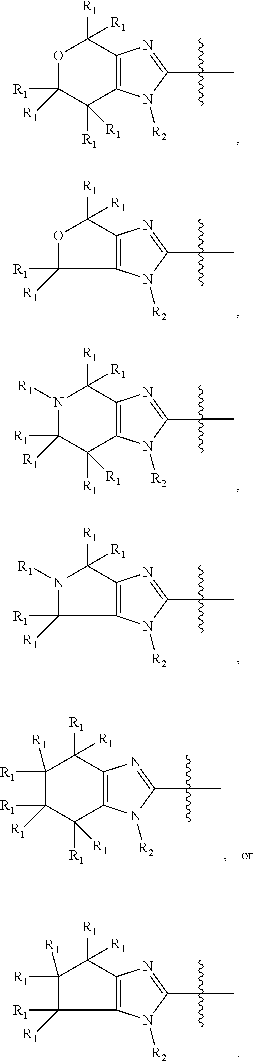

14. The compound of claim 1, wherein the substituent 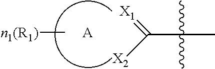
has the structure of 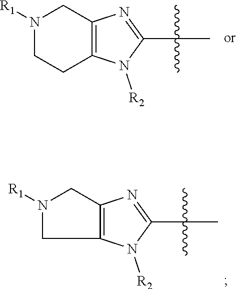
and wherein R₁ is H, NH₂, alkyl, cycloalkyl, aryl, heterocycle, or C(═O)Rₐ.

15. The compound of claim 1, wherein the substituent 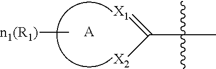
has the structure of

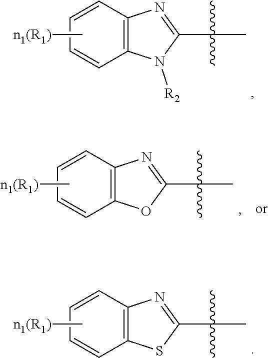

16. The compound of claim 1, wherein the substituent 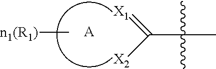
has the structure of

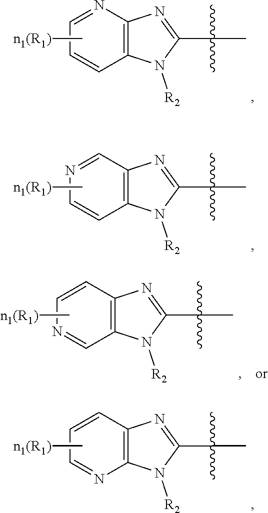

17. The compound of claim 1, wherein the substituent 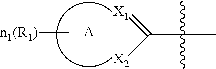
has the structure of

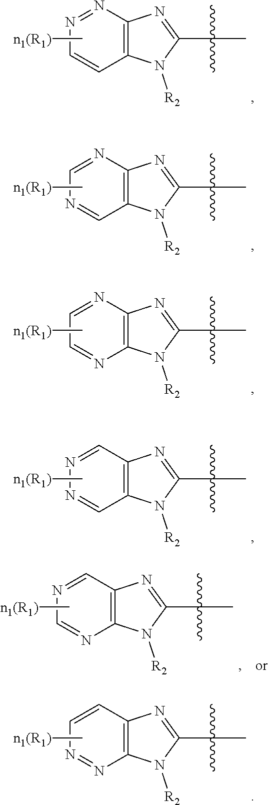

18. The compound of claim 1, wherein X is CR₁.

19. The compound of claim 1, wherein X is N.

20. The compound of claim 1, wherein Q₁, Q₂ and Q₃ are each CR₁.

21. The compound of claim 1, wherein Q₁, Q₂ and Q₃ are each N.

22. The compound of claim 1, wherein Q₁, Q₂ and Q₃ are, respectively, N, CR₁, and CR₁; or Q₁, Q₂ and Q₃ are, respectively, CR₁, N, and CR₁; or Q₁, Q₂ and Q₃ are, respectively, CR₁, CR₁, and N; or Q₁, Q₂ and Q₃ are, respectively, N, N, and CR₁; or Q₁, Q₂ and Q₃ are, respectively, N, CR₁, and N; or Q₁, Q₂ and Q₃ are, respectively, CR₁, N, and N.

23. The compound of claim 1, wherein R₃ and R₄ are each H, methyl, or ethyl.

24. The compound of claim 1, wherein R₃ and R₄ are each H.

25. The compound of claim 1, wherein Z is S.

26. The compound of claim 1, wherein Z is O.

27. The compound of claim 1, wherein Z is CR₃R₄.

28. The compound of claim 27, wherein Z is CH₂, CHMe, or CMe₂.

29. The compound of claim 1, wherein Z is NR₂.

30. The compound of claim 29, wherein Z is NH or NMe.

31. The compound claim 1, wherein n₂ is 0.

32. The compound of claim 1, wherein n₂ is 1.

33. The compound of claim 32, wherein R₅ and R₆ are, respectively, H and H; or R₅ and R₆ are, respectively, H and Me; or R₅ and R₆ are, respectively, Me and Me.

34. The compound of claim 1, wherein B is cycloalkyl, saturated heterocycle, 5-membered heteroaryl, 6-membered aryl, 6-membered heteroaryl, fused bicyclic aryl or heteroaryl, fused tricyclic aryl or heteroaryl, aryl-aryl, heteroaryl-aryl, heteroaryl-heteroaryl, or aryl-heteroaryl, each of which is optionally substituted by one or more R₁.

35. The compound of claim 34, wherein B is optionally substituted cyclohexyl, 4-morpholinyl, N-methylpyperizinyl, thiazolyl, thiadiazolyl, oxyzolyl, pyrrolyl, pyrozolyl, phenyl, pyridyl, phenyl-thiazolyl, phenyl-thiadiazolyl, pyridinyl-thiazolyl, pyridinyl-thiadiazolyl, benzothiazolyl, pyrimidinyl, phenyl-oxadiazolyl, or thiazolopyridinyl.

36. The compound of claim 1, having the structure of Formula IIa or IIb,

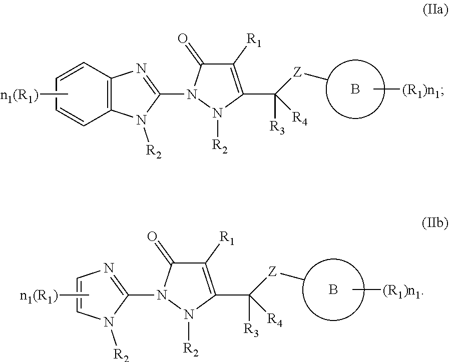

37. The compound of claim 1, having the structure of Formula IIIa or IIIb,

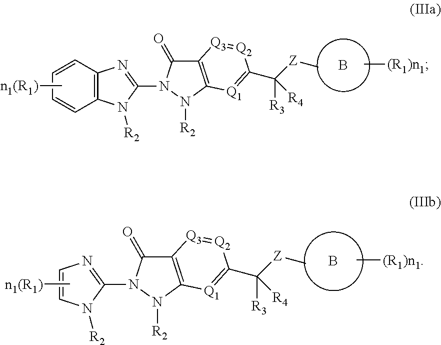

38. The compound of claim 1, wherein one or more occurrences of R₁ are independently H, Me, Et, F, Cl, Br, CF₃, OH, NH₂, Ph, or pyridinyl.

39. The compound of claim 1, wherein one or more occurrences of R₁ are independently H, F, Cl or methyl.

40. The compound of claim 1, wherein R₁ is H.

41. The compound of claim 1, wherein each occurrence of R₂ is independently H, alkyl, aryl or heteroaryl.

42. The compound of claim 1, wherein each occurrence of R₂ is independently H, Me, Ph, or pyridinyl.

43. The compound of claim 1, wherein each occurrence of R₂ is independently H, Me, or Et.

44. The compound of claim 1, wherein R₂ is H.

45. The compound of claim 1, wherein Z is NR₂; n₂ is 0; B is absent; and a R₁ substituent is connected to Z and the R₁ substituent is S(═O)Rₐ, S(═O)₂Rₐ, S(═O)₂NR_{b}R_{c}, C(═O)ORₐ, C(═O)Rₐ, C(═O)NR_{b}R_{c}, OC(═O)Rₐ, or OC(═O)NR_{b}R_{c}.

46. The compound of claim 1, wherein NR_{b}R_{c} is a heterocycle selected from the group consisting of 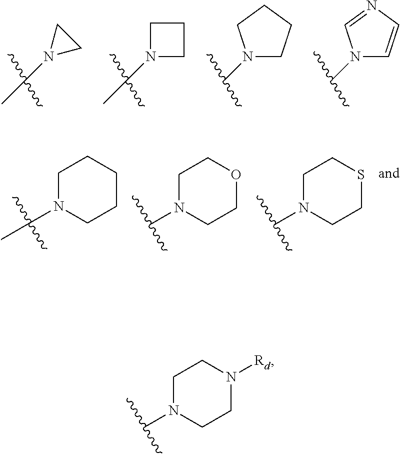
wherein R_{d} is H, Me, Et, n-Pr, i-Pr, n-Bu, i-Bu, t-Bu, CH₂CMe₃, Ph, CH₂Ph, or C(═O)(C₁-C₆ alkyl).

47. The compound of claim 1, wherein NR_{b}R_{c} is NH₂, NHMe, NMe₂, NEt₂, or NH(propyl).

48. The compound of claim 1, wherein 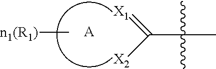
has a structure selected from the group consisting of

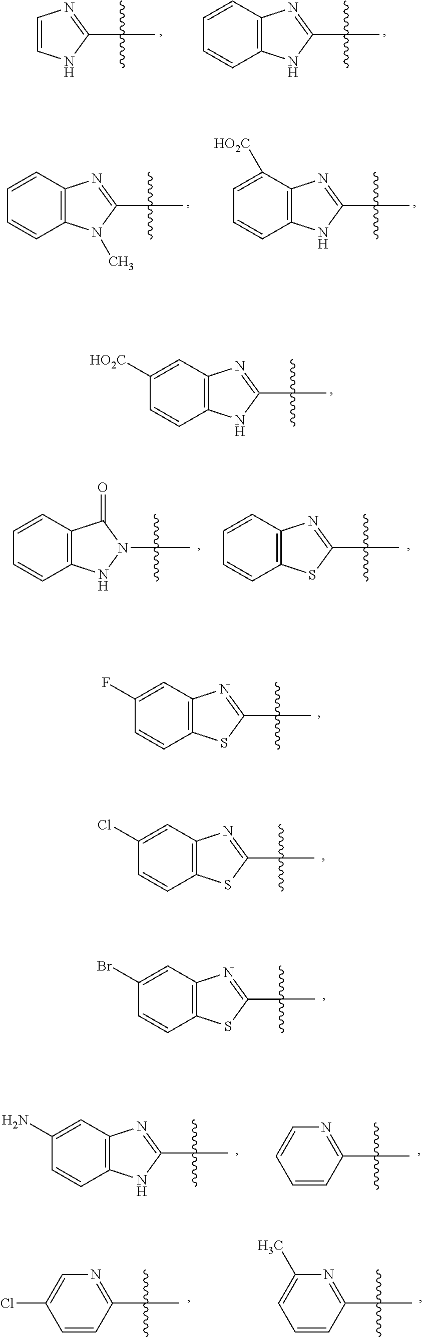

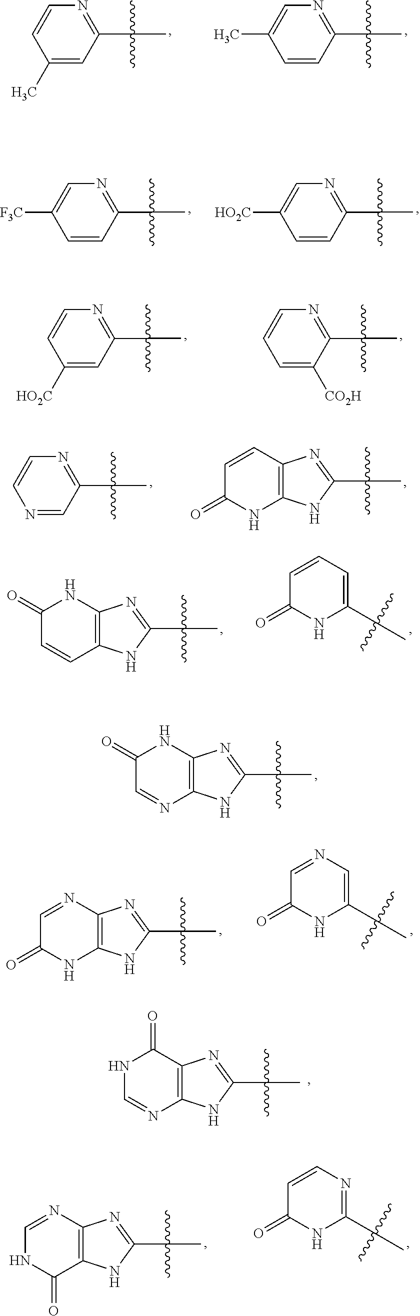

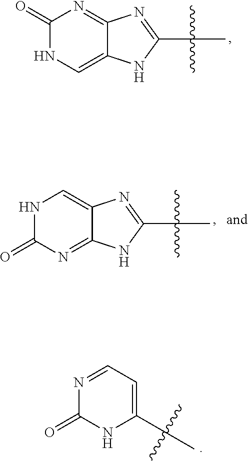 [SMILES: *c1nc2nc(=O)[nH]cc2[nH]1 ; *c1nc2c[nH]c(=O)nc2[nH]1 ; *c1ccnc(=O)[nH]1]

49. The compound of claim 1, wherein 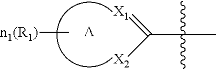
has a structure selected from the group consisting of 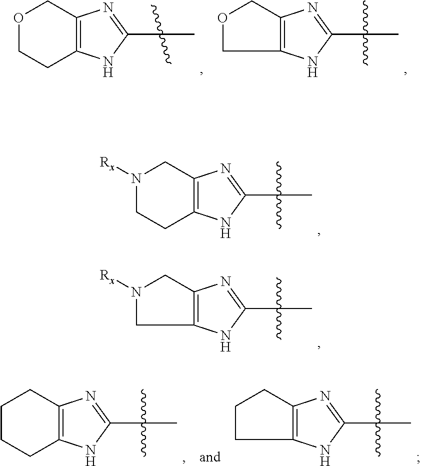
and wherein Rₓ is aryl, heteroaryl, cycloalkyl, saturated heterocycle, or C(═O)Rₐ.

50. The compound of claim 1, wherein B has a structure selected from the group consisting of

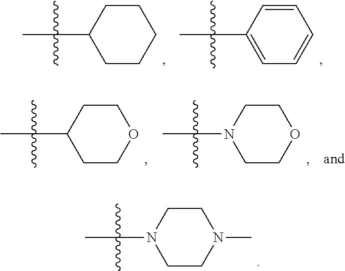

51. The compound of claim 1, wherein B has a structure selected from the group consisting of

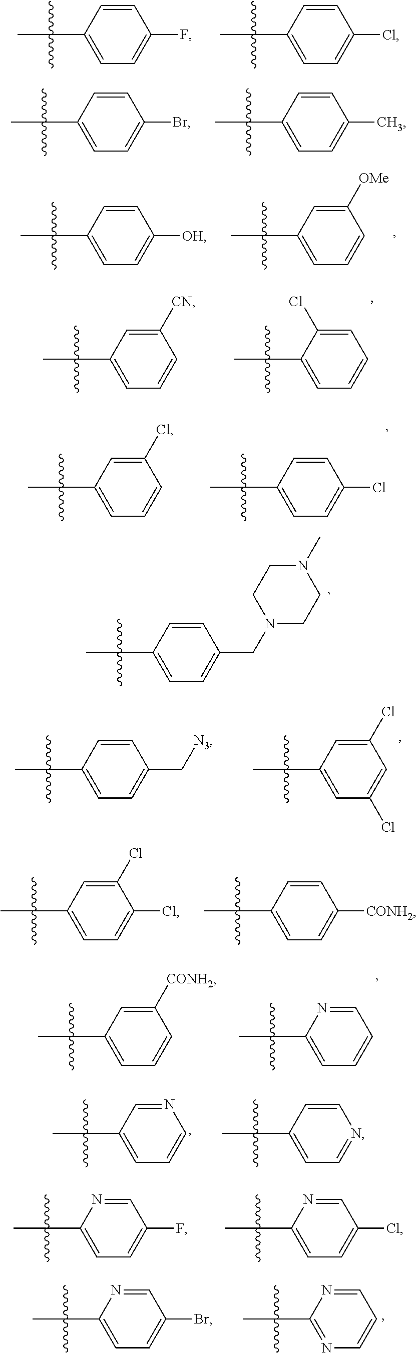

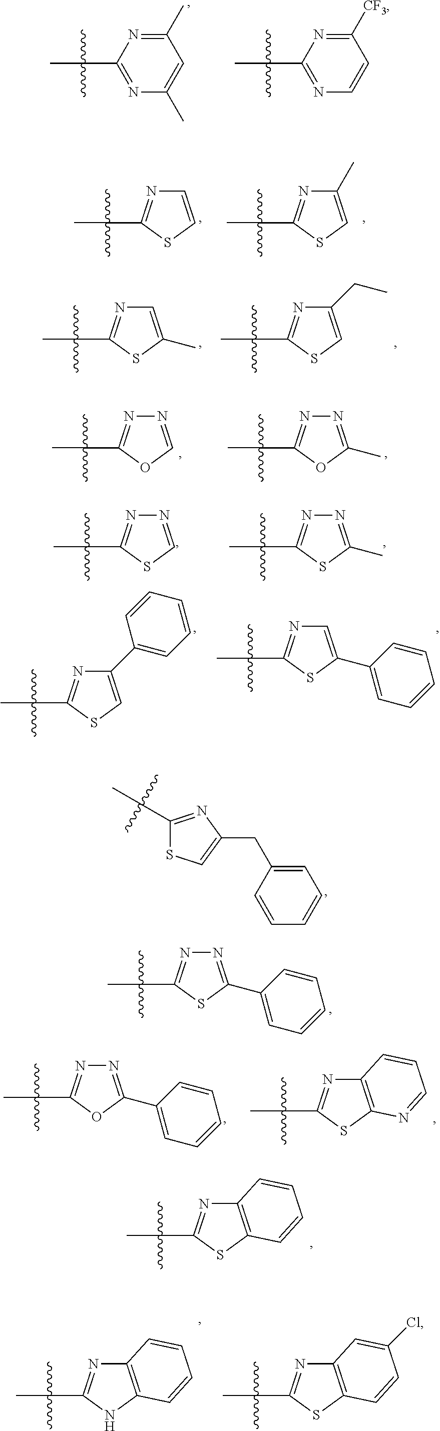

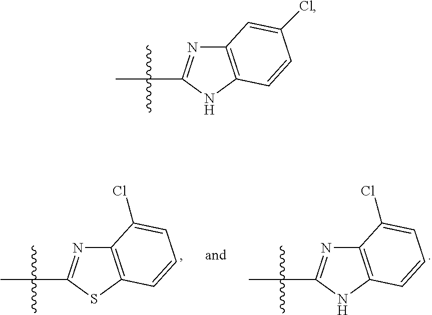 [SMILES: *c1nc2cc(Cl)ccc2[nH]1 ; *c1nc2c(Cl)cccc2s1 ; *c1nc2c(Cl)cccc2[nH]1]

52. The compound of claim 1, wherein substituent 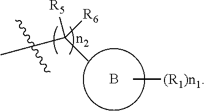
has the structure of

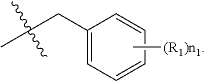

53. The compound of claim 1, wherein the compound is selected from a group consisting of:

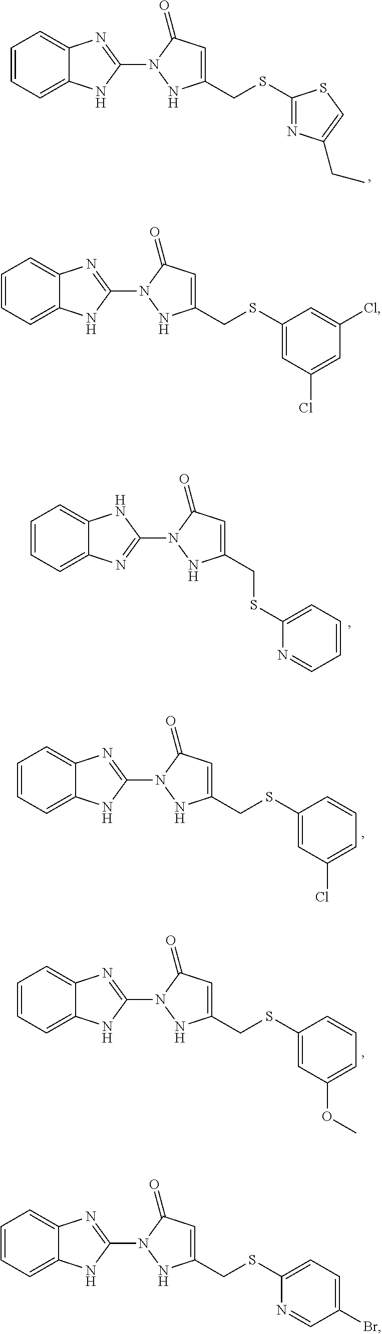 [SMILES: CCc1csc(SCc2cc(=O)n(-c3nc4ccccc4[nH]3)[nH]2)n1 ; O=c1cc(CSc2cc(Cl)cc(Cl)c2)[nH]n1-c1nc2ccccc2[nH]1 ; O=c1cc(CSc2ccccn2)[nH]n1-c1nc2ccccc2[nH]1 ; O=c1cc(CSc2cccc(Cl)c2)[nH]n1-c1nc2ccccc2[nH]1 ; COc1cccc(SCc2cc(=O)n(-c3nc4ccccc4[nH]3)[nH]2)c1 ; O=c1cc(CSc2ccc(Br)cn2)[nH]n1-c1nc2ccccc2[nH]1]

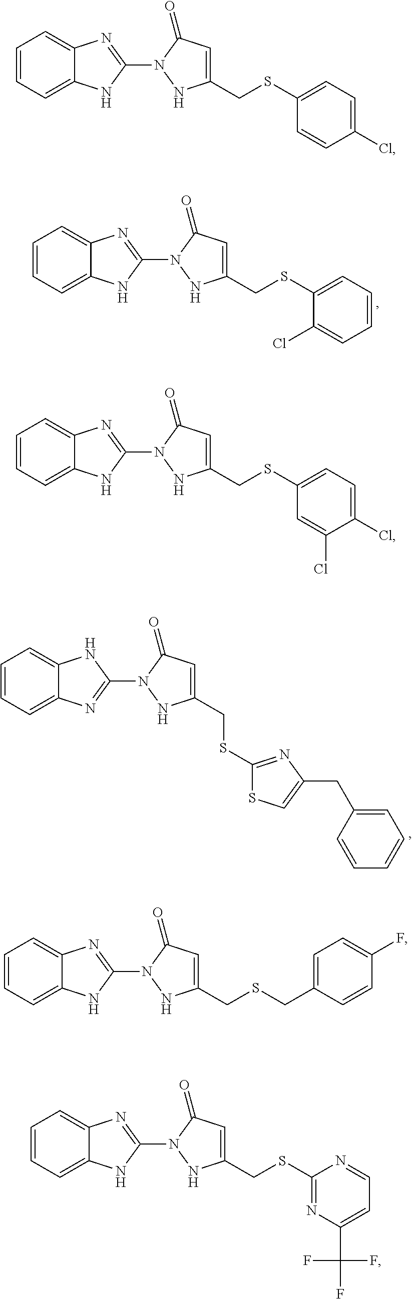 [SMILES: O=c1cc(CSc2ccc(Cl)cc2)[nH]n1-c1nc2ccccc2[nH]1 ; O=c1cc(CSc2ccccc2Cl)[nH]n1-c1nc2ccccc2[nH]1 ; O=c1cc(CSc2ccc(Cl)c(Cl)c2)[nH]n1-c1nc2ccccc2[nH]1 ; O=c1cc(CSc2nc(Cc3ccccc3)cs2)[nH]n1-c1nc2ccccc2[nH]1 ; O=c1cc(CSCc2ccc(F)cc2)[nH]n1-c1nc2ccccc2[nH]1 ; O=c1cc(CSc2nccc(C(F)(F)F)n2)[nH]n1-c1nc2ccccc2[nH]1]

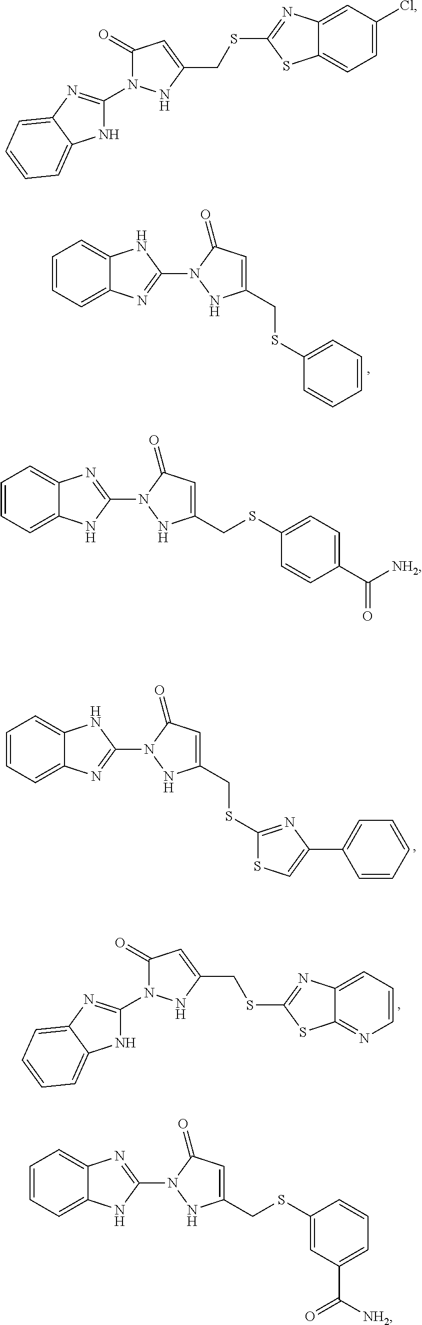 [SMILES: O=c1cc(CSc2nc3cc(Cl)ccc3s2)[nH]n1-c1nc2ccccc2[nH]1 ; O=c1cc(CSc2ccccc2)[nH]n1-c1nc2ccccc2[nH]1 ; NC(=O)c1ccc(SCc2cc(=O)n(-c3nc4ccccc4[nH]3)[nH]2)cc1 ; O=c1cc(CSc2nc(-c3ccccc3)cs2)[nH]n1-c1nc2ccccc2[nH]1 ; O=c1cc(CSc2nc3cccnc3s2)[nH]n1-c1nc2ccccc2[nH]1 ; NC(=O)c1cccc(SCc2cc(=O)n(-c3nc4ccccc4[nH]3)[nH]2)c1]

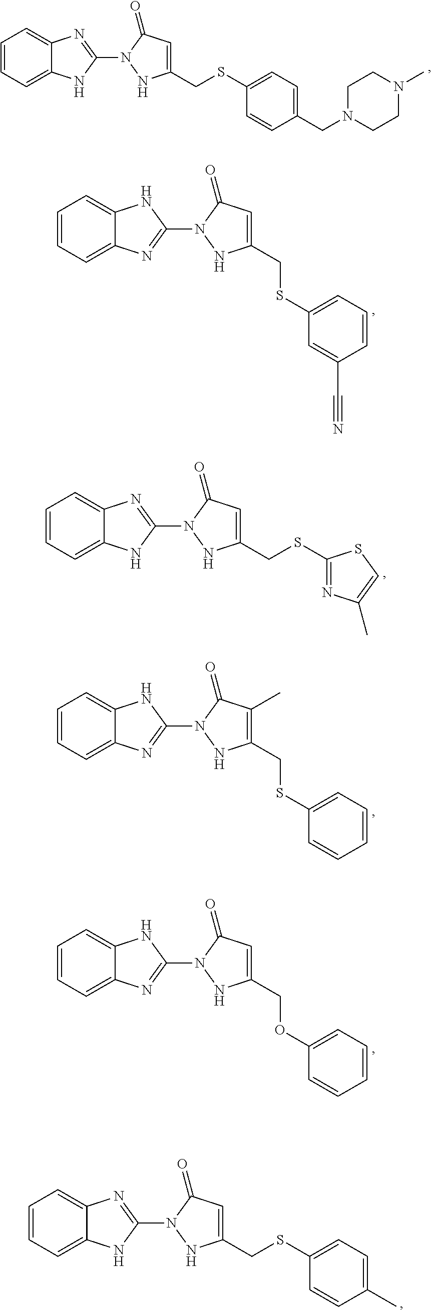 [SMILES: CN1CCN(Cc2ccc(SCc3cc(=O)n(-c4nc5ccccc5[nH]4)[nH]3)cc2)CC1 ; N#Cc1cccc(SCc2cc(=O)n(-c3nc4ccccc4[nH]3)[nH]2)c1 ; Cc1csc(SCc2cc(=O)n(-c3nc4ccccc4[nH]3)[nH]2)n1 ; Cc1c(CSc2ccccc2)[nH]n(-c2nc3ccccc3[nH]2)c1=O ; O=c1cc(COc2ccccc2)[nH]n1-c1nc2ccccc2[nH]1 ; Cc1ccc(SCc2cc(=O)n(-c3nc4ccccc4[nH]3)[nH]2)cc1]

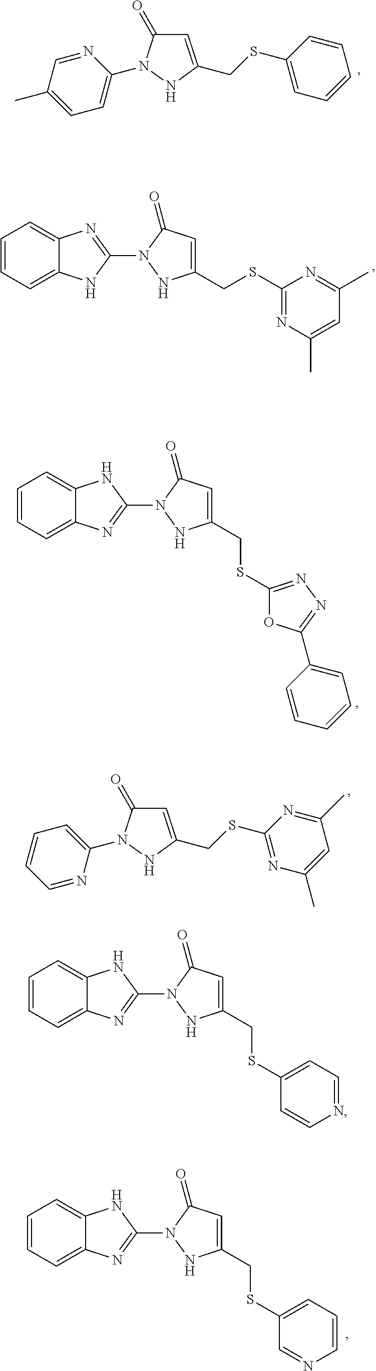 [SMILES: Cc1ccc(-n2[nH]c(CSc3ccccc3)cc2=O)nc1 ; Cc1cc(C)nc(SCc2cc(=O)n(-c3nc4ccccc4[nH]3)[nH]2)n1 ; O=c1cc(CSc2nnc(-c3ccccc3)o2)[nH]n1-c1nc2ccccc2[nH]1 ; Cc1cc(C)nc(SCc2cc(=O)n(-c3ccccn3)[nH]2)n1 ; O=c1cc(CSc2ccncc2)[nH]n1-c1nc2ccccc2[nH]1 ; O=c1cc(CSc2cccnc2)[nH]n1-c1nc2ccccc2[nH]1]

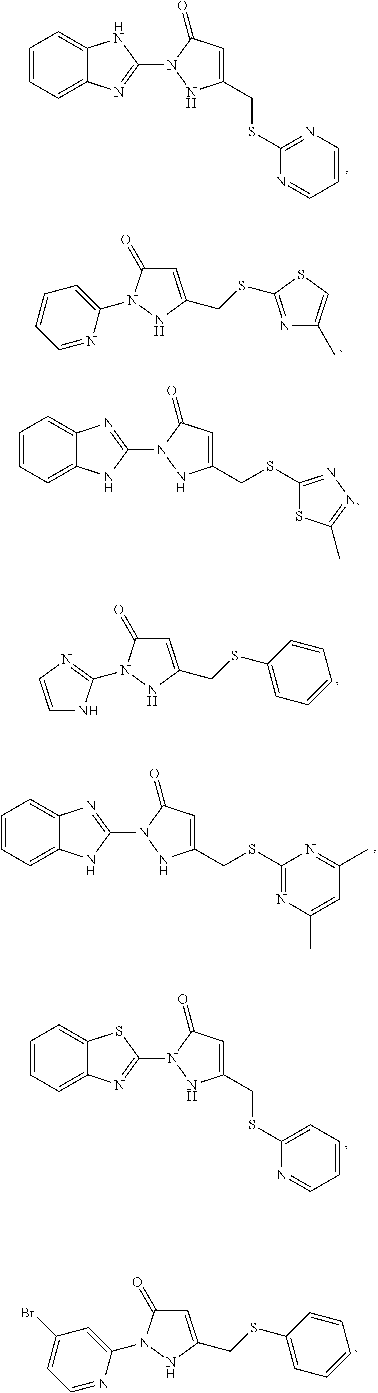

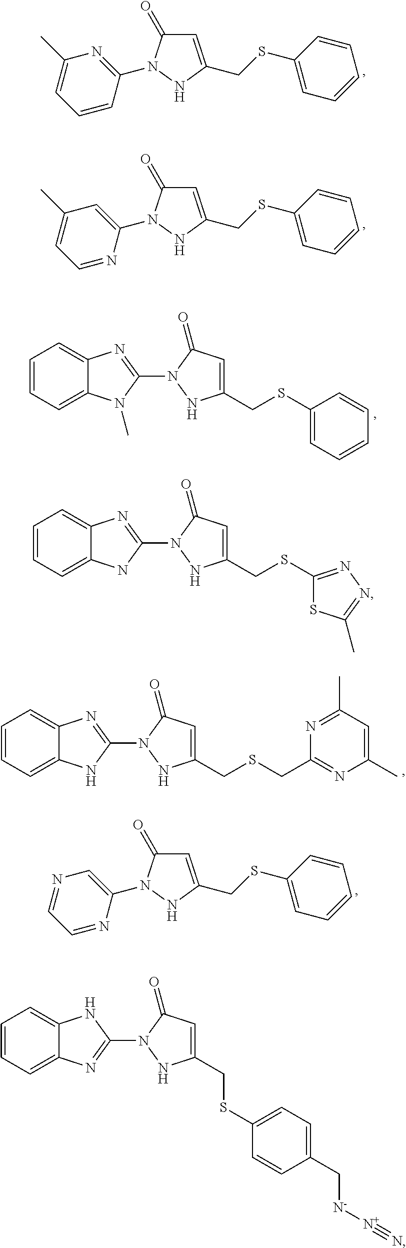

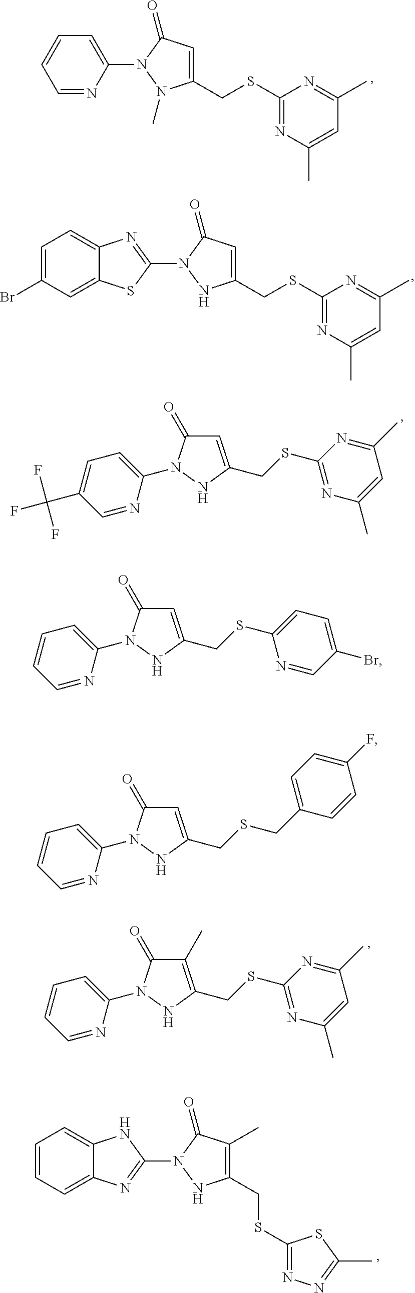

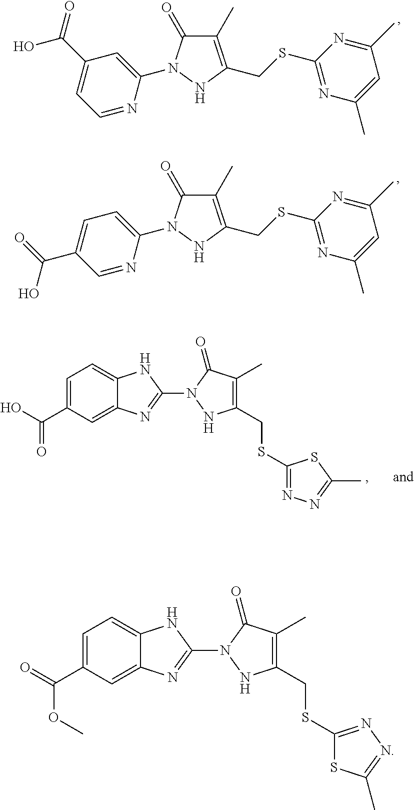 [SMILES: Cc1cc(C)nc(SCc2[nH]n(-c3cc(C(=O)O)ccn3)c(=O)c2C)n1 ; Cc1cc(C)nc(SCc2[nH]n(-c3ccc(C(=O)O)cn3)c(=O)c2C)n1 ; Cc1nnc(SCc2[nH]n(-c3nc4cc(C(=O)O)ccc4[nH]3)c(=O)c2C)s1 ; COC(=O)c1ccc2[nH]c(-n3[nH]c(CSc4nnc(C)s4)c(C)c3=O)nc2c1]

54. The compound of claim 1 selected from the group consisting of Compounds 1-136 in Tables 1-4.

55. A pharmaceutical composition comprising at least one compound according to claim 1 and a pharmaceutically acceptable carrier or diluent.

56. A method of treating cancer in a mammalian species in need thereof, comprising administering to the mammalian species a therapeutically effective amount of at least one compound according to claim 1.

57. The method of claim 56, wherein the mammalian species is human.

58. The method of claim 56, wherein the cancer is selected from the group consisting of biliary tract cancer, brain cancer, breast cancer, cervical cancer, choriocarcinoma, colon cancer, endometrial cancer, esophageal cancer, gastric (stomach) cancer, intraepithelial neoplasms, leukemias, lymphomas, liver cancer, lung cancer, melanoma, neuroblastomas, oral cancer, ovarian cancer, pancreatic cancer, prostate cancer, rectal cancer, renal (kidney) cancer, sarcomas, skin cancer, testicular cancer, and thyroid cancer.

59. A method of inhibiting Ras protein in a mammalian species in need thereof, comprising administering to the mammalian species a therapeutically effective amount of at least one compound according to claim 1.

60. The method of claim 59, wherein the mammalian species is human.
